(19) United States
(12) Patent Application Publication
(10) Pub. No.: US 2024/0357922 A1
(43) Pub. Date: Oct. 24, 2024

(54) LIGHT EMITTING ELEMENT AND HETERO COMPOUND FOR LIGHT EMITTING ELEMENT

(71) Applicant: Samsung Display Co., Ltd., Yongin-si (KR)

(72) Inventors: JIYONG CHOI, Yongin-si (KR); YOUNGJIN PARK, Yongin-si (KR); HEECHOON AHN, Yongin-si (KR); JUHUI YUN, Yongin-si (KR); YESEUL LEE, Yongin-si (KR); SEOWON CHO, Yongin-si (KR); MORAN HA, Yongin-si (KR)

(21) Appl. No.: 18/409,664

(22) Filed: Jan. 10, 2024

(30) Foreign Application Priority Data
Apr. 14, 2023 (KR) 10-2023-0049167

(51) Int. Cl. H10K 85/40; C07B 59/00; C09K 11/06; H10K 50/15; H10K 85/30; H10K 85/60

(52) U.S. Cl. CPC H10K 85/40; C07B 59/004; C09K 11/06; H10K 50/15; H10K 85/346; H10K 85/654; H10K 85/6572; H10K 85/6574; C07B 2200/05; H10K 2101/20

(57) ABSTRACT

A light emitting element includes a first electrode, a second electrode on the first electrode, and at least one functional layer between the first electrode and the second electrode. The at least one functional layer includes a first compound represented by Formula 1.

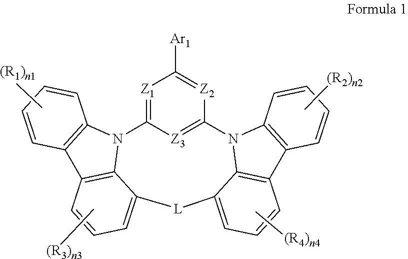

### CROSS-REFERENCE TO RELATED APPLICATIONS

The present application claims priority to and the benefit of Korean Patent Application No. 10-2023-0049167, filed on Apr. 14, 2023, in the Korean Intellectual Property Office, the entire content of which is incorporated herein by reference.

### BACKGROUND

### 1. Field

One or more embodiments of the present disclosure relates to a light emitting element and a hetero compound utilized therein.

### 2. Description of the Related Art

As image display devices, organic electroluminescence display devices and/or the like have been actively developed lately. Unlike liquid crystal display devices, and/or the like, the organic electroluminescence display devices are self-luminescent display devices in which holes and electrons respectively injected from a first electrode and a second electrode recombine in an emission layer of the organic electroluminescence display device, and thus a light emitting material including an organic compound in the emission layer emits light to accomplish display (e.g., of an image).

For application of organic electroluminescence elements to display devices, there is a demand and desire for organic electroluminescence elements having a low driving voltage, high light emitting efficiency, and a long life, and thus the development of materials, for organic electroluminescence elements, capable of stably attaining such characteristics is being continuously required and/or conducted.

In recent years, in order to obtain a highly efficient organic electroluminescence element, technologies pertaining to phosphorescence emission utilizing triplet state energy or fluorescence emission utilizing triplet-triplet annihilation (TTA) in which singlet excitons are generated through the collision of triplet excitons have been particularly researched and/or developed, and thermally activated delayed fluorescence (TADF) materials utilizing a delayed fluorescence phenomenon are under development.

### SUMMARY

One or more aspects of embodiments of the present disclosure are directed toward a light emitting element having increased light emitting efficiency and relatively longer element service life.

One or more aspects of embodiments of the present disclosure are directed toward a hetero compound capable of improving light emitting efficiency and element service life of a light emitting element.

Additional aspects will be set forth in part in the description which follows and, in part, will be apparent from the description, or may be learned by practice of the presented embodiments.

According to one or more embodiments of the present disclosure, a light emitting element includes a first electrode, a second electrode facing the first electrode, and at least one functional layer between the first electrode and the second electrode, wherein the at least one functional layer includes a first compound represented by Formula 1.

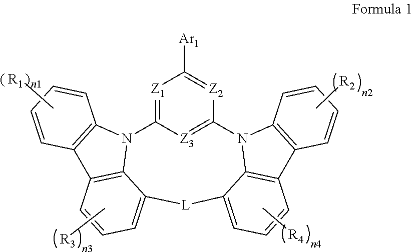

In Formula 1, Z₁ to Z₃ may each independently be N or CR₅, and at least one selected from among Z₁ to Z₃ is N, R₁ to R₅ may each independently be hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, Ar₁ may be hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, L may be a substituted or unsubstituted divalent oxy group, a substituted or unsubstituted divalent thio group, a substituted or unsubstituted divalent silyl group, a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, n1 and n2 may each independently be an integer of 0 to 4, and n3 and n4 may each independently be an integer of 0 to 3.

In one or more embodiments, the at least one functional layer may include a hole transport region on the first electrode, an emission layer on the hole transport region, and an electron transport region between the emission layer and the second electrode.

In one or more embodiments, the emission layer may be to emit phosphorescence or delayed fluorescence and may include the first compound.

In one or more embodiments, the emission layer may be to emit light having a central emission wavelength of about 430 nm to about 490 nm.

In one or more embodiments, the hole transport region may include the first compound.

In one or more embodiments, L may be represented by any one selected from among Formulas 2-1 to 2-4.

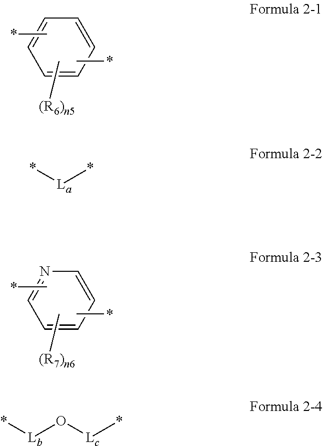

In Formulas 2-1 to 2-4, R₆ and R₇ may each independently be hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, Lₐ to L_{c} may each independently be a substituted or unsubstituted alkylene group having 1 to 5 carbon atoms, n5 may be an integer of 0 to 4, and n6 may be an integer of 0 to 3.

In one or more embodiments, the first compound represented by Formula 1 may be represented by Formula 1-1 or Formula 1-2.

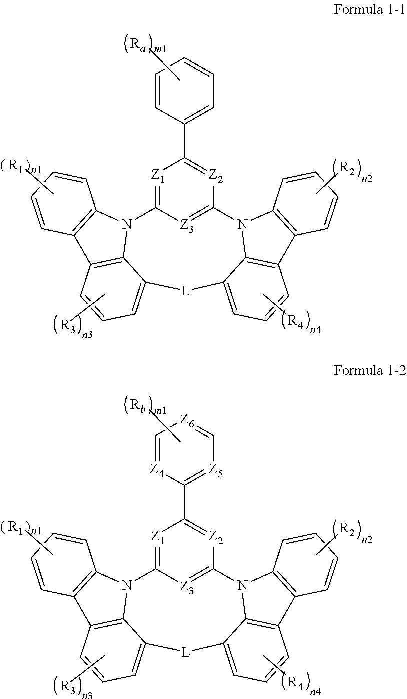

In Formulas 1-1 and 1-2, Z₄ to Z₆ may each independently be N or CR_{c}, and at least one selected from among Z₄ to Z₆ is N, Ra to Re may each independently be hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, m1 may be an integer of 0 to 5, m2 may be an integer of 0 to 2.

In Formulas 1-1 and 1-2, Z₁ to Z₃, R₁ to R₄, n1 to n4, and L may be the same as defined in Formula 1.

In one or more embodiments, Z₁ to Z₃ may all (e.g., each) be N.

In one or more embodiments, Ar₁ may be represented by any one selected from among Formulas 3-1 to 3-5.

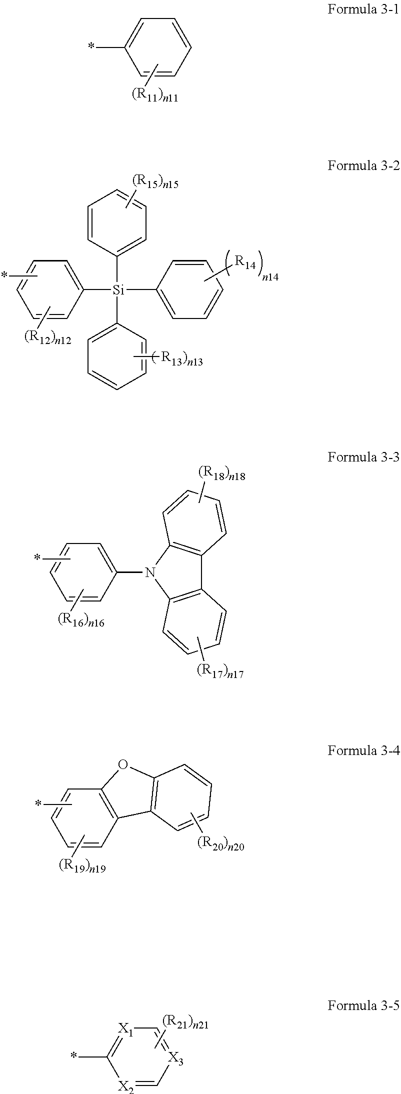

In Formulas 3-1 to 3-5, X₁ to X₃ may each independently be N or CR₂₂, and at least one selected from among X₁ to X₃ may be N, R₁₁ to R₂₂ may each independently be hydrogen, deuterium, a halogen, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, n11, and n13 to n15 may each independently be an integer of 0 to 5, n12, n16 to n18, and n20 may each independently be an integer of 0 to 4, n19 may be an integer of 0 to 3, and n21 may be an integer of 0 to 2.

In one or more embodiments, R₁ to R₄ may each independently be hydrogen or deuterium.

In one or more embodiments, L may be represented by any one selected from among Formulas 2-5 to 2-8.

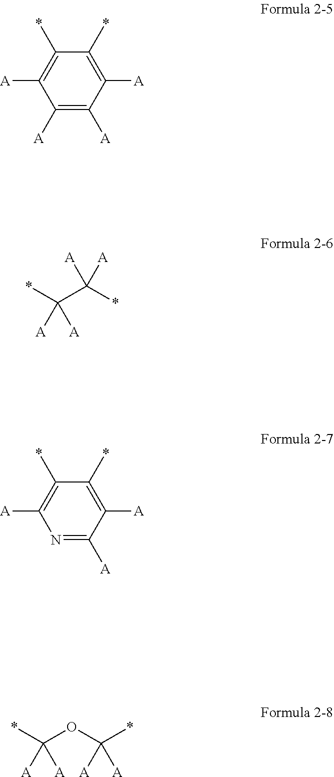

In Formulas 2-5 to 2-8, A may be hydrogen or deuterium.

In one or more embodiments, the emission layer may further include a second compound represented by Formula HT-1.

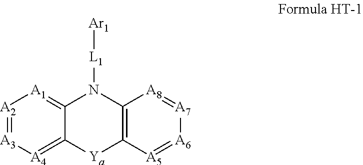

In Formula HT-1, A₁ to A₈ may each independently be N or CR₅₁, L₁ may be a direct linkage, a substituted or unsubstituted arylene group having 6 to 30 ring-forming carbon atoms, or a substituted or unsubstituted heteroarylene group having 2 to 30 ring-forming carbon atoms, Yₐ may be a direct linkage, CR₅₂R₅₃, or SiR₅₄R₅₅, Ar₁ may be a substituted or unsubstituted aryl group having 6 to 30 ring-forming carbon atoms, or a substituted or unsubstituted heteroaryl group having 2 to 30 ring-forming carbon atoms, and R₅₁ to R₅₅ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 ring-forming carbon atoms, a substituted or unsubstituted aryl group having 6 to 60 ring-forming carbon atoms, or a substituted or unsubstituted heteroaryl group having 2 to 60 ring-forming carbon atoms, and/or bonded to an adjacent group to form a ring.

In one or more embodiments, the emission layer may further include a third compound represented by Formula D-1.

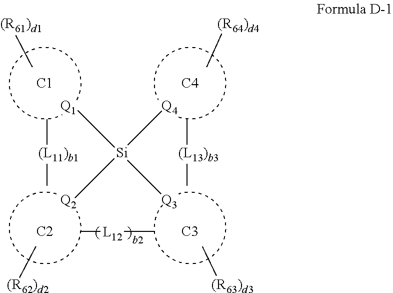

In Formula D-1 above, Q₁ to Q₄ may each independently be C or N, C1 to C4 may each independently be a substituted or unsubstituted hydrocarbon ring having 5 to 30 ring-forming carbon atoms, or a substituted or unsubstituted heterocycle having 2 to 30 ring-forming carbon atoms, L11 to L13 may each independently be a direct linkage,

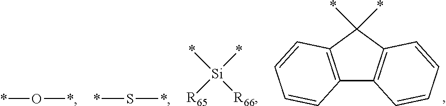

a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, b to b3 may each independently be 0 or 1, R₆₁ to R₆₆ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 ring-forming carbon atoms, a substituted or unsubstituted aryl group having 6 to 60 ring-forming carbon atoms, or a substituted or unsubstituted heteroaryl group having 2 to 60 ring-forming carbon atoms, and/or bonded to an adjacent group to form a ring, and d1 to d4 may each independently be an integer of 0 to 4.

In one or more embodiments of the present disclosure, an electronic device is at least one selected from among large-sized display devices such as televisions, monitors, and outdoor billboards, and small- and medium-sized display devices such as personal computers, laptop computers, personal digital terminals, vehicle display devices, game consoles, portable electronic devices, and cameras. The electronic device includes at least one light emitting element, and the emission layer of the at least one light emitting element includes a first compound represented by Formula 1.

In one or more embodiments of the present disclosure, a hetero compound is represented by Formula 1.

### BRIEF DESCRIPTION OF THE DRAWINGS

The accompanying drawings are included to provide a further understanding of the present disclosure, and are incorporated in and constitute a part of the present disclosure. The drawings illustrate embodiments of the present disclosure and, together with the description, serve to explain principles of the present disclosure. Above and/or other aspects of the present disclosure should become apparent and appreciated from the following description of embodiments taken in conjunction with the accompanying drawings. In the drawings:

FIG. 1 is a plan view of a display device according to one or more embodiments of the present disclosure;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 and FIG. 8 are each a cross-sectional view of a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure; and

FIG. 11 is a view showing a vehicle in which a display device according to one or more embodiments is disposed.

### DETAILED DESCRIPTION

The present disclosure may be modified in one or more suitable manners and have many forms, and thus specific embodiments will be exemplified in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are utilized for referring to like elements. In the accompanying drawings, the dimensions of each structure may be exaggeratingly illustrated for clarity of the present disclosure. It will be understood that, although the terms “first,” “second,” etc., may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure. As utilized herein, the singular forms, “a,” “an,” and “the” are intended to include the plural forms as well, unless the context clearly indicates otherwise. Further, the utilization of “may” when describing embodiments of the present disclosure refers to “one or more embodiments of the present disclosure”.

In the present disclosure, it will be understood that the terms “comprise(s),” “include(s),” “have/has,” and/or the like specify the presence of features, numbers, steps, operations, component, parts, or combinations thereof disclosed in the present disclosure, but do not exclude the possibility of presence or addition of one or more other features, numbers, steps, operations, component, parts, or combinations thereof. As utilized herein, the terms “and,” “or,” and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, when a layer, a film, a region, or a plate is referred to as being “on” or “in an upper portion of” another layer, film, region, or plate, it may be not only “directly on” the layer, film, region, or plate, but intervening layers, films, regions, or plates may also be present. Opposite this, when a layer, a film, a region, or a plate is referred to as being “below”, “in a lower portion of” another layer, film, region, or plate, it can be not only directly under the layer, film, region, or plate, but intervening layers, films, regions, or plates may also be present. In some embodiments, it will be understood that when a part is referred to as being “on” another part, it can be disposed above the other part, or disposed under the other part as well. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, the term “substituted or unsubstituted” may refer to substituted or unsubstituted with at least one substituent selected from the group consisting of deuterium, a halogen, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified above may be substituted or unsubstituted. For example, a biphenyl group may be interpreted as an aryl group or a phenyl group substituted with a phenyl group.

In the present disclosure, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may each be monocyclic or polycyclic. In some embodiments, the rings formed by two adjacent groups being bonded to each other may be connected to another ring to form a spiro structure.

In the present disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the present disclosure, examples of the halogen may include fluorine, chlorine, bromine, or iodine.

In the present disclosure, an alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments of the present disclosure are not limited thereto.

In the present disclosure, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments of the present disclosure are not limited thereto.

In the present disclosure, an alkenyl group may refer to a hydrocarbon group including at least one carbon-carbon double bond in the middle or terminal of an alkyl group having 2 or more carbon atoms. The alkenyl group may be linear or branched. The number of carbon atoms in the alkenyl group is not specifically limited, for example, may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, etc., but embodiments of the present disclosure are not limited thereto.

In the present disclosure, an alkynyl group may refer to a hydrocarbon group including at least one carbon-carbon triple bond in the middle or terminal of an alkyl group having 2 or more carbon atoms. The alkynyl group may be linear or branched. Although the number of carbon atoms is not specifically limited, it may be 2 to 30, 2 to 20, or 2 to 10. Examples of the alkynyl group may include an ethynyl group, a propynyl group, etc., but embodiments of the present disclosure are not limited thereto.

In the present disclosure, the hydrocarbon ring group may refer to any functional group or substituent derived from an aliphatic hydrocarbon ring. The hydrocarbon ring group may be a saturated hydrocarbon ring group having 5 to 20 ring-forming carbon atoms.

In the present disclosure, an aryl group may refer to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments of the present disclosure are not limited thereto.

In the present disclosure, the fluorenyl group may be substituted, and two substituents may be bonded to each other to form a spiro structure. Examples of the substituted fluorenyl group are as follows. However, embodiments of the present disclosure are not limited thereto.

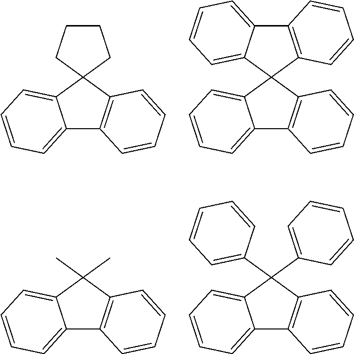

A heterocyclic group utilized herein may refer to any functional group or substituent derived from a ring containing at least one of B, O, N, P, Si, or Se as a heteroatom. The heterocyclic group may include an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

In the present disclosure, the heterocyclic group may contain at least one of B, O, N, P, Si or S as a heteroatom. When the heterocyclic group contains two or more heteroatoms, the two or more heteroatoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 30, 2 to 20, or 2 to 10.

In the present disclosure, the aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments of the present disclosure are not limited thereto.

In the present disclosure, the heteroaryl group may contain at least one of B, O, N, P, Si, or S as a heteroatom. When the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments of the present disclosure are not limited thereto.

In the present disclosure, the above description of the aryl group may be applied to an arylene group except that the arylene group is a divalent group. The above description of the heteroaryl group may be applied to a heteroarylene group except that the heteroarylene group is a divalent group.

In the present disclosure, the silyl group may include an alkylsilyl group and/or an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments of the present disclosure are not limited thereto.

In the present disclosure, the number of ring-forming carbon atoms in a carbonyl group is not specifically limited, for example, may be 1 to 40, 1 to 30, or 1 to 20. For example, the carbonyl group may have the following structures, but embodiments of the present disclosure are not limited thereto.

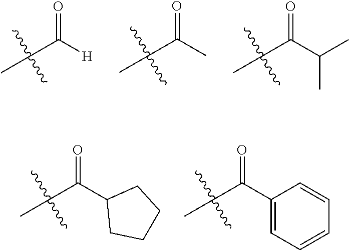

In the present disclosure, the number of carbon atoms in a sulfinyl group or a sulfonyl group is not particularly limited, for example, may be 1 to 30. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkylthio group and/or an arylthio group. The thio group may refer to that a sulfur atom is bonded to the alkyl group or the aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments of the present disclosure are not limited thereto.

In the present disclosure, an oxy group may refer to that an oxygen atom is bonded to the alkyl group or the aryl group as defined above. The oxy group may include an alkoxy group and/or an aryl oxy group. The alkoxy group may be a linear chain, a branched chain, or a ring. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc., but embodiments of the present disclosure are not limited thereto.

A boron group as utilized herein may refer to that a boron atom is bonded to the alkyl group or the aryl group as defined above. The boron group may include an alkyl boron group and/or an aryl boron group. Examples of the boron group may include a dimethylboron group, a trimethylboron group, a t-butyldimethylboron group, a diphenylboron group, a phenylboron group, etc., but embodiments of the present disclosure are not limited thereto.

In the present disclosure, the number of carbon atoms in an amine group is not specifically limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, etc., but embodiments of the present disclosure are not limited thereto.

In the present disclosure, the alkyl group among an alkoxy group, an alkylthio group, an alkylsulfoxy group, an alkylaryl group, an alkylamino group, an alkyl boron group, an alkyl silyl group, and an alkyl amine group may be the same as the examples of the alkyl group described above.

In the present disclosure, the aryl group among an aryloxy group, an arylthio group, an arylsulfoxy group, an arylamino group, an arylboron group, an arylsilyl group, and an arylamine group may be the same as the examples of the aryl group described above.

In the present disclosure, a direct linkage may refer to a single bond.

In the present disclosure, “ [symbol] ” and “ [symbol] ” may refer to a position to be connected.

Hereinafter, embodiments of the present disclosure will be described with reference to the accompanying drawings. In the present disclosure, term “light emitting device” may be utilized interchangeably with the term “light emitting element.”

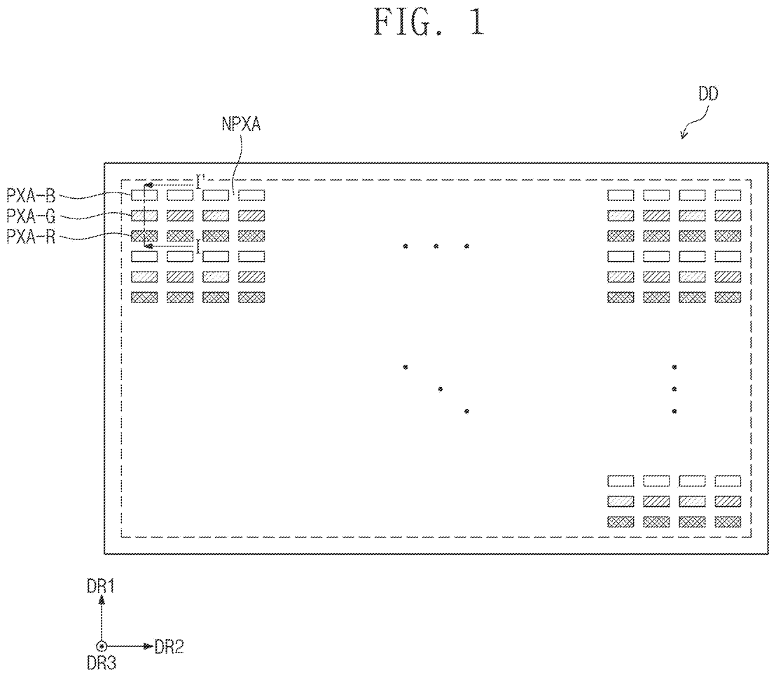

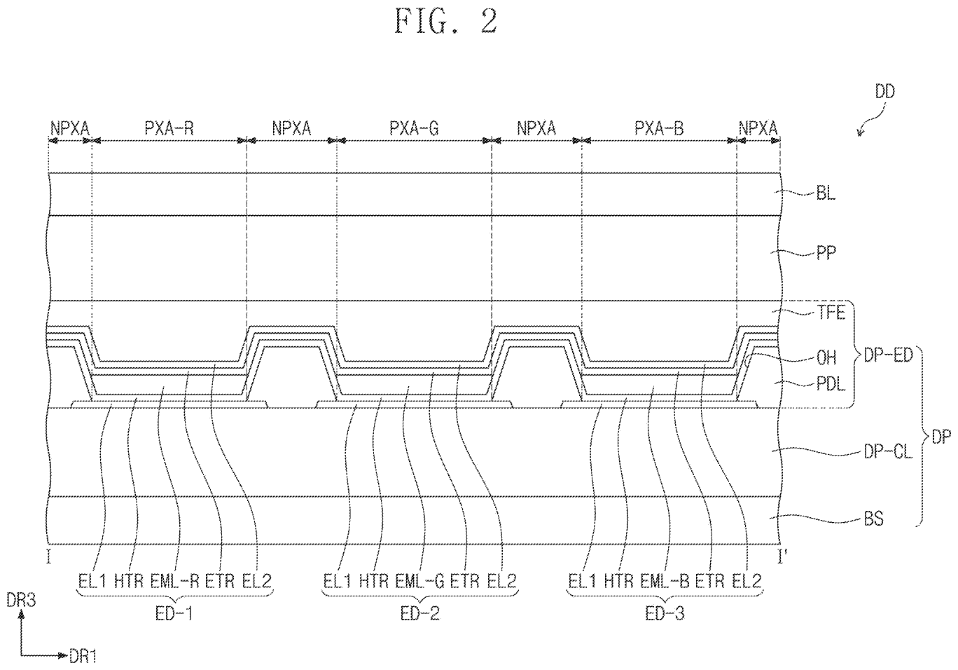

FIG. 1 is a plan view illustrating a display apparatus DD according to one or more embodiments of the present disclosure. FIG. 2 is a cross-sectional view of the display apparatus DD according to one or more embodiments of the present disclosure. FIG. 2 is a cross-sectional view illustrating a part taken along line I-I′ of FIG. 1.

The display apparatus DD may include a display panel DP and an optical layer PP disposed on the display panel DP. The display panel DP may include light emitting devices ED-1, ED-2, and ED-3. The display apparatus DD may include a plurality of light emitting devices ED-1, ED-2, and ED-3. The optical layer PP may be disposed on the display panel DP to control reflected light in the display panel DP due to external light. The optical layer PP may include, for example, a polarization layer and/or a color filter layer. In some embodiments, the optical layer PP may not be provided in the display apparatus DD.

A base substrate BL may be provided and disposed on the optical layer PP. The base substrate BL may be a member which provides a base surface on which the optical layer PP disposed. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may not be provided.

The display apparatus DD according to one or more embodiments may further include a filling layer. The filling layer may be disposed between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and a display device layer DP-ED. The display device layer DP-ED may include a pixel defining film PDL, the light emitting devices ED-1, ED-2, and ED-3 disposed between respective portions of the pixel defining film PDL, and an encapsulation layer TFE disposed on the light emitting devices ED-1, ED-2, and ED-3.

The base layer BS may be a member which provides a base surface on which the display device layer DP-ED is disposed. The base layer BS may be a glass substrate, a metal substrate, a plastic substrate, etc. However, embodiments of the present disclosure are not limited thereto, and the base layer BS may be an inorganic layer, an organic layer, or a composite material layer.

In one or more embodiments, the circuit layer DP-CL may be disposed on the base layer BS, and the circuit layer DP-CL may include a plurality of transistors. Each of the transistors may include a control electrode, an input electrode, and an output electrode. For example, in some embodiments, the circuit layer DP-CL may include switching transistor(s) and driving transistor(s) for driving the light emitting devices ED-1, ED-2, and ED-3 of the display device layer DP-ED.

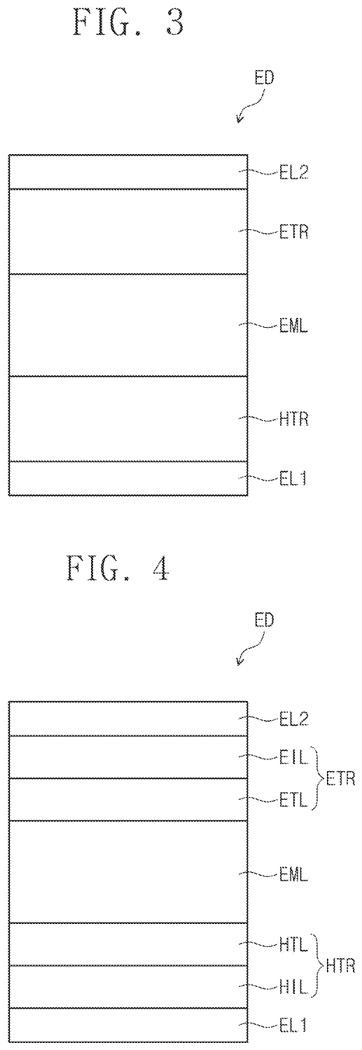

Each of the light emitting devices ED-1, ED-2, and ED-3 may independently have a structure of one of light emitting devices ED of embodiments according to FIGS. 3 to 6, which will be described later. Each of the light emitting devices ED-1, ED-2, and ED-3 may include a first electrode EL1, a hole transport region HTR, respective emission layers EML-R, EML-G, and EML-B, an electron transport region ETR, and a second electrode EL2.

FIG. 2 illustrates an embodiment in which the respective emission layers EML-R, EML-G, and EML-B of the light emitting devices ED-1, ED-2, and ED-3 are disposed in openings OH defined in the pixel defining film PDL, and the hole transport region HTR, the electron transport region ETR, and the second electrode EL2 are provided as a common layer in the entire light emitting devices ED-1, ED-2, and ED-3. However, embodiments of the present disclosure are not limited thereto, and in some embodiments, the hole transport region HTR and the electron transport region ETR may be provided by being patterned inside the openings OH defined in the pixel defining film PDL. For example, in some embodiments, the hole transport region HTR, the respective emission layers EML-R, EML-G, and EML-B, and the electron transport region ETR of the light emitting devices ED-1, ED-2, and ED-3 may be provided by being patterned in an inkjet printing method.

The encapsulation layer TFE may cover the light emitting devices ED-1, ED-2, and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE may include at least one insulation layer. In some embodiments, the encapsulation layer TFE may include at least one inorganic film (hereinafter, an encapsulation-inorganic film). In some embodiments, the encapsulation layer TFE may include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. The encapsulation-organic film may include an acrylic-based compound, an epoxy-based compound, and/or the like. In some embodiments, the encapsulation-organic film may include a photopolymerizable organic material, but embodiments of the present disclosure are not particularly limited thereto.

The encapsulation layer TFE may be disposed on the second electrode EL2 and may be disposed filling the opening OH.

Referring to FIG. 1 and FIG. 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light emitting devices ED-1, ED-2, and ED-3 is emitted. The light emitting regions PXA-R, PXA-G, and PXA-B may be spaced apart from each other on a plane.

Each of the light emitting regions PXA-R, PXA-G, and PXA-B may be a region divided by the pixel defining film PDL. The non-light emitting areas NPXA may be areas between adjacent light emitting areas PXA-R, PXA-G, and PXA-B, which correspond to the pixel defining film PDL. In some embodiments, the light emitting regions PXA-R, PXA-G, and PXA-B may respectively correspond to pixels. The pixel defining film PDL may divide the light emitting devices ED-1, ED-2, and ED-3. The respective emission layers EML-R, EML-G, and EML-B of the light emitting devices ED-1, ED-2, and ED-3 may be disposed in openings OH defined in the pixel defining film PDL and separated from each other.

The light emitting regions PXA-R, PXA-G, and PXA-B may be divided into a plurality of groups according to the color of light generated from the light emitting devices ED-1, ED-2, and ED-3. In the display apparatus DD of one or more embodiments illustrated in FIG. 1 and FIG. 2, three light emitting regions PXA-R, PXA-G, and PXA-B, which emit red light, green light, and blue light, respectively, are illustrated as an example. For example, in one or more embodiments, the display device DD may include a red light emitting region PXA-R, a green light emitting region PXA-G, a blue light emitting region PXA-B that are separated from each other.

In the display apparatus DD according to one or more embodiments, the plurality of light emitting devices ED-1, ED-2 and ED-3 may be to emit light beams having wavelengths different from each other. For example, in some embodiments, the display apparatus DD may include a first light emitting device ED-1 that emits red light, a second light emitting device ED-2 that emits green light, and a third light emitting device ED-3 that emits blue light. For example, in some embodiments, the red light emitting region PXA-R, the green light emitting region PXA-G, and the blue light emitting region PXA-B of the display apparatus DD may correspond to the first light emitting device ED-1, the second light emitting device ED-2, and the third light emitting device ED-3, respectively.

However, embodiments of the present disclosure are not limited thereto, and the first to third light emitting devices ED-1, ED-2, and ED-3 may be to emit light beams in substantially the same wavelength range or at least one light emitting device may be to emit a light beam in a wavelength range different from the others. For example, in some embodiments, the first to third light emitting devices ED-1, ED-2, and ED-3 may all emit blue light.

The light emitting regions PXA-R, PXA-G, and PXA-B in the display apparatus DD according to one or more embodiments may be arranged in a stripe form. Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B may be arranged with each other along the second directional axis DR2. In some embodiments, the red light emitting region PXA-R, the green light emitting region PXA-G, and the blue light emitting region PXA-B may be alternately arranged in this order along a first direction axis DR1.

FIG. 1 and FIG. 2 illustrate that all the light emitting regions PXA-R, PXA-G, and PXA-B have similar area, but embodiments of the present disclosure are not limited thereto. Thus, the light emitting regions PXA-R, PXA-G, and PXA-B may have different areas from each other according to the wavelength range of the emitted light. The areas of the light emitting regions PXA-R, PXA-G, and PXA-B may refer to areas when viewed on a plane defined by the first direction axis DR1 and the second direction axis DR2 (e.g., the areas in a plan view).

In some embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality required in the display apparatus DD. For example, in some embodiments, the arrangement form of the light emitting regions PXA-R, PXA-G, and PXA-B may be a pentile (PENTILE©) arrangement form (for example, an RGBG matrix, an RGBG structure, or RGBG matrix structure) or a diamond (Diamond Pixel™) arrangement form (e.g., a display (e.g., an OLED display) containing red, blue, and green (RGB) light-emitting regions arranged in the shape of diamonds). PENTILE© is a duly registered trademark of Samsung Display Co., Ltd. Diamond Pixel™ is a trademark of Samsung Display Co., Ltd.

In some embodiments, the areas of the light emitting regions PXA-R, PXA-G, and PXA-B may be different from each other. For example, in some embodiments, the area of the green light emitting region PXA-G may be smaller than that of the blue light emitting region PXA-B, but embodiments of the present disclosure are not limited thereto.

Hereinafter, FIG. 3 to FIG. 6 are each a cross-sectional view schematically showing a light emitting element according to one or more embodiments of the present disclosure. The light emitting element ED of one or more embodiments may include a first electrode EL1, a hole transport region HTR, an emission layer EML, an electron transport region ETR, and a second electrode EL2 stacked in order (e.g., in the stated order).

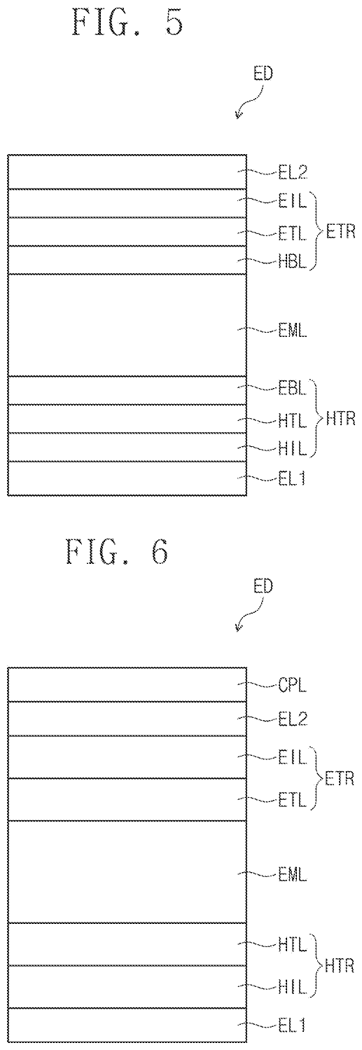

Compared with FIG. 3, FIG. 4 illustrates a cross-sectional view of a light emitting element ED of one or more embodiments, in which a hole transport region HTR may include a hole injection layer HIL and a hole transport layer HTL, and an electron transport region ETR may include an electron injection layer EIL and an electron transport layer ETL. In some embodiments, compared with FIG. 3, FIG. 5 illustrates a cross-sectional view of a light emitting element ED, in which a hole transport region HTR may include a hole injection layer HIL, a hole transport layer HTL, and an electron blocking layer EBL, and an electron transport region ETR may include an electron injection layer EIL, an electron transport layer ETL, and a hole blocking layer HBL. Compared with FIG. 4, FIG. 6 illustrates a cross-sectional view of a light emitting element ED of one or more embodiments including a capping layer CPL disposed on a second electrode EL2.

The first electrode EL1 has conductivity (e.g., is a conductor). The first electrode EL1 may be formed of a metal material, a metal alloy, or a conductive compound. The first electrode EL1 may be an anode or a cathode. However, embodiments of the present disclosure are not limited thereto. In some embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W), indium (In), tin (Sn), and zinc (Zn), one or more compounds each being of two or more selected therefrom, one or more mixtures each being of two or more selected therefrom, and/or one or more oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In some embodiments, the first electrode EL1 may have a multilayer structure including a reflective film or a transflective film formed of the above-described materials, and a transparent conductive film formed of ITO, IZO, ZnO, ITZO, etc. For example, in some embodiments, the first electrode EL1 may have a three-layer structure of ITO/Ag/ITO, but embodiments of the present disclosure are not limited thereto. The first electrode EL1 may include one of the above-described metal materials, combinations of at least two metal materials of the above-described metal materials, oxides of the above-described metal materials, and/or the like. A thickness of the first electrode EL1 may be from about 700 Å to about 10,000 Å. For example, in some embodiments, the thickness of the first electrode EL1 may be from about 1,000 Å to about 3,000 Å.

The hole transport region HTR may be provided on the first electrode EL1. The hole transport region HTR may include at least one of a hole injection layer HIL, a hole transport layer HTL, a buffer layer or an emission-auxiliary layer, or an electron blocking layer EBL. A thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, in one or more embodiments, the hole transport region HTR may have a single layer structure of a hole injection layer HIL or a hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order (e.g., in the stated order) from the first electrode EL1, but embodiments of the present disclosure are not limited thereto.

The hole transport region HTR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the hole transport region HTR may include a compound represented by Formula H-1:

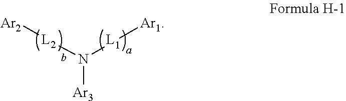

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In some embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. In some embodiments, the compound represented by Formula H-1 may be a diamine compound in which at least one selected from among Ar₁ to Ar₃ may include an amine group as a substituent. In some embodiments, the compound represented by Formula H-1 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be any one selected from among compounds in Compound Group H. However, the compounds listed in Compound Group H are mere examples, and the compounds represented by Formula H-1 are not limited to those represented in Compound Group H:

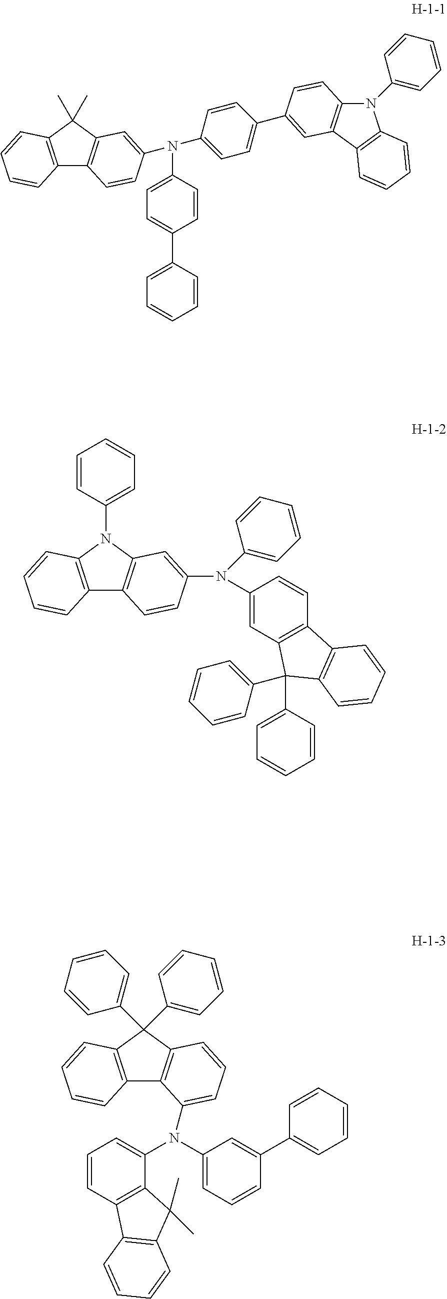 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

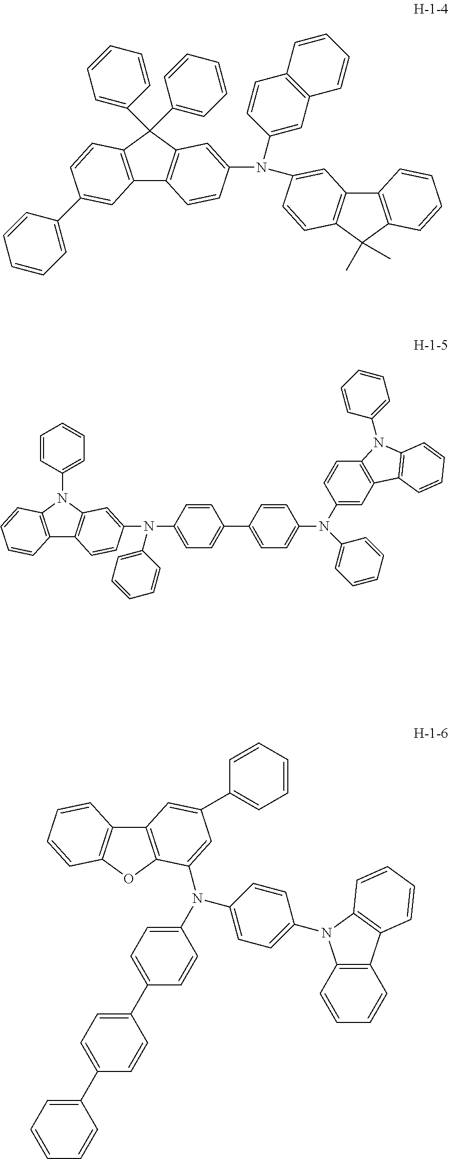 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

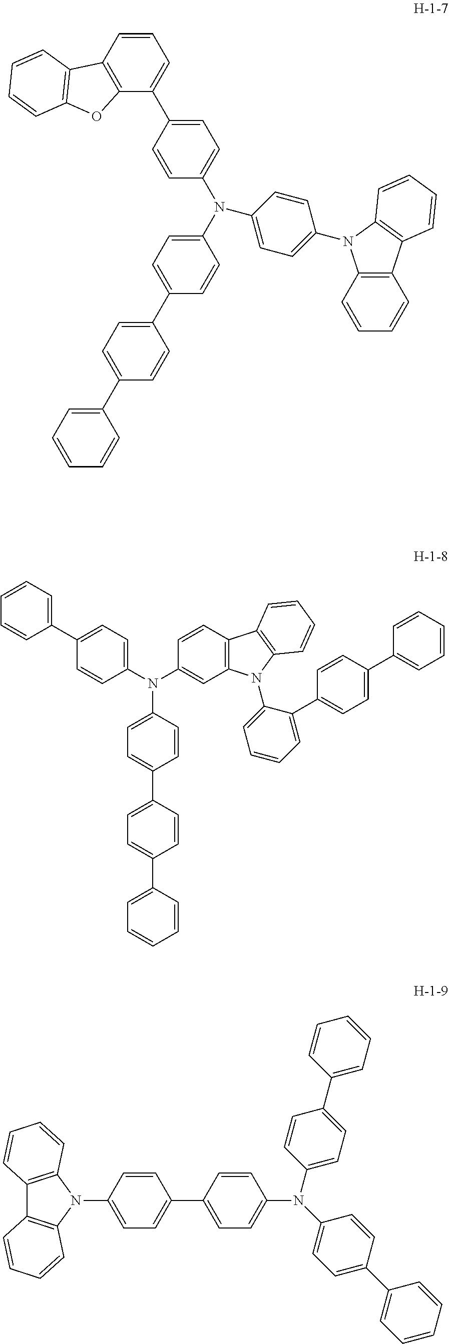 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

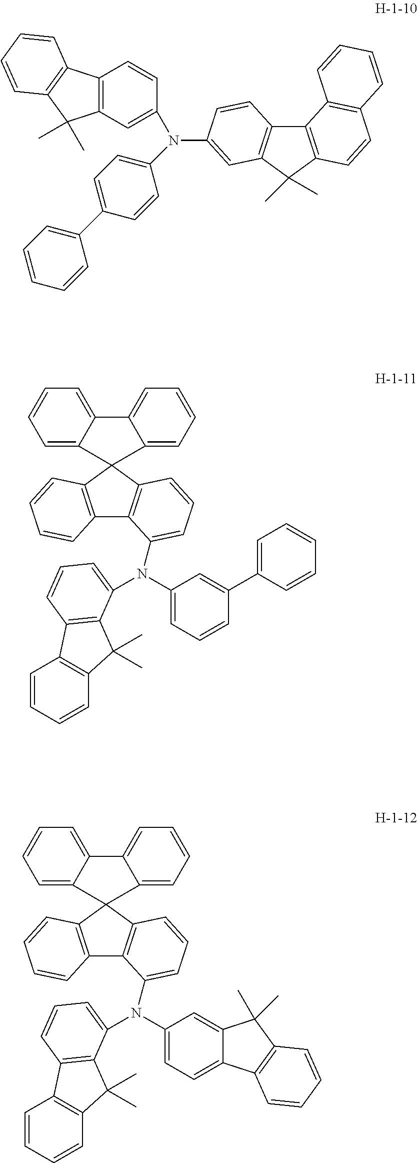 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21]

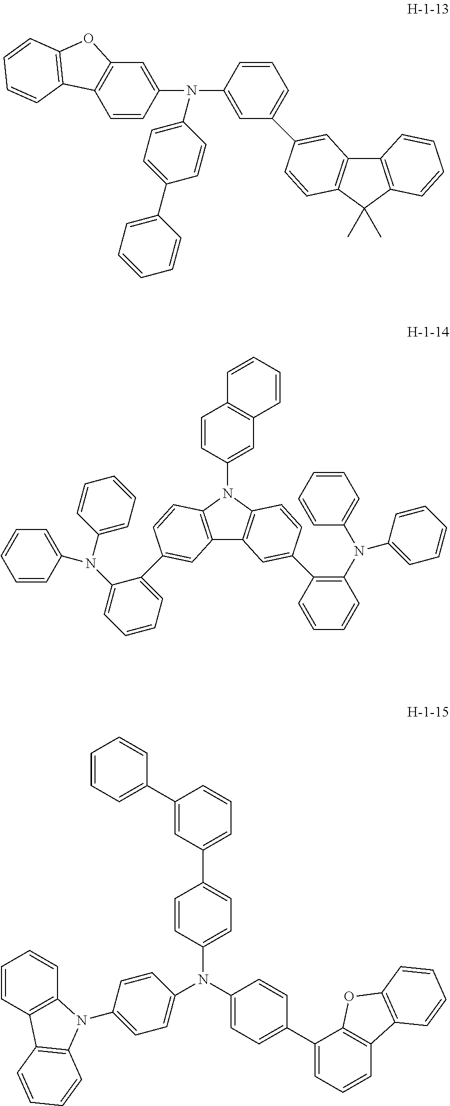 [SMILES: CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

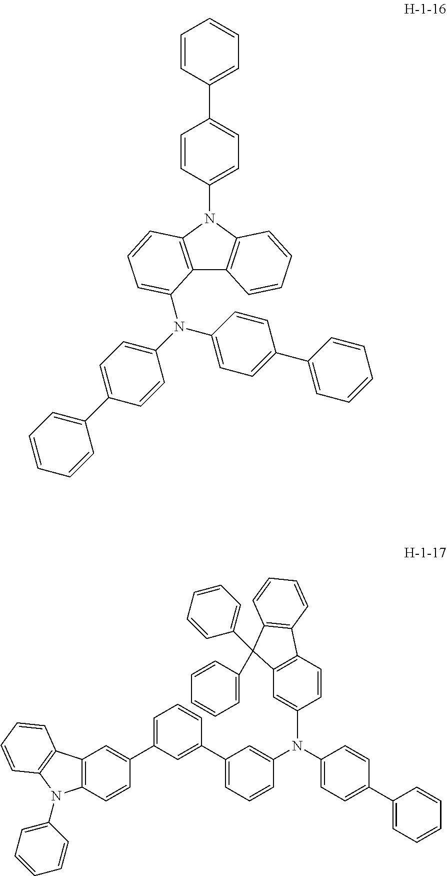 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1]

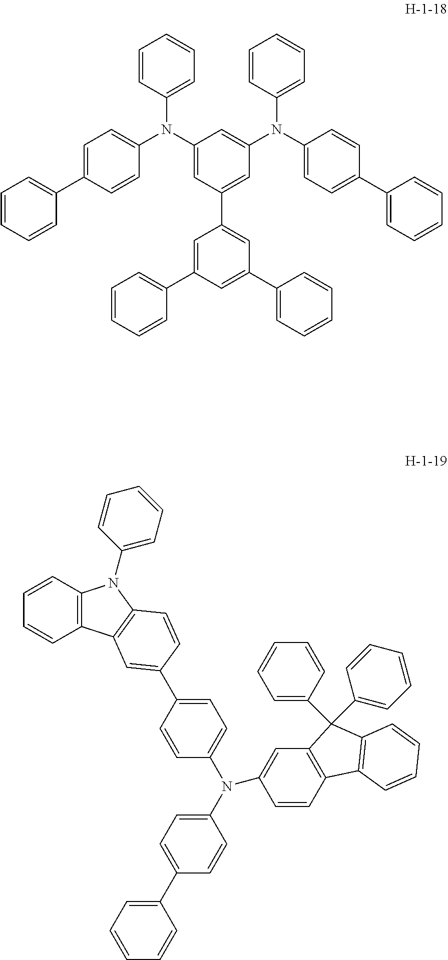 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In one or more embodiments, the hole transport region HTR may include at least one selected from among a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

In one or more embodiments, the hole transport region HTR may include at least one selected from among a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalen-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may include at least one selected from among 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include at least one of the above-described compounds of the hole transport region in at least one of a hole injection layer HIL, a hole transport layer HTL, or an electron blocking layer EBL.

A thickness of the hole transport region HTR may be from about 100 Å to about 10,000 Å, for example, from about 100 Å to about 5,000 Å. When the hole transport region HTR includes a hole injection layer HIL, the hole injection layer HIL may have, for example, a thickness of about 30 Å to about 1,000 Å. When the hole transport region HTR includes a hole transport layer HTL, the hole transport layer HTL may have a thickness of about 250 Å to about 1,000 Å. For example, when the hole transport region HTR includes an electron blocking layer EBL, the electron blocking layer EBL may have a thickness of about 10 Å to about 1,000 Å. When the thicknesses of the hole transport region HTR, the hole injection layer HIL, the hole transport layer HTL, and the electron blocking layer EBL satisfy the above-described ranges, satisfactory hole transport properties may be achieved without a substantial increase in driving voltage.

In one or more embodiments, the hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but embodiments of the present disclosure are not limited thereto. For example, in some embodiments, the p-dopant may include at least one selected from among a metal halide compound such as CuI and/or RbI, a quinone derivative such as tetracyanoquinodimethane (TCNQ) and/or 2,3,5,6-tetrafluoro-7,7,8,8-tetracyanoquinodimethane (F4-TCNQ), a metal oxide such as tungsten oxide and/or molybdenum oxide, a cyano group-containing compound such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but embodiments of the present disclosure are not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be included in the hole transport region HTR may be utilized as a material to be included in the buffer layer. The electron blocking layer EBL is a layer that serves to prevent or reduce the electron injection from the electron transport region ETR to the hole transport region HTR.

The emission layer EML may be provided on the hole transport region HTR. The emission layer EML may have, for example, a thickness of about 100 Å to about 1000 Å or about 100 Å to about 300 Å. The emission layer EML may have a single layer formed of a single material, a single layer formed of a plurality of different materials, or a multilayer structure having a plurality of layers formed of a plurality of different materials.

The light emitting element ED of one or more embodiments may include a hetero compound represented by Formula 1 in at least one functional layer disposed between the first electrode EL1 and the second electrode EL2. In the light emitting element ED according to one or more embodiments, the emission layer EML may include a hetero compound of one or more embodiments. In one or more embodiments, the emission layer EML may include the hetero compound of one or more embodiments as a host material. The hetero compound of one or more embodiments may be a host material of the emission layer EML. In the present disclosure, the hetero compound of one or more embodiments, which will be described later, may be referred to as a first compound.

The hetero compound of one or more embodiments may include one nitrogen-containing heterocycle and two carbazole moieties connected to the nitrogen-containing heterocycle, and the two carbazole moieties are linked through a linker. For example, the hetero compound of one or more embodiments may include a structure in which a hexagonal (e.g., a six-membered) heterocycle containing 1 to 3 nitrogen atoms as ring-forming atoms is a core structure, two carbazole moieties are connected to the hexagonal heterocycle, and the two carbazole moieties are bonded through a linker.

In the hetero compound of one or more embodiments, the linker connecting the two carbazole moieties may include a divalent oxy group, a divalent thio group, a divalent silyl group, an alkylene group, an arylene group, or a heteroarylene group. In one or more embodiments, the linker connecting the two carbazole groups may be a substituted or unsubstituted phenylene group, a substituted or unsubstituted divalent ethyl group, a substituted or unsubstituted pyridinylene group, or a substituted or unsubstituted divalent dimethyloxy group.

In some embodiments, each of the two carbazole moieties included in the hetero compound may be represented by Formula a. The linker connecting the two carbazole moieties may be connected to a position of carbon 4 or carbon 5 of Formula a. In the present disclosure, for convenience of description, each substituent substituted on the carbazole moiety is omitted and represented by a hydrogen, but substituents other than hydrogens may be substituted at each carbon position.

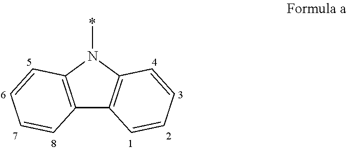

In Formula a, [symbol] is a position connected to the nitrogen-containing heterocycle described above.

In one or more embodiments, the hetero compound may be represented by Formula 1:

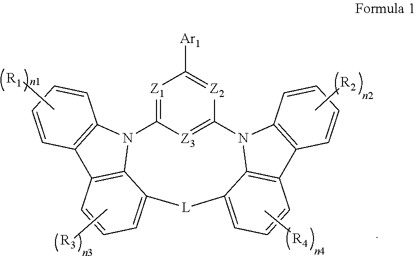

The hetero compound of one or more embodiments represented by Formula 1 may include a structure in which two carbazole moieties connected to a nitrogen-containing heterocycle are included and the two carbazole moieties are linked through a linker. In Formula 1, the hexagonal (e.g., six-membered) ring including Z₁ to Z₃ in the ring may correspond to the nitrogen-containing heterocycle described above, and the substituents substituted with substituents represented by R₁ to R₄ may correspond to the two carbazole moieties described above. The substituent represented by L may correspond to the linker connecting the two carbazole moieties described above.

In Formula 1, Z₁ to Z₃ may each independently be N or CR₅. In some embodiments, at least one selected from among Z₁ to Z₃ may be N, and the others may be CR₅. For example, in some embodiments, one selected from among Z₁ to Z₃ may be N, and the others may be CR₅. In some embodiments, two selected from among Z₁ to Z₃ may be N, and the other one may be CR₅. In some embodiments, in Formula 1, Z₁ to Z₃ may all be N.

In Formula 1, R₁ to R₅ may each independently be hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in one or more embodiments, R₁ to R₄ may each independently be hydrogen or deuterium. R₅ may be hydrogen or deuterium.

In Formula 1, Ar₁ may be hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in one or more embodiments, Ar₁ may be a substituted or unsubstituted phenyl group, a substituted or unsubstituted tetraphenylsilyl group, a substituted or unsubstituted N-phenylcarbazole group, or a substituted or unsubstituted dibenzofuranyl group.

In Formula 1, L may be a substituted or unsubstituted divalent oxy group, a substituted or unsubstituted divalent thio group, a substituted or unsubstituted divalent silyl group, a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms. For example, in one or more embodiments, L may be a substituted or unsubstituted phenylene group, a substituted or unsubstituted divalent ethyl group, a substituted or unsubstituted pyridinylene group, or a substituted or unsubstituted divalent dimethyloxy group.

In Formula 1, n1 and n2 may each independently be an integer of 0 to 4. In Formula 1, when n1 is 0, the hetero compound of one or more embodiments may not be substituted with R₁. When n1 is 4, and R₁'s are all hydrogens, the embodiments may be the same as when n1 is 0. When n1 is an integer of 2 or greater, R₁ provided in plurality may all be the same, or at least one of the plurality of R₁'s may be different. In Formula 1, when n2 is 0, the hetero compound of one or more embodiments may not be substituted with R₂. When n2 is 4, and R₂'s are all hydrogens, the embodiments may be the same as when n2 is 0. When n2 is an integer of 2 or greater, R₂ provided in plurality may all be the same, or at least one of the plurality of R₂'s may be different.

In Formula 1, n3 and n4 may each independently be an integer of 0 to 3. In Formula 1, when n3 is 0, the hetero compound of one or more embodiments may not be substituted with R₃. When n3 is 3, and R₃'s are all hydrogens, the embodiments may be the same as when n3 is 0. When n3 is an integer of 2 or greater, R₃ provided in plurality may all be the same, or at least one of the plurality of R₃'s may be different. In Formula 1, when n4 is 0, the hetero compound of one or more embodiments may not be substituted with R₄. When n4 is 3, and R₄'s are all hydrogens, the embodiments may be the same as when n4 is 0. When n4 is an integer of 2 or greater, R₄ provided in plurality may all be the same, or at least one of the plurality of R₄'s may be different.

In Formula 1, the linker represented by L may be represented by any one selected from among Formulas 2-1 to 2-4.

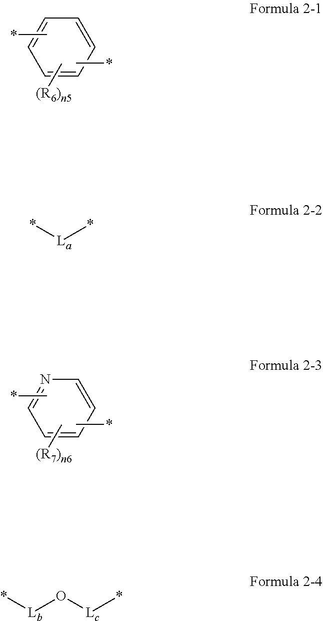

In Formulas 2-1 to 2-4, R₆ and R₇ may each independently be hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, in one or more embodiments, R₆ and R₇ may each independently be hydrogen or deuterium.

In Formulas 2-1 to 2-4, Lₐ to L_{c} may each independently be a substituted or unsubstituted alkylene group having 1 to 5 carbon atoms. For example, in one or more embodiments, Lₐ to L_{c} may each independently be a substituted or unsubstituted methylene group or a substituted or unsubstituted ethylene group.

In Formula 2-1, n5 is an integer of 0 to 4. In Formula 2-1, when n5 is 0, the hetero compound of one or more embodiments may not be substituted with R₆. When n5 is 4, and Re's are all hydrogens, the embodiments may be the same as when n5 is 0. When n5 is an integer of 2 or greater, R₆ provided in plurality may all be the same, or at least one of the plurality of R₆'s may be different.

In Formula 2-3, n6 is an integer of 0 to 3. In Formula 2-3, when n6 is 0, the hetero compound of one or more embodiments may not be substituted with R₇. When n6 is 3, and R₇'s are all hydrogens, the embodiments may be the same as when n6 is 0. When n6 is an integer of 2 or greater, R₇ provided in plurality may all be the same, or at least one of the plurality of R₇'s may be different.

In Formulas 2-1 to 2-4, “ [symbol] ” is a position connected other corresponding moieties of Formula 1. In Formulas 2-1 to 2-4, “ [symbol] ” is a position connected to one or the other of the two carbazole moieties indicated in Formula 1.

In one or more embodiments, in Formula 1, the linker represented by L may be represented by any one selected from among Formulas 2-5 to 2-8.

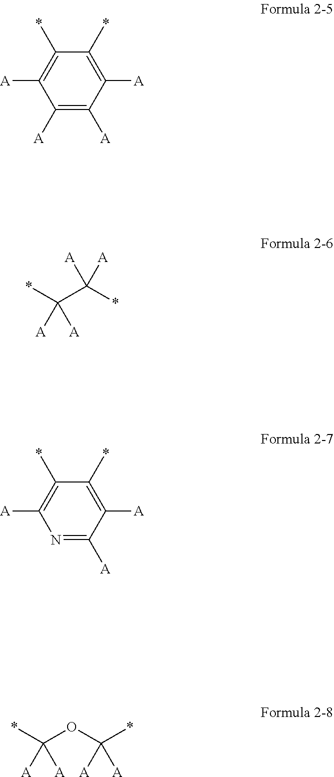

In Formulas 2-5 to 2-8, A may independently be hydrogen or deuterium. In some embodiments, the plurality of A's indicated in Formulas 2-5 to 2-8 may each be the same as or different from each other. For example, in some embodiments, the plurality of A's may all be hydrogens, the plurality of A's may all be deuterium, or some of the plurality of A's may be hydrogens and the others may be deuterium.

In some embodiments, in Formulas 2-5 to 2-8, “ [symbol] ” is a position connected to other corresponding moieties of Formula 1. In Formulas 2-5 to 2-8, “ [symbol] ” is a position connected to one or the other of the two carbazole moieties indicated in Formula 1.

In one or more embodiments, in Formula 1, the substituent represented by Ar₁ may be represented by any one selected from among Formulas 3-1 to 3-5.

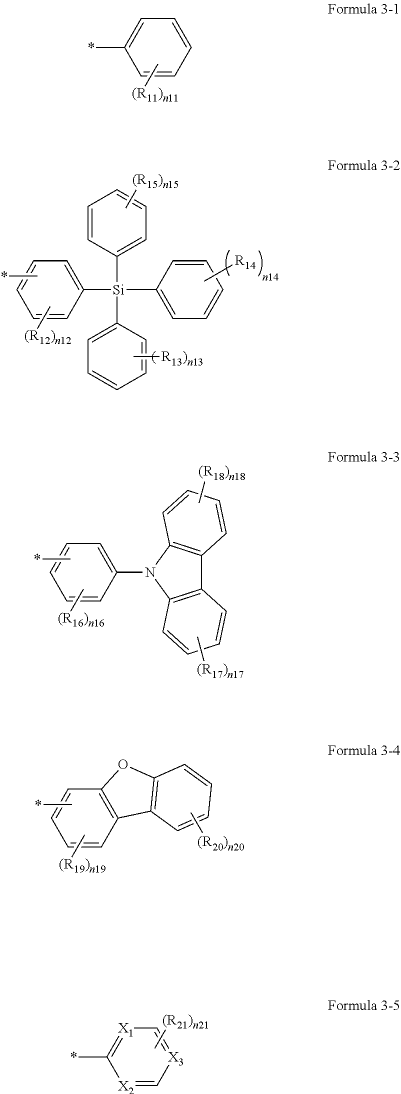

In Formula 3-5, X₁ to X₃ may each independently be N or CR₂₂. In some embodiments, at least one selected from among X₁ to X₃ may be N, and the others may be CR₂₂. For example, in some embodiments, one selected from among X₁ to X₃ may be N, and the other two may be CR₂₂. In some embodiments, two selected from among X₁ to X₃ may be N, and the other one may be CR₂₂. In some embodiments, in Formula 3-5, X₁ to X₃ may all be N.

In Formulas 3-1 to 3-5, R₁₁ to R₂₂ may each independently be hydrogen, deuterium, a halogen, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In some embodiments, one or more selected from R₁₁ to R₂₁ may each independently be bonded to an adjacent group to form a ring. For example, in some embodiments, R₁₁ to R₂₁ may each independently be hydrogen, deuterium, a substituted or unsubstituted silyl group, or a substituted or unsubstituted phenyl group. In some embodiments, R₁₄ and R₁₅ may be bonded to each other to form a ring.

In Formulas 3-1 and 3-2, n11, and n13 to n15 may each independently be an integer of 0 to 5. In Formula 3-1, when n11 is 0, the hetero compound of one or more embodiments may not be substituted with R₁₁. When n11 is 5, and R₁₁'s are all hydrogens, the embodiments may be the same as when n11 is 0. When n11 is an integer of 2 or greater, R₁₁ provided in plurality may all be the same, or at least one of the plurality of R₁₁'s may be different. In Formula 3-2, when n13 to n15 are each 0, the hetero compound of one or more embodiments may not be substituted with each of R₁₃ to R₁₅. When n13 to n15 are each 5 and R₁₃ to R₁₅ are each hydrogen, the embodiments may be the same as when n13 to n15 are each 0. When n13 to n15 are each an integer of 2 or greater, R₁₃ to R₁₅ provided in plurality may each be the same, or at least one of the plurality of R₁₃ to R₁₅ may be different.

In Formulas 3-2 to 3-4, n12, and n16 to n18, and n20 may each independently be an integer of 0 to 4. In Formula 3-2, when n12 is 0, the hetero compound of one or more embodiments may not be substituted with R₁₂. When n12 is 4, and R₁₂'s are all hydrogens, the embodiments may be the same as when n12 is 0. When n12 is an integer of 2 or greater, R₁₂ provided in plurality may all be the same, or at least one of the plurality of R₁₂'s may be different. In Formula 3-3, when n16 to n18 are each 0, the hetero compound of one or more embodiments may not be substituted with each of R₁₆ to R₁₈. When n16 to n18 are each 4 and R₁₆ to R₁₈ are each hydrogen, the embodiments may be the same as when n16 to n18 are each 0. When n16 to n18 are each an integer of 2 or greater, R₁₆ to R₁₈ provided in plurality may each be the same, or at least one of the plurality of R₁₆ to R₁₈ may be different. In Formula 3-4, when n20 is 0, the hetero compound of one or more embodiments may not be substituted with R₂₀. When n20 is 4, and R₂₀'s are all hydrogens, the embodiments may be the same as when n20 is 0. When n20 is an integer of 2 or greater, R₂₀ provided in plurality may all be the same, or at least one of the plurality of R₂₀'s may be different.

In Formula 3-4, n19 is an integer of 0 to 3. In Formula 3-4, when n19 is 0, the hetero compound of one or more embodiments may not be substituted with R₁₉. When n19 is 3, and R₁₉'s are hydrogens, the embodiments may be the same as when n19 is 0. When n19 is an integer of 2 or greater, R₁₉ provided in plurality may all be the same, or at least one of the plurality of R₁₉'s may be different.

In Formula 3-5, n21 is an integer of 0 to 2. In Formula 3-5, when n21 is 0, the hetero compound of one or more embodiments may not be substituted with R₂₁. When n21 is 2, and R₂₁'s are all hydrogens, the embodiments may be the same as when n21 is 0. When n21 is an integer of 2 or greater, R₂₁ provided in plurality may all be the same, or two R₂₁'s may be different.

In one or more embodiments, the hetero compound represented by Formula 1 may be represented by Formula 1-1 or Formula 1-2.

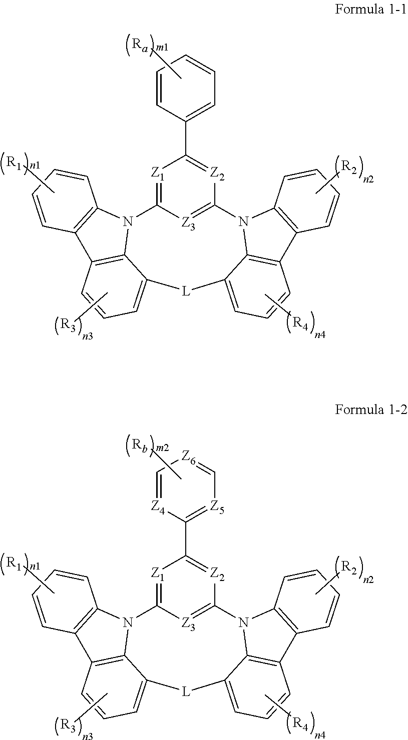

In Formula 1-2, Z₄ to Z₆ may each independently be N or CR_{c}. In some embodiments, at least one selected from among Z₄ to Z₆ may be N, and the others may be CR_{c}. For example, in some embodiments, one selected from among Z₄ to Z₆ may be N, and the others may be CR_{c}. In some embodiments, two selected from among Z₄ to Z₆ may be N, and the other one may be CR_{c}. In some embodiments, in Formula 1-2, Z₄ to Z₆ may all be N.

In Formulas 1-1 and 1-2, Ra to Re may each independently be hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In some embodiments, one or more selected from Ra to Re may each independently be bonded to an adjacent group to form a ring. For example, in some embodiments, Ra may be hydrogen, deuterium, a substituted or unsubstituted terphenylsilyl group, or a substituted or unsubstituted carbazole group. In some embodiments, Ra may be provided in plurality, one Ra may be an oxy group and the other Ra may be a phenyl group, and the oxy group and the phenyl group may be bonded to form a ring, thereby forming a dibenzofuran moiety. In some embodiments, Rb may be hydrogen or deuterium.

In Formula 1-1, m1 is an integer of 0 to 5. In Formula 1-1, when m1 is 0, the hetero compound of one or more embodiments may not be substituted with Ra. When m1 is 5, and Ra's are all hydrogens, the embodiments may be the same as when m1 is 0. When m1 is an integer of 2 or greater, Ra provided in plurality may all be the same, or at least one of the plurality of Ra's may be different.

In Formula 1-2, m2 is an integer of 0 to 2. In Formula 1-2, when m2 is 0, the hetero compound of one or more embodiments may not be substituted with Rb. When m2 is 2, and Rb's are all hydrogens atom, the embodiments may be the same as when m2 is 0. When m2 is an integer of 2 or greater, Rb provided in plurality may all be the same, or two Rb's may be different.

In Formulas 1-1 and 1-2, the same descriptions as in Formula 1 may also be applied to Z₁ to Z₃, R₁ to R₄, n1 to n4, and L.

In one or more embodiments, the hetero compound represented by Formula 1 may be represented by any one selected from among Formulas 1-3 to 1-12.

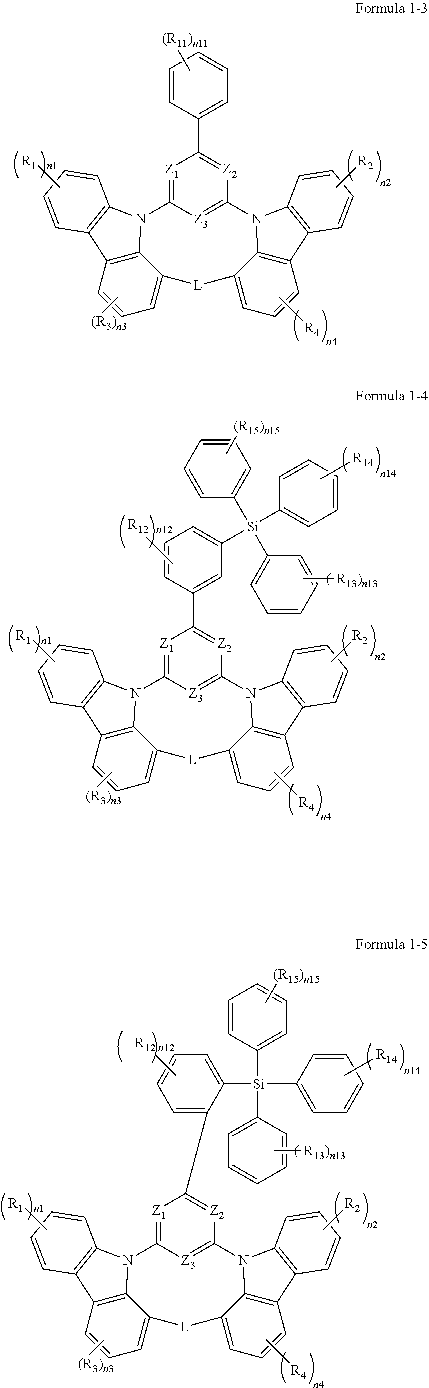

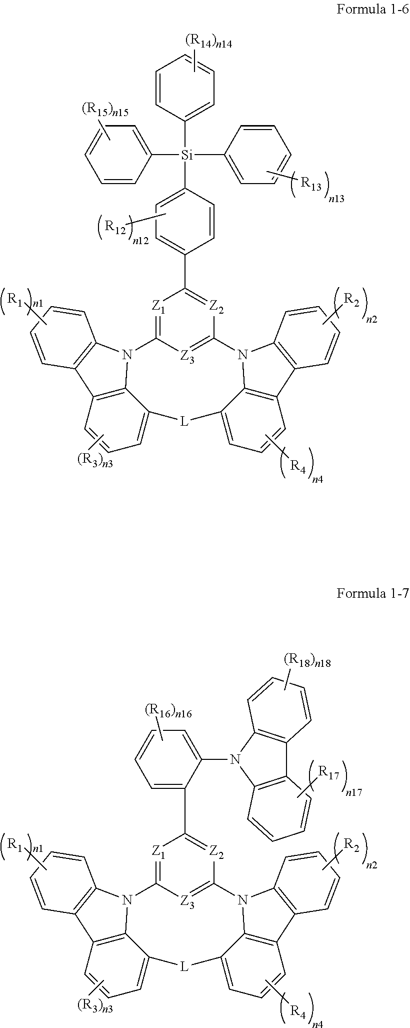

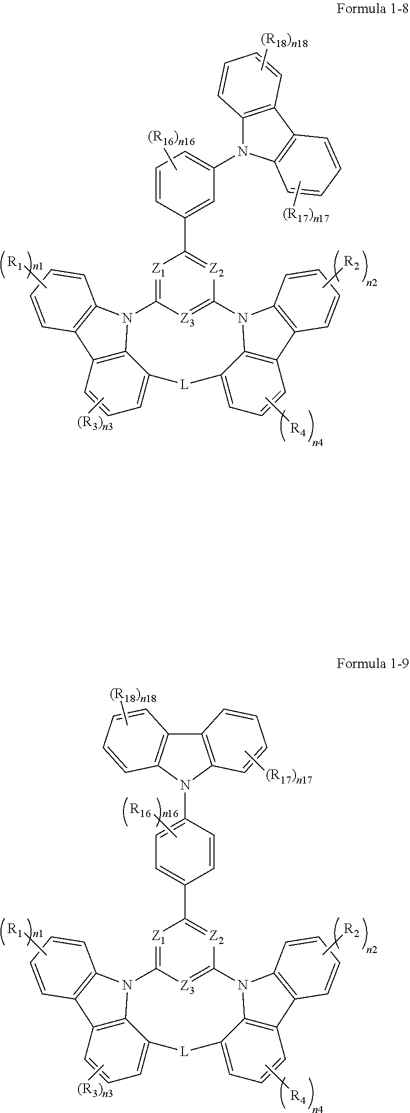

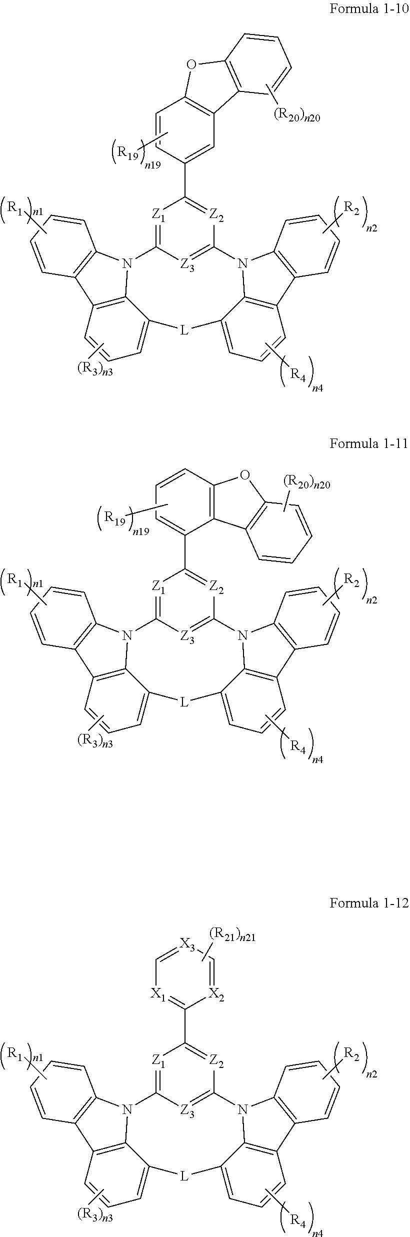

In Formulas 1-3 to 1-12, the same descriptions as in Formulas 1 may also be applied to Z₁ to Z₃, R₁ to R₄, n1 to n4, and L.

In Formulas 1-3 to 1-12, the same descriptions as in Formulas 3-1 to 3-5 above may also be applied to X₁ to X₃, R₁₁ to R₂₂, and n11 to n21.

In one or more embodiments, the hetero compound represented by Formula 1 may be represented by any one selected from among Formulas 1-13 to 1-16.

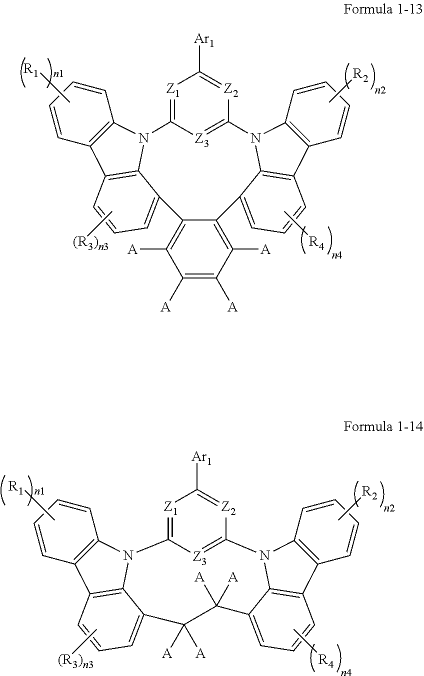

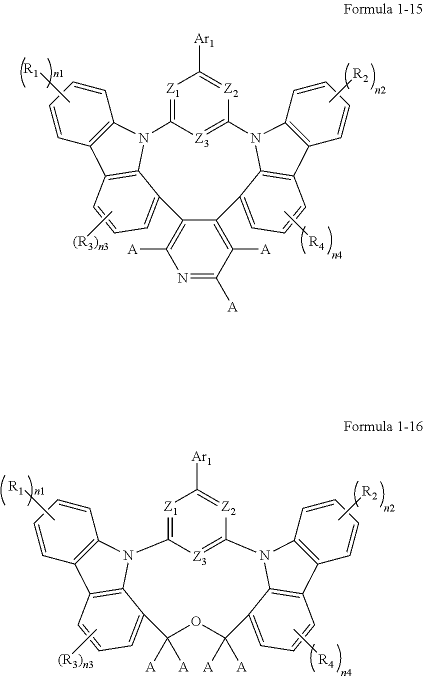

In Formulas 1-13 to 1-16, the same descriptions as in Formula 1 may also be applied to Z₁ to Z₃, R₁ to R₄, n1 to n4, and Ar₁.

In Formulas 1-13 to 1-16, the same descriptions as in Formula 2-5 to 2-8 may also be applied to A.

In one or more embodiments, the hetero compound of one or more embodiments represented by Formula 1 may include at least one deuterium as a substituent.

The hetero compound of one or more embodiments may be any one of compounds shown in Compound Group 1. A light emitting element ED of one or more embodiments may include, as a first compound, at least one hetero compound selected from among the compounds shown in Compound Group 1 in an emission layer EML.

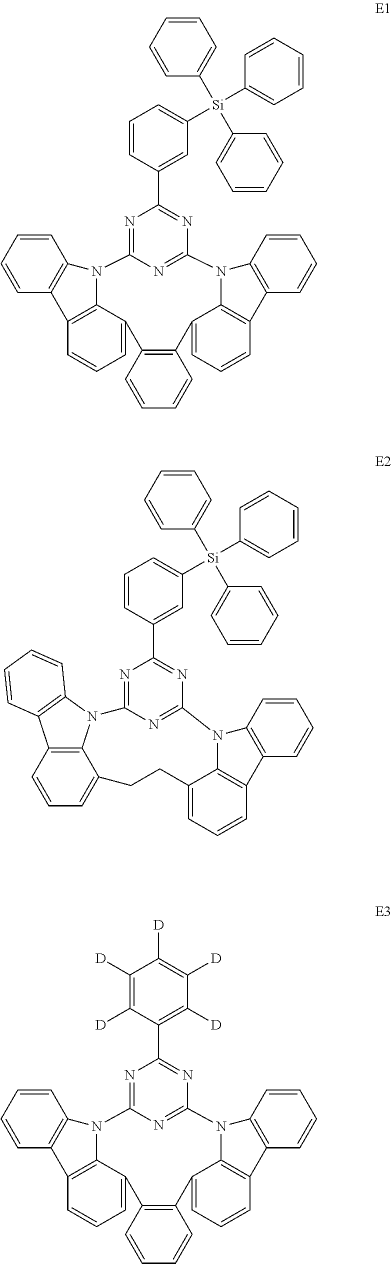 [SMILES: c1ccc([Si](c2ccccc2)(c2ccccc2)c2cccc(-c3nc4nc(n3)n3c5ccccc5c5cccc(c6ccccc6c6cccc7c8ccccc8n4c67)c53)c2)cc1 ; c1ccc([Si](c2ccccc2)(c2ccccc2)c2cccc(-c3nc4nc(n3)-n3c5ccccc5c5cccc(c53)CCc3cccc5c6ccccc6n-4c35)c2)cc1 ; [2H]c1c([2H])c([2H])c(-c2nc3nc(n2)n2c4ccccc4c4cccc(c5ccccc5c5cccc6c7ccccc7n3c56)c42)c([2H])c1[2H]]

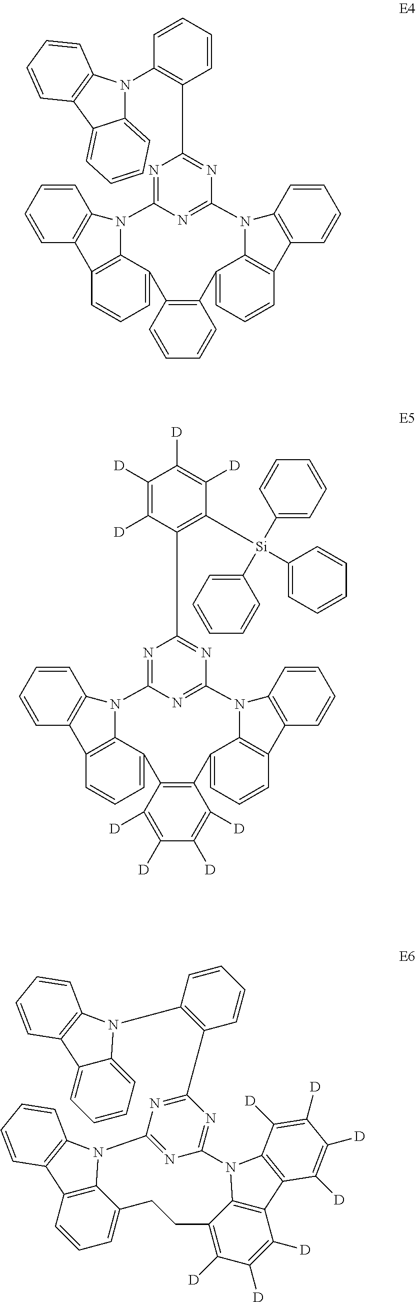 [SMILES: c1ccc(-n2c3ccccc3c3ccccc32)c(-c2nc3nc(n2)n2c4ccccc4c4cccc(c5ccccc5c5cccc6c7ccccc7n3c56)c42)c1 ; [2H]c1c([2H])c([2H])c([Si](c2ccccc2)(c2ccccc2)c2ccccc2)c(-c2nc3nc(n2)n2c4ccccc4c4cccc(c5c([2H])c([2H])c([2H])c([2H])c5c5cccc6c7ccccc7n3c65)c42)c1[2H] ; [2H]c1c([2H])c([2H])c2c(c1[2H])c1c([2H])c([2H])c([2H])c3c1n2-c1nc(-c2ccccc2-n2c4ccccc4c4ccccc42)nc(n1)-n1c2ccccc2c2cccc(c21)CC3]

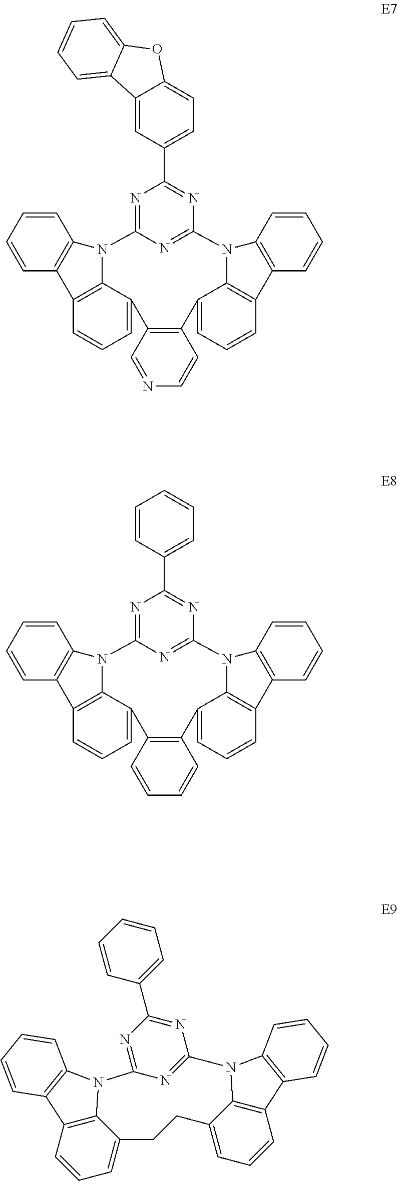 [SMILES: c1ccc2c(c1)oc1ccc(-c3nc4nc(n3)n3c5ccccc5c5cccc(c6cnccc6c6cccc7c8ccccc8n4c67)c53)cc12 ; c1ccc(-c2nc3nc(n2)n2c4ccccc4c4cccc(c5ccccc5c5cccc6c7ccccc7n3c56)c42)cc1 ; c1ccc(-c2nc3nc(n2)-n2c4ccccc4c4cccc(c42)CCc2cccc4c5ccccc5n-3c24)cc1]

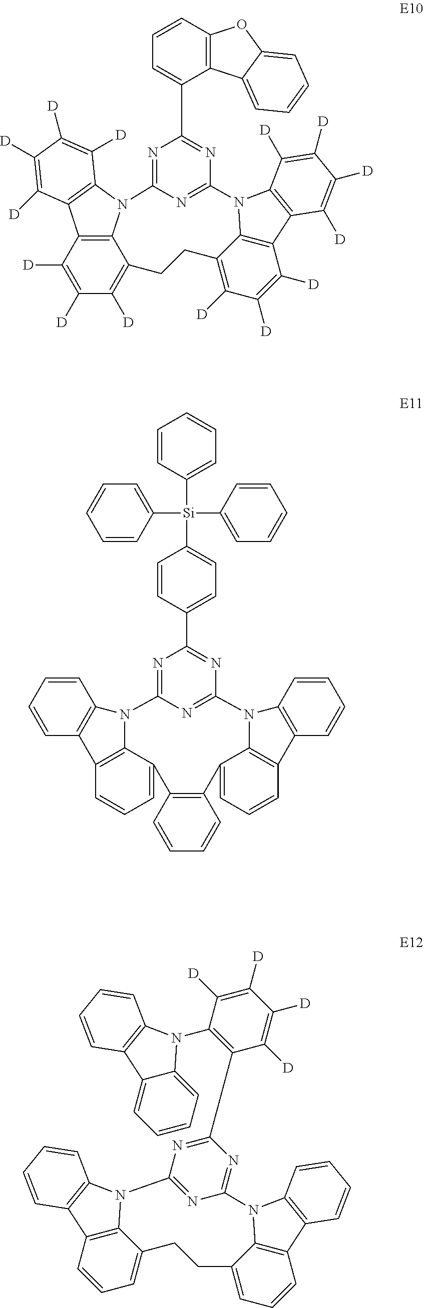 [SMILES: [2H]c1c([2H])c([2H])c2c(c1[2H])c1c([2H])c([2H])c([2H])c3c1n2-c1nc(-c2cccc4oc5ccccc5c24)nc(n1)-n1c2c([2H])c([2H])c([2H])c([2H])c2c2c([2H])c([2H])c([2H])c(c21)CC3 ; c1ccc([Si](c2ccccc2)(c2ccccc2)c2ccc(-c3nc4nc(n3)n3c5ccccc5c5cccc(c6ccccc6c6cccc7c8ccccc8n4c67)c53)cc2)cc1 ; [2H]c1c([2H])c([2H])c(-n2c3ccccc3c3ccccc32)c(-c2nc3nc(n2)-n2c4ccccc4c4cccc(c42)CCc2cccc4c5ccccc5n-3c24)c1[2H]]

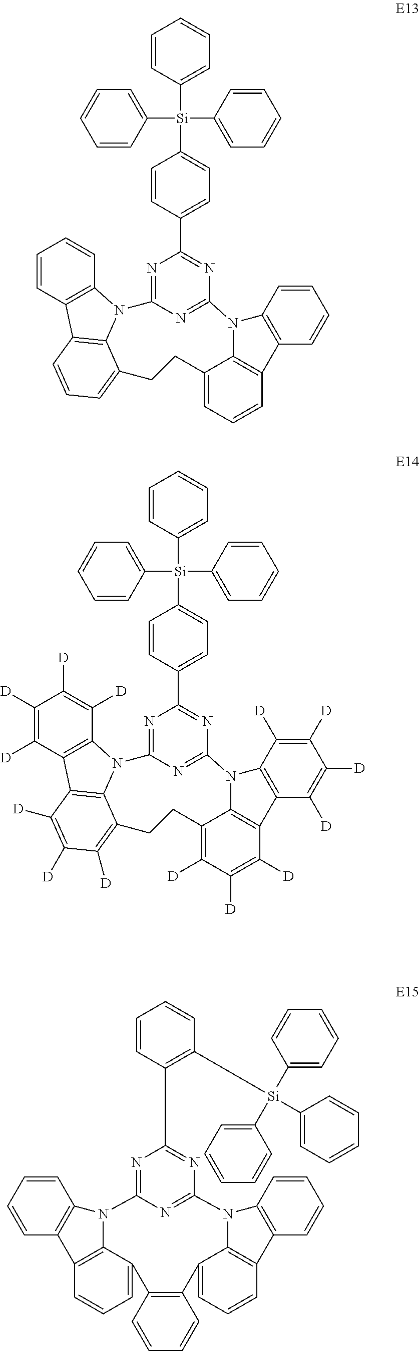 [SMILES: c1ccc([Si](c2ccccc2)(c2ccccc2)c2ccc(-c3nc4nc(n3)-n3c5ccccc5c5cccc(c53)CCc3cccc5c6ccccc6n-4c35)cc2)cc1 ; [2H]c1c([2H])c([2H])c2c(c1[2H])c1c([2H])c([2H])c([2H])c3c1n2-c1nc(-c2ccc([Si](c4ccccc4)(c4ccccc4)c4ccccc4)cc2)nc(n1)-n1c2c([2H])c([2H])c([2H])c([2H])c2c2c([2H])c([2H])c([2H])c(c21)CC3 ; c1ccc([Si](c2ccccc2)(c2ccccc2)c2ccccc2-c2nc3nc(n2)n2c4ccccc4c4cccc(c5ccccc5c5cccc6c7ccccc7n3c56)c42)cc1]

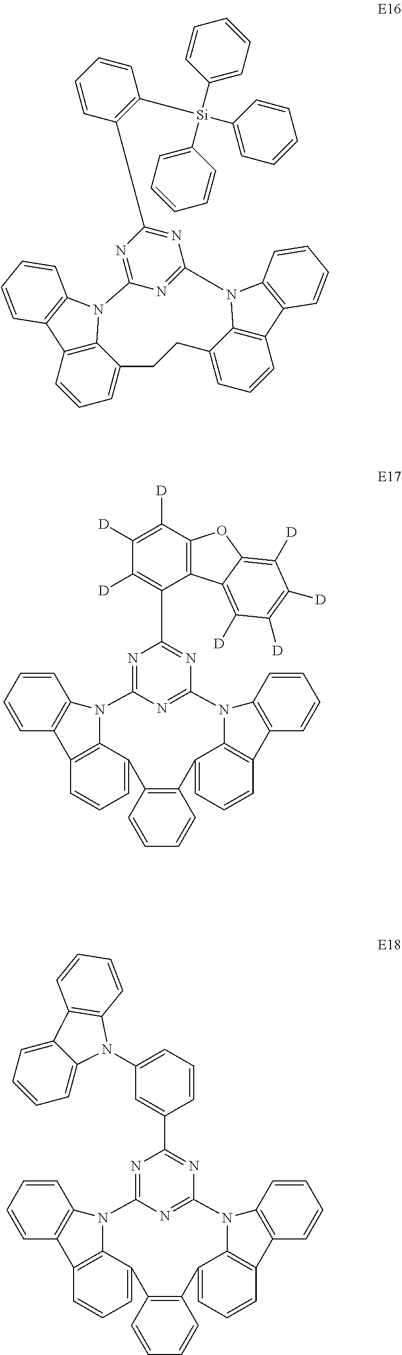 [SMILES: c1ccc([Si](c2ccccc2)(c2ccccc2)c2ccccc2-c2nc3nc(n2)-n2c4ccccc4c4cccc(c42)CCc2cccc4c5ccccc5n-3c24)cc1 ; [2H]c1c([2H])c([2H])c2c(oc3c([2H])c([2H])c([2H])c(-c4nc5nc(n4)n4c6ccccc6c6cccc(c7ccccc7c7cccc8c9ccccc9n5c78)c64)c32)c1[2H] ; c1cc(-c2nc3nc(n2)n2c4ccccc4c4cccc(c5ccccc5c5cccc6c7ccccc7n3c56)c42)cc(-n2c3ccccc3c3ccccc32)c1]

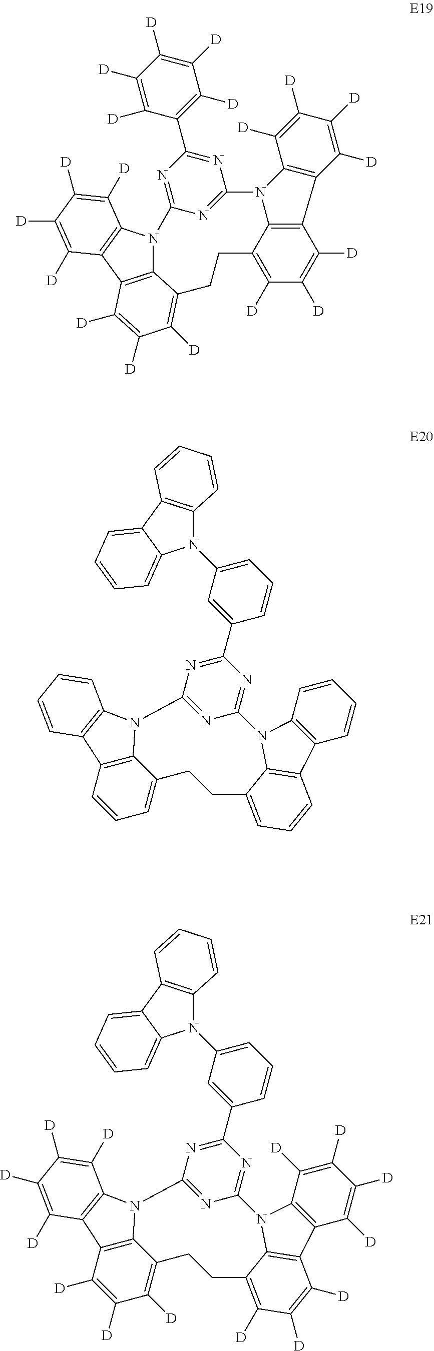 [SMILES: [2H]c1c([2H])c([2H])c(-c2nc3nc(n2)-n2c4c([2H])c([2H])c([2H])c([2H])c4c4c([2H])c([2H])c([2H])c(c42)CCc2c([2H])c([2H])c([2H])c4c5c([2H])c([2H])c([2H])c([2H])c5n-3c24)c([2H])c1[2H] ; c1cc(-c2nc3nc(n2)-n2c4ccccc4c4cccc(c42)CCc2cccc4c5ccccc5n-3c24)cc(-n2c3ccccc3c3ccccc32)c1 ; [2H]c1c([2H])c([2H])c2c(c1[2H])c1c([2H])c([2H])c([2H])c3c1n2-c1nc(-c2cccc(-n4c5ccccc5c5ccccc54)c2)nc(n1)-n1c2c([2H])c([2H])c([2H])c([2H])c2c2c([2H])c([2H])c([2H])c(c21)CC3]

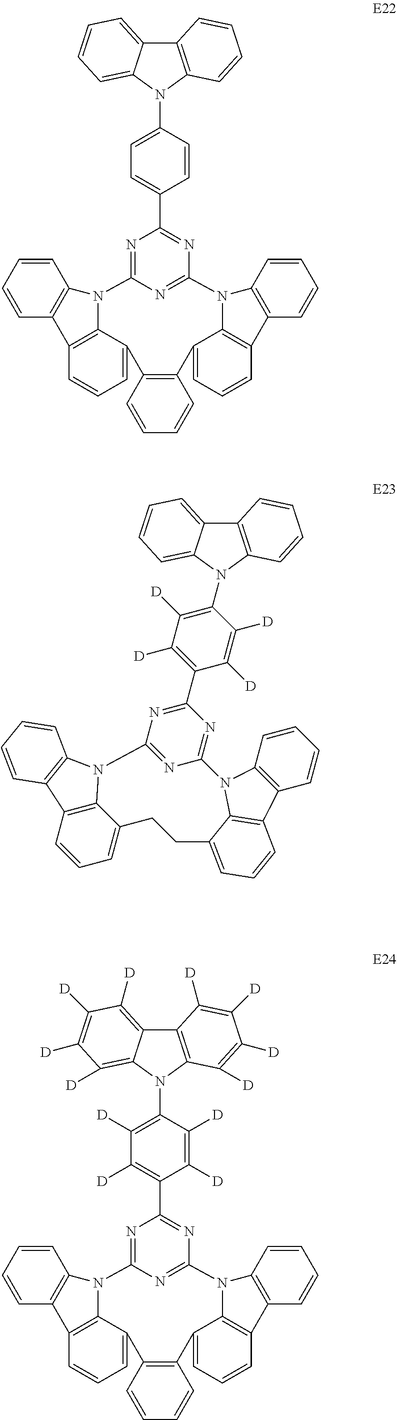 [SMILES: c1ccc2c(c1)c1cccc3c4ccccc4n(c4nc(-c5ccc(-n6c7ccccc7c7ccccc76)cc5)nc(n4)n4c5ccccc5c5cccc2c54)c13 ; [2H]c1c([2H])c(-n2c3ccccc3c3ccccc32)c([2H])c([2H])c1-c1nc2nc(n1)-n1c3ccccc3c3cccc(c31)CCc1cccc3c4ccccc4n-2c13 ; [2H]c1c([2H])c(-n2c3c([2H])c([2H])c([2H])c([2H])c3c3c([2H])c([2H])c([2H])c([2H])c32)c([2H])c([2H])c1-c1nc2nc(n1)n1c3ccccc3c3cccc(c4ccccc4c4cccc5c6ccccc6n2c45)c31]

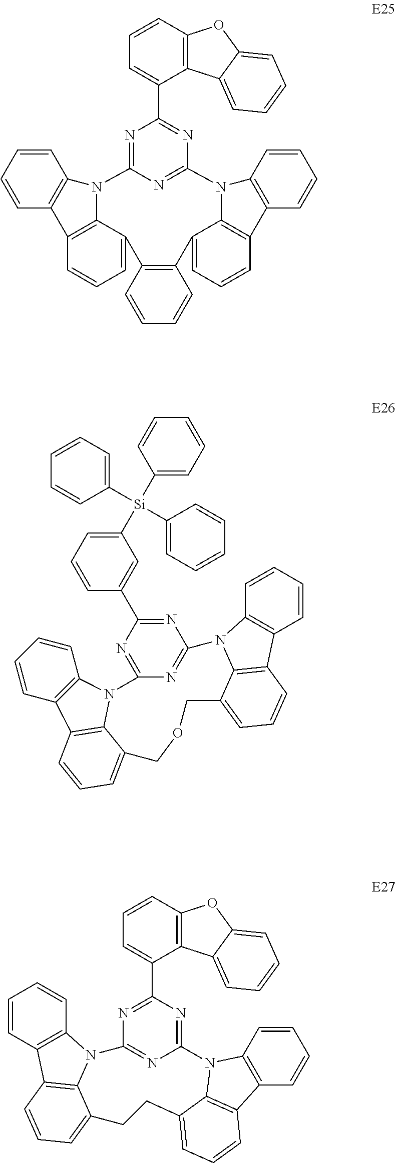 [SMILES: c1ccc2c(c1)oc1cccc(-c3nc4nc(n3)n3c5ccccc5c5cccc(c6ccccc6c6cccc7c8ccccc8n4c67)c53)c12 ; c1ccc([Si](c2ccccc2)(c2ccccc2)c2cccc(-c3nc4nc(n3)-n3c5ccccc5c5cccc(c53)COCc3cccc5c6ccccc6n-4c35)c2)cc1 ; c1ccc2c(c1)oc1cccc(-c3nc4nc(n3)-n3c5ccccc5c5cccc(c53)CCc3cccc5c6ccccc6n-4c35)c12]

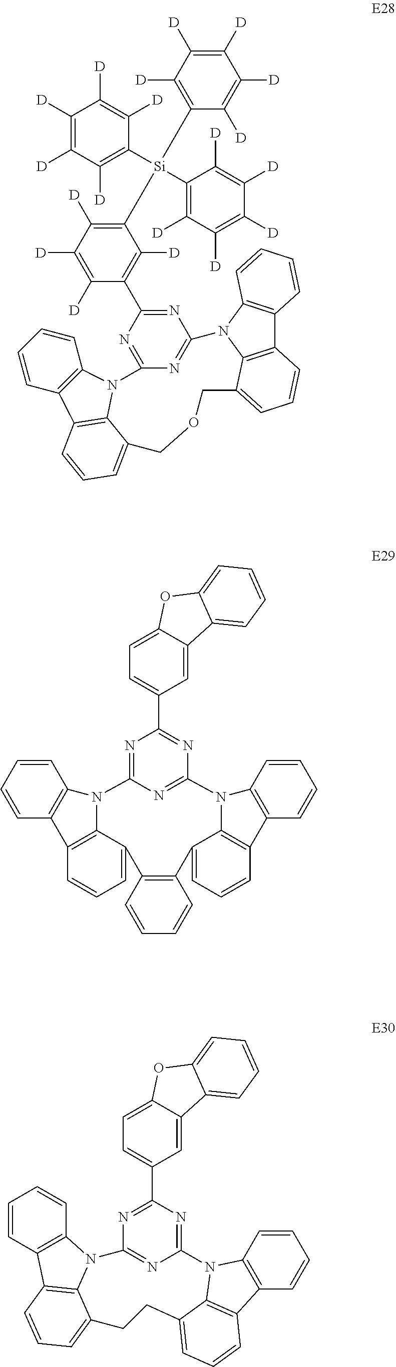 [SMILES: [2H]c1c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c(-c3nc4nc(n3)-n3c5ccccc5c5cccc(c53)COCc3cccc5c6ccccc6n-4c35)c2[2H])c([2H])c1[2H] ; c1ccc2c(c1)oc1ccc(-c3nc4nc(n3)n3c5ccccc5c5cccc(c6ccccc6c6cccc7c8ccccc8n4c67)c53)cc12 ; c1ccc2c(c1)oc1ccc(-c3nc4nc(n3)-n3c5ccccc5c5cccc(c53)CCc3cccc5c6ccccc6n-4c35)cc12]

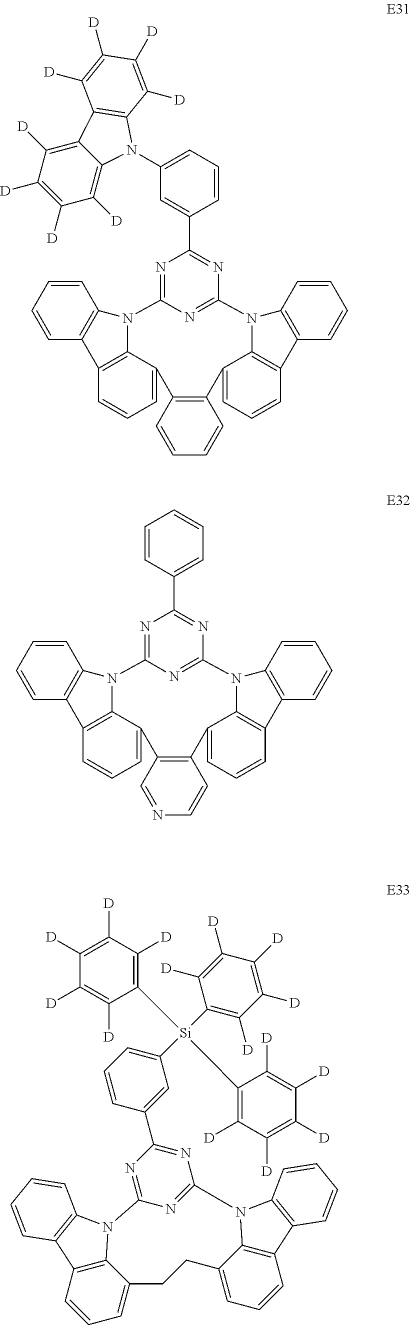 [SMILES: [2H]c1c([2H])c([2H])c2c(c1[2H])c1c([2H])c([2H])c([2H])c([2H])c1n2-c1cccc(-c2nc3nc(n2)n2c4ccccc4c4cccc(c5ccccc5c5cccc6c7ccccc7n3c56)c42)c1 ; c1ccc(-c2nc3nc(n2)n2c4ccccc4c4cccc(c5cnccc5c5cccc6c7ccccc7n3c56)c42)cc1 ; [2H]c1c([2H])c([2H])c([Si](c2cccc(-c3nc4nc(n3)-n3c5ccccc5c5cccc(c53)CCc3cccc5c6ccccc6n-4c35)c2)(c2c([2H])c([2H])c([2H])c([2H])c2[2H])c2c([2H])c([2H])c([2H])c([2H])c2[2H])c([2H])c1[2H]]

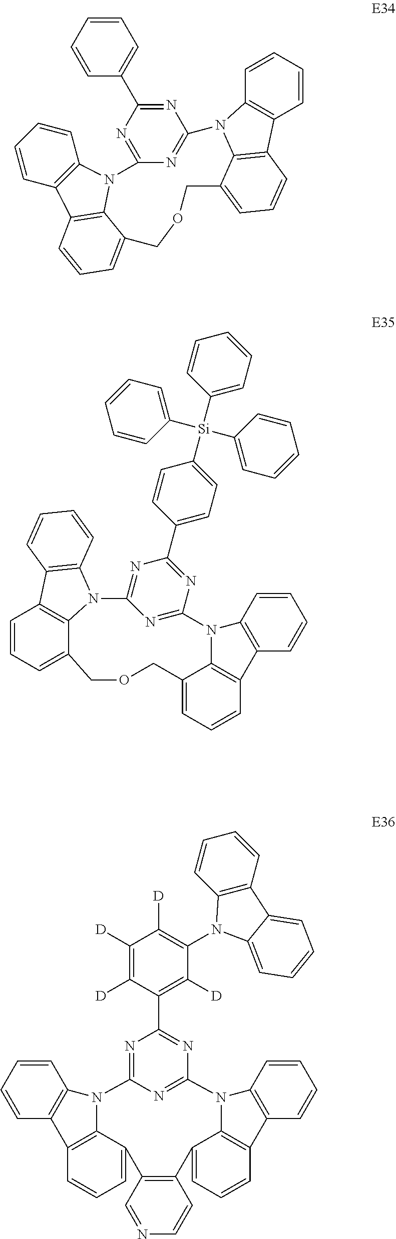 [SMILES: c1ccc(-c2nc3nc(n2)-n2c4ccccc4c4cccc(c42)COCc2cccc4c5ccccc5n-3c24)cc1 ; c1ccc([Si](c2ccccc2)(c2ccccc2)c2ccc(-c3nc4nc(n3)-n3c5ccccc5c5cccc(c53)COCc3cccc5c6ccccc6n-4c35)cc2)cc1 ; [2H]c1c([2H])c(-c2nc3nc(n2)n2c4ccccc4c4cccc(c5cnccc5c5cccc6c7ccccc7n3c56)c42)c([2H])c(-n2c3ccccc3c3ccccc32)c1[2H]]

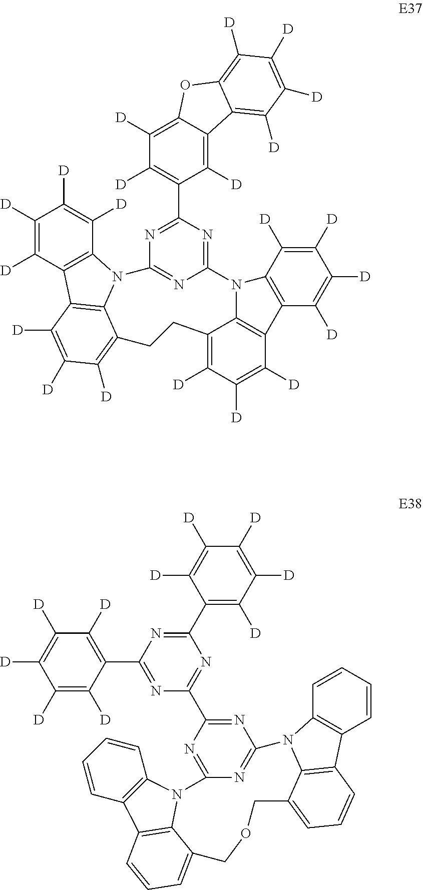 [SMILES: [2H]c1c([2H])c([2H])c2c(oc3c([2H])c([2H])c(-c4nc5nc(n4)-n4c6c([2H])c([2H])c([2H])c([2H])c6c6c([2H])c([2H])c([2H])c(c64)CCc4c([2H])c([2H])c([2H])c6c7c([2H])c([2H])c([2H])c([2H])c7n-5c46)c([2H])c32)c1[2H] ; [2H]c1c([2H])c([2H])c(-c2nc(-c3nc4nc(n3)-n3c5ccccc5c5cccc(c53)COCc3cccc5c6ccccc6n-4c35)nc(-c3c([2H])c([2H])c([2H])c([2H])c3[2H])n2)c([2H])c1[2H]]

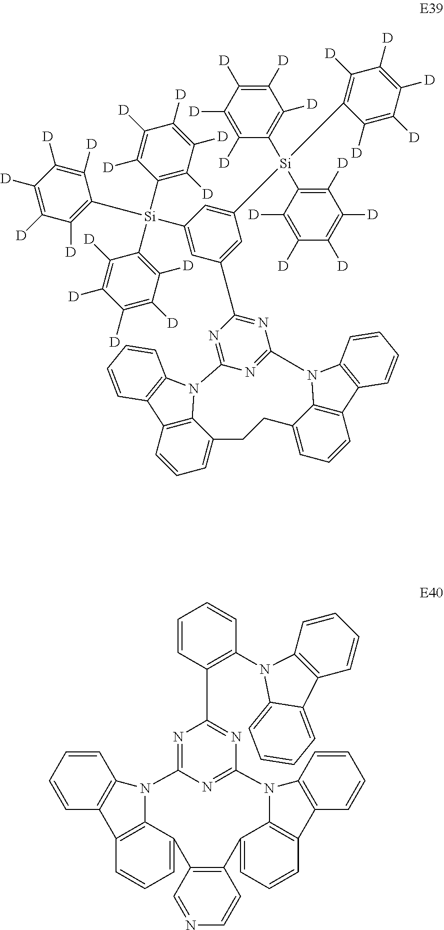 [SMILES: [2H]c1c([2H])c([2H])c([Si](c2cc(-c3nc4nc(n3)-n3c5ccccc5c5cccc(c53)CCc3cccc5c6ccccc6n-4c35)cc([Si](c3c([2H])c([2H])c([2H])c([2H])c3[2H])(c3c([2H])c([2H])c([2H])c([2H])c3[2H])c3c([2H])c([2H])c([2H])c([2H])c3[2H])c2)(c2c([2H])c([2H])c([2H])c([2H])c2[2H])c2c([2H])c([2H])c([2H])c([2H])c2[2H])c([2H])c1[2H] ; c1ccc(-n2c3ccccc3c3ccccc32)c(-c2nc3nc(n2)n2c4ccccc4c4cccc(c5cnccc5c5cccc6c7ccccc7n3c56)c42)c1]

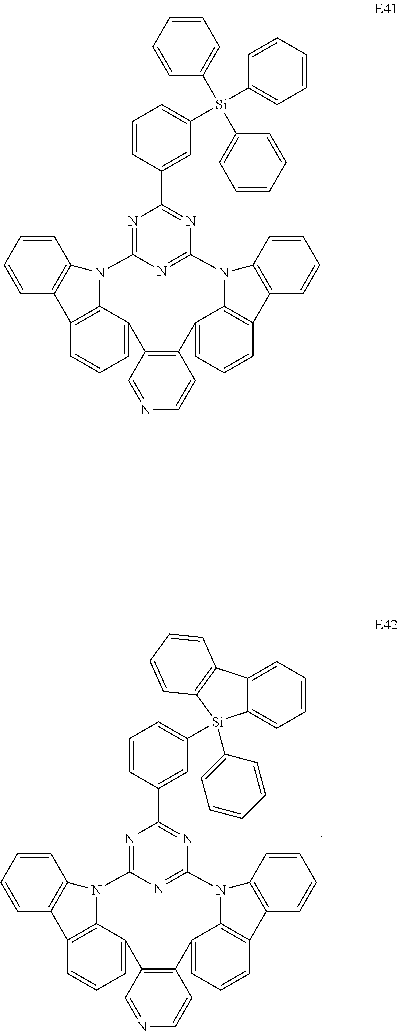 [SMILES: c1ccc([Si](c2ccccc2)(c2ccccc2)c2cccc(-c3nc4nc(n3)n3c5ccccc5c5cccc(c6cnccc6c6cccc7c8ccccc8n4c67)c53)c2)cc1 ; c1ccc([Si]2(c3cccc(-c4nc5nc(n4)n4c6ccccc6c6cccc(c7cnccc7c7cccc8c9ccccc9n5c78)c64)c3)c3ccccc3-c3ccccc32)cc1]

In the example compounds presented in Compound Group 1, “D” indicates deuterium.

The hetero compound represented by Formula 1 according to one or more embodiments has a structure in which a first substituent is introduced, and may thus achieve high light emitting efficiency and long lifespan.

The hetero compound of one or more embodiments represented by Formula 1 may include a structure in which a nitrogen-containing heterocycle and two carbazole moieties connected to the nitrogen-containing heterocycle are included, the two carbazole moieties are connected through a linker. In one or more embodiments, the linker connecting the two carbazole moieties includes a divalent oxy group, a divalent thio group, a divalent silyl group, an alkylene group, an arylene group, or a heteroarylene group. In one or more embodiments, the linker connecting the two carbazole moieties has a structure connected to each of the carbon atoms positioned ortho with respect to the carbon atom connected to a nitrogen atom among the carbon atoms constituting the carbazole moiety.

The hetero compound of one or more embodiments has a structure in which a hexagonal (e.g., a six-membered) heterocycle containing 1 to 3 nitrogen atoms as ring-forming atoms is a core structure and two carbazole moieties are connected to the hexagonal heterocycle, and thus has a high triplet energy level, and may thus be utilized as a host material for deep blue phosphorescence or thermally activated delayed fluorescence. In one or more embodiments, the hetero compound of one or more embodiments may include a linker structure connecting two carbazole moieties, thereby making a connection structure of the nitrogen-containing heterocycle and the two carbazole moieties rigid to prevent or reduce vibronic motion of compound molecules in an excited state, and may thus have a high triplet energy level. Accordingly, when the hetero compound of one or more embodiments is applied as a host material for a phosphorescent light emitting element or thermally activated delayed fluorescence, the light emitting element has improved light emitting efficiency and lifespan.

The hetero compound of one or more embodiments represented by Formula 1 may be a phosphorescent host material, or a thermally activated delayed fluorescence (TADF) host material. In one or more embodiments, the hetero compound of one or more embodiments represented by Formula 1 may be a thermally activated delayed fluorescence host having a difference A EST between a lowest triplet excitation energy level (T1 level) and a lowest singlet excitation energy level (S1 level) of 0.6 eV or less.

In the light emitting element ED of one or more embodiments, the emission layer EML may be to emit phosphorescence or delayed fluorescence. For example, the emission layer EML may be to emit phosphorescence or thermally activated delayed fluorescence.

The hetero compound of one or more embodiments may be included in the emission layer EML. The hetero compound of one or more embodiments may be included in the emission layer EML as a host material. The hetero compound of one or more embodiments may be utilized as a host material for phosphorescence or as a host material for thermally activated delayed fluorescence. For example, in the light emitting element ED of one or more embodiments, the emission layer EML may include at least one of the hetero compounds shown in Compound Group 1 as a host material for phosphorescence or as a host material for thermally activated delayed fluorescence. However, the utilization of the hetero compound of one or more embodiments is not limited thereto.

A light-emitting element including the hetero compound of one or more embodiments represented by Formula 1 may include an emission layer EML having a central light emitting wavelength in a wavelength of about 430 nm to about 490 nm. For example, the hetero compound of one or more embodiments represented by Formula 1 may be a blue thermally activated delayed fluorescent host or a blue phosphorescent host. However, embodiments of the present disclosure are not limited thereto, for example, when the hetero compound of one or more embodiments is utilized as a light emitting host material, the first compound may be utilized as a host material emitting light in one or more suitable wavelength ranges, such as a red light emitting host and/or a green light emitting host.

In one or more embodiments, the emission layer EML may include a plurality of compounds. The emission layer EML of one or more embodiments may include the hetero compound represented by Formula 1, that is, the first compound, and may further include at least one of a second compound represented by Formula HT-1 or a third compound represented by Formula D-1.

In one or more embodiments, the emission layer EML may include the first compound represented by Formula 1 and may further include a second compound represented by Formula HT-1.

In one or more embodiments, the emission layer EML may include a second compound represented by Formula HT-1. In some embodiments, the second compound may be utilized as a hole transporting host material of the emission layer EML.

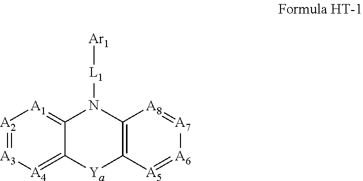

In Formula HT-1, A₁ to A₈ may each independently be N or CR₅₁. For example, in some embodiments, A₁ to A₈ may all be CR₅₁. In some embodiments, any one selected from among A₁ to A₈ may be N, and the others may be CR₅₁.

In Formula HT-1, L₁ may be a direct linkage, a substituted or unsubstituted arylene group having 6 to 30 ring-forming carbon atoms, or a substituted or unsubstituted heteroarylene group having 2 to 30 ring-forming carbon atoms. For example, in some embodiments, L₁ may be a direct linkage, a substituted or unsubstituted phenylene group, a substituted or unsubstituted divalent biphenyl group, a substituted or unsubstituted divalent carbazole group, and/or the like, but embodiments of the present disclosure are not limited thereto.

In Formula HT-1, Yₐ may be a direct linkage, CR₅₂R₅₃, or SiR₅₄R₅₅. For example, two benzene rings connected to nitrogen atom of Formula HT-1 may be connected through a direct linkage,

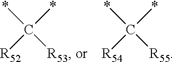

In Formula HT-1, when Yₐ is a direct linkage, a compound represented by Formula HT-1 may include a carbazole moiety.

In Formula HT-1, Ar₁ is a substituted or unsubstituted aryl group having 6 to 30 ring-forming carbon atoms, or a substituted or unsubstituted heteroaryl group having 2 to 30 ring-forming carbon atoms. For example, in some embodiments, Ar may be a substituted or unsubstituted carbazole group, a substituted or unsubstituted dibenzofuran group, a substituted or unsubstituted dibenzothiophene group, or a substituted or unsubstituted biphenyl group, but embodiments of the present disclosure are not limited thereto.

In Formula HT-1, R₅₁ to R₅₅ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 ring-forming carbon atoms, a substituted or unsubstituted aryl group having 6 to 60 ring-forming carbon atoms, or a substituted or unsubstituted heteroaryl group having 2 to 60 ring-forming carbon atoms. In some embodiments, one or more selected from R₅₁ to R₅₅ may each independently be bonded to an adjacent group to form a ring. For example, in some embodiments, R₅₁ to R₅₅ may each independently be hydrogen or deuterium. In some embodiments, R₅₁ to R₅₅ may each independently be an unsubstituted methyl group or an unsubstituted phenyl group.

In one or more embodiments, the second compound represented by Formula HT-1 may be represented by any one selected from among compounds shown in Compound Group 2. The emission layer EML may include at least one selected from among the compounds shown in Compound Group 2, as a hole transporting host material. In some embodiments, a second compound represented by Formula HT-1 may be included in the hole transport region HTR.

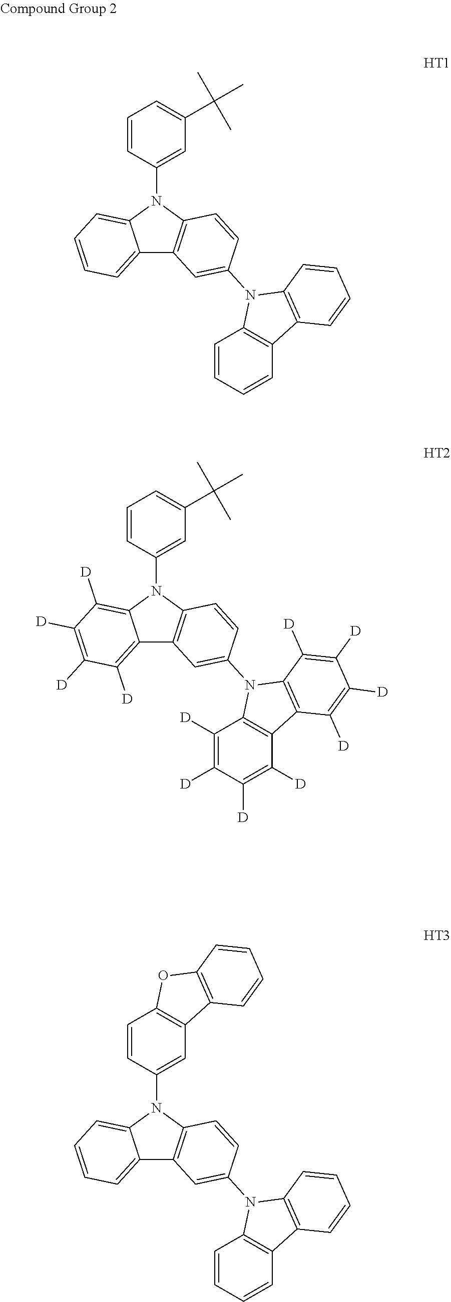 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

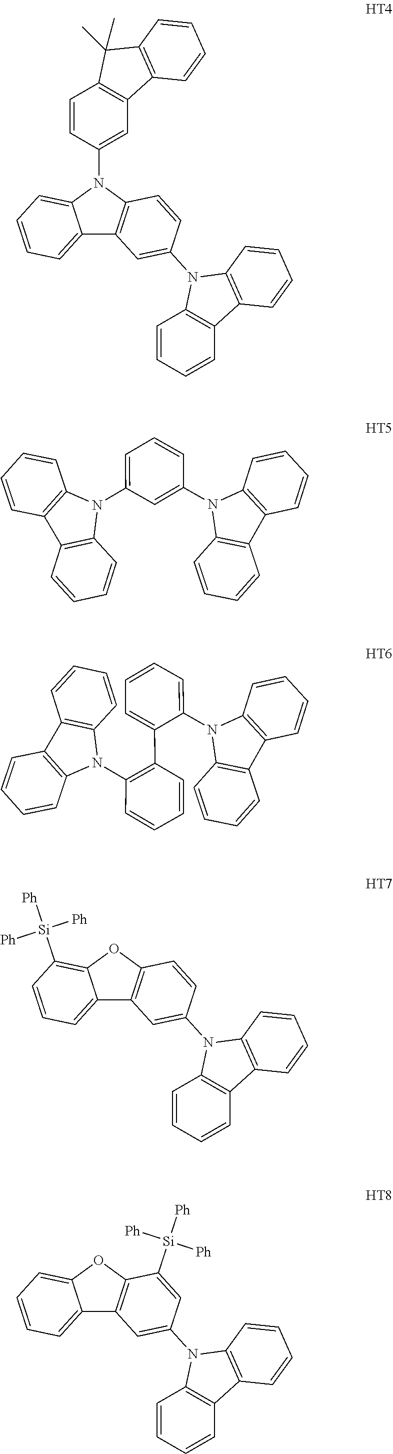 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

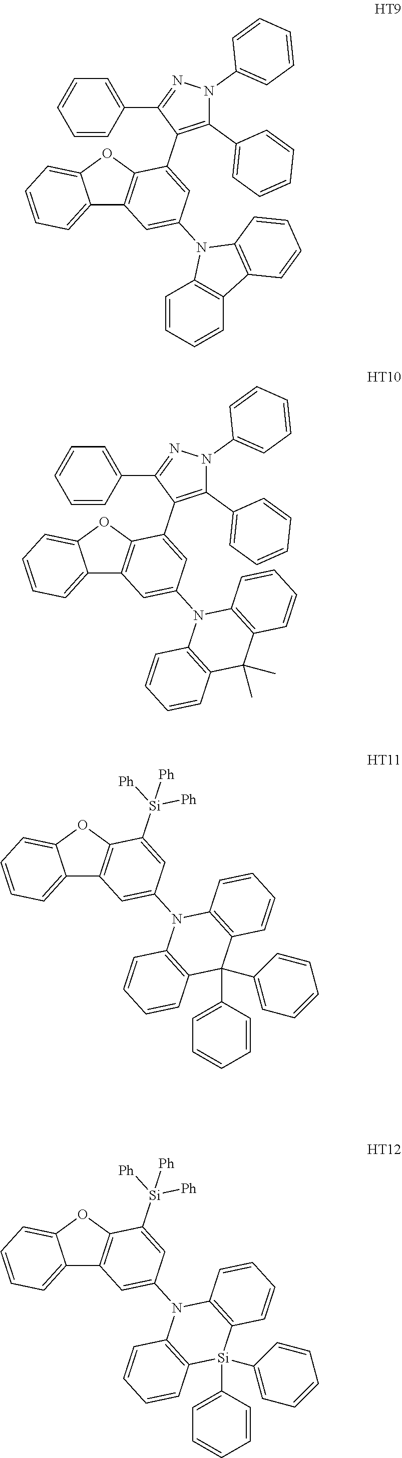 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

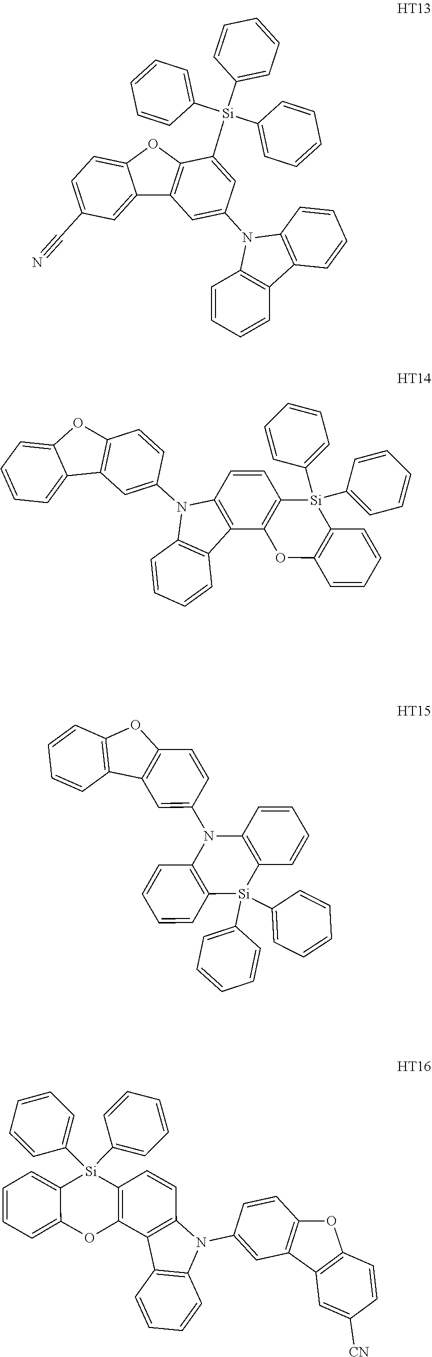 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1]

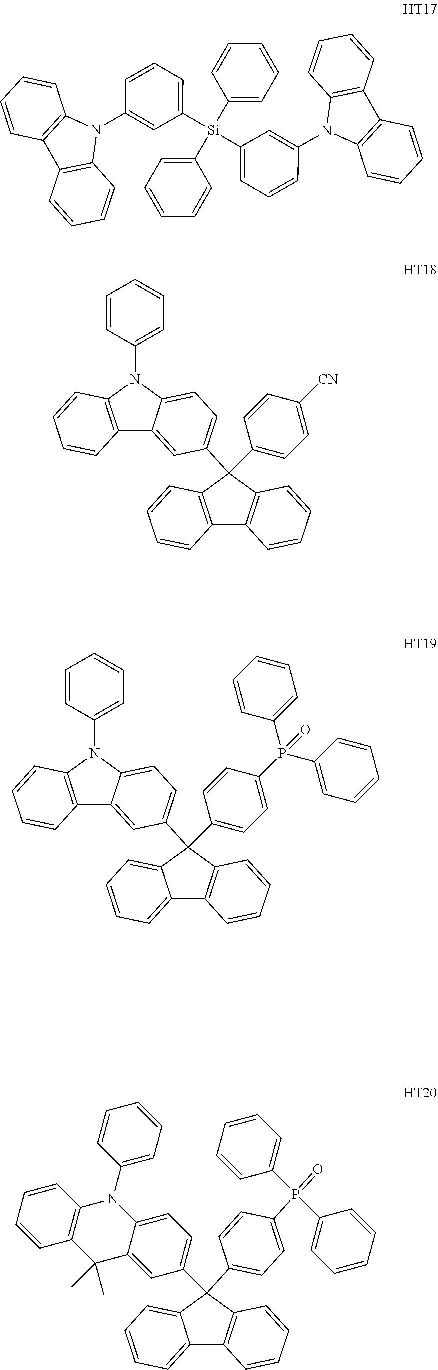 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

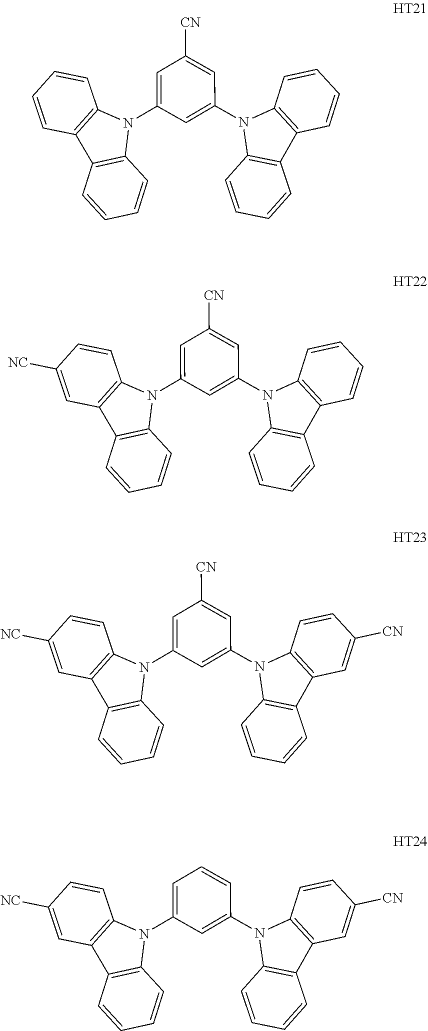 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)c1]

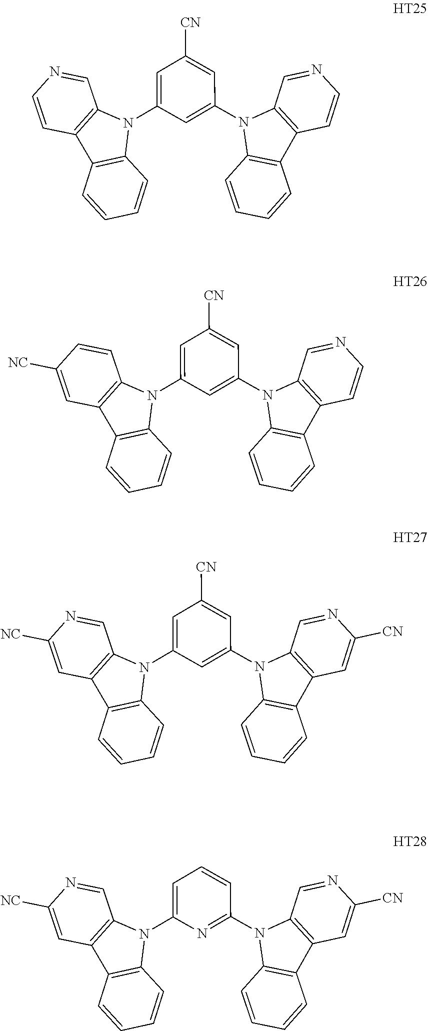 [SMILES: N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1]

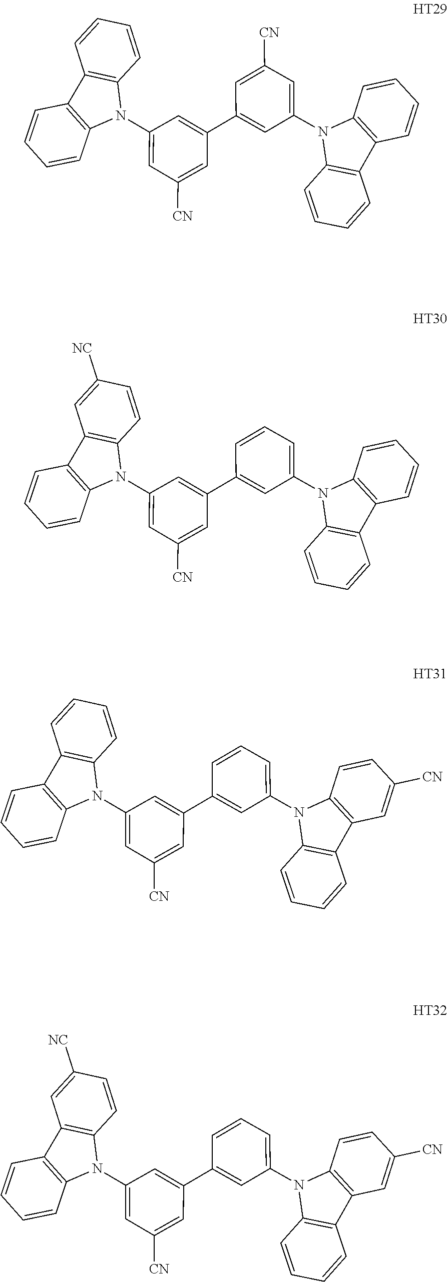 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1]

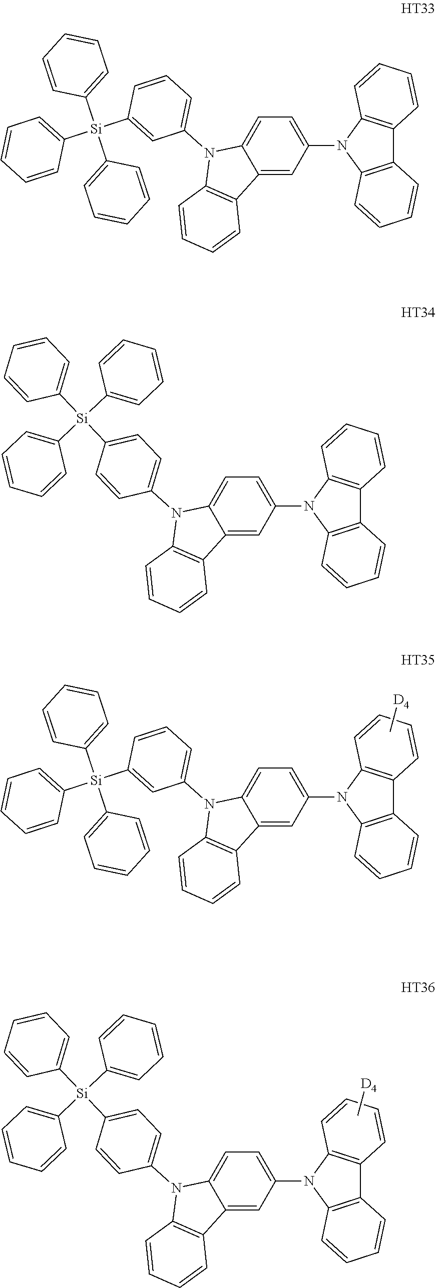

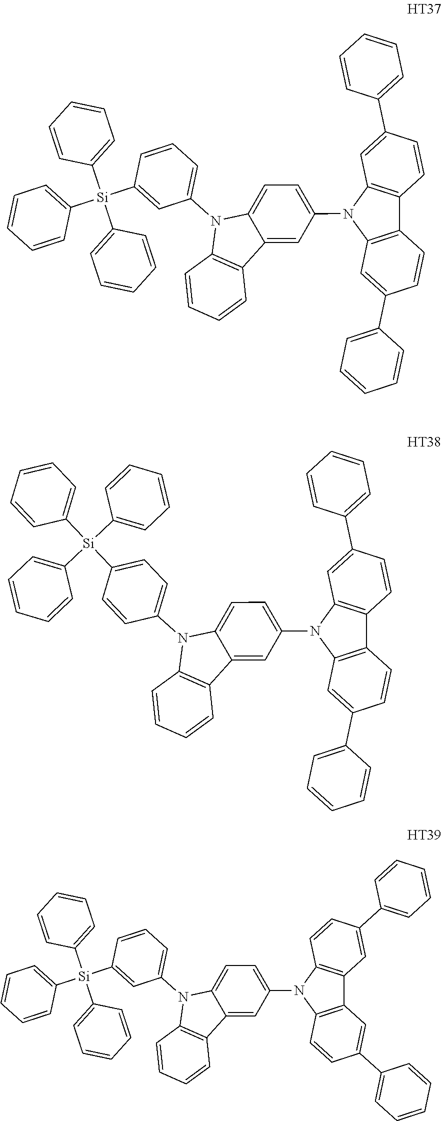 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1 ; c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1]

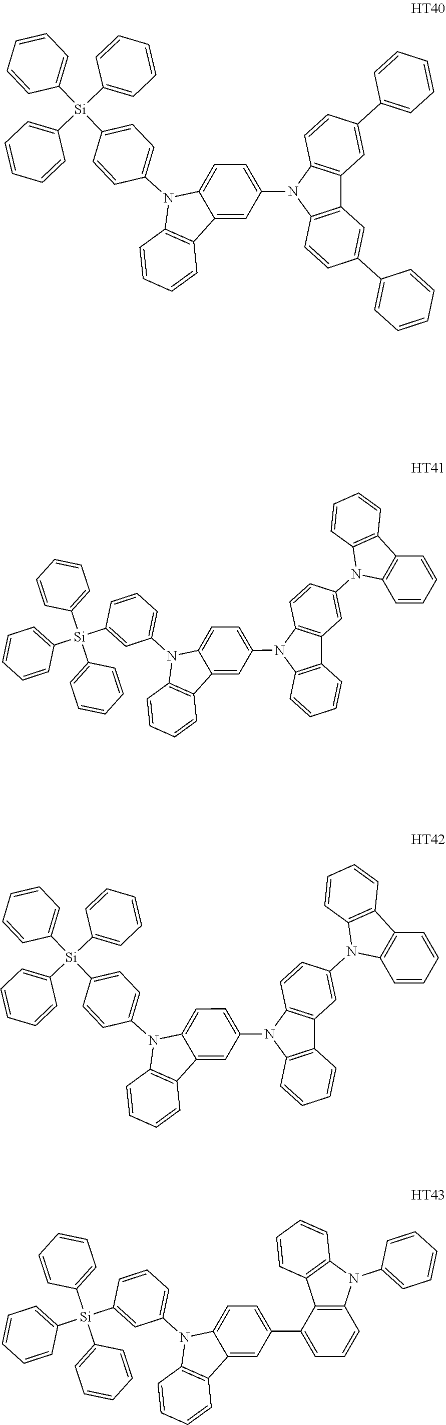 [SMILES: c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1 ; c1ccc(-n2c3ccccc3c3c(-c4ccc5c(c4)c4ccccc4n5-c4cccc([Si](c5ccccc5)(c5ccccc5)c5ccccc5)c4)cccc32)cc1]

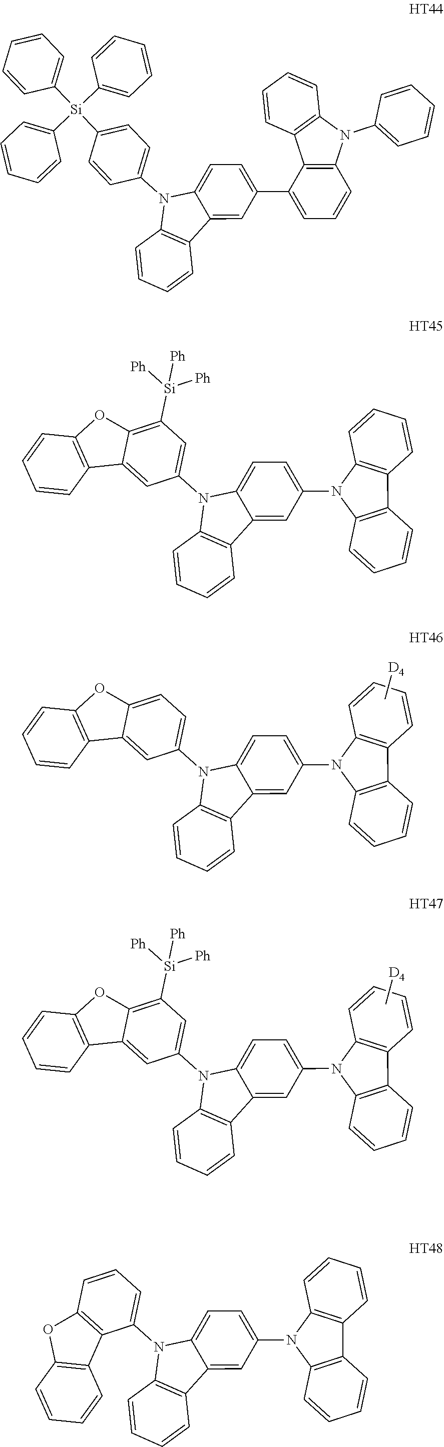

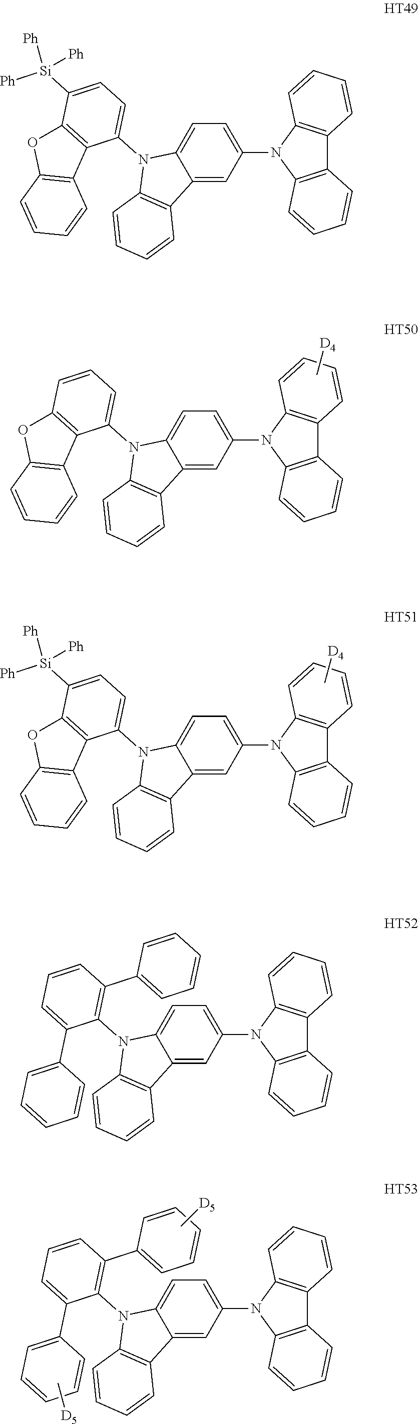

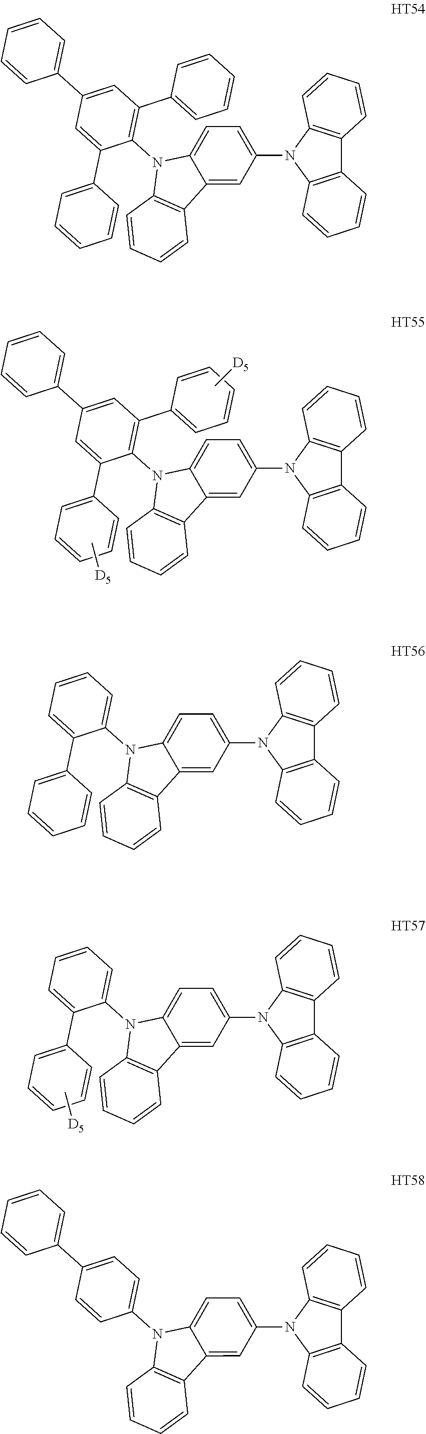

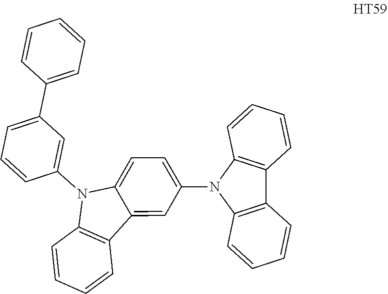 [SMILES: c1ccc(-c2cccc(-n3c4ccccc4c4cc(-n5c6ccccc6c6ccccc65)ccc43)c2)cc1]

In the example compounds presented in Compound Group 2, “D” may indicate deuterium, and “Ph” may indicate a substituted or unsubstituted phenyl group. For example, in the example compounds presented in Compound Group 2, “Ph” may be an unsubstituted phenyl group.

In one or more embodiments, the emission layer EML may include the first compound and the second compound, and the first compound and the second compound may form an exciplex. The emission layer EML may include the first compound represented by Formula 1 as an electron transporting host, and the second compound represented by Formula HT-1 as a hole transporting host, and may have an exciplex formed therein by the hole transporting host and the electron transporting host. In these embodiments, the triplet energy of the exciplex formed by the hole transporting host and the electron transporting host corresponds to a difference between Lowest Unoccupied Molecular Orbital (LUMO) energy level of the electron transporting host and Highest Occupied Molecular Orbital (HOMO) energy level of the hole transporting host.

For example, the triplet energy level (T1) of the exciplex formed by the hole transporting host and the electron transporting host may have an absolute value of about 2.4 eV to about 3.0 eV. In one or more embodiments, the triplet energy of the exciplex may have a value smaller than the energy gap of each host material. The exciplex may have a triplet energy of 3.0 eV or less, which is an energy gap between the hole transporting host and the electron transporting host.

In one or more embodiments, the emission layer EML may include a third compound in addition to the first compound and the second compound. The third compound may be utilized as a phosphorescent dopant of the emission layer EML.

For example, in one or more embodiments, the emission layer EML may include, as the third compound, an organometallic complex containing platinum (Pt) as a central metal atom and ligands bound to the central metal atom. In the light emitting element ED of one or more embodiments, the emission layer EML may include a compound represented by Formula D-1 as the third compound.

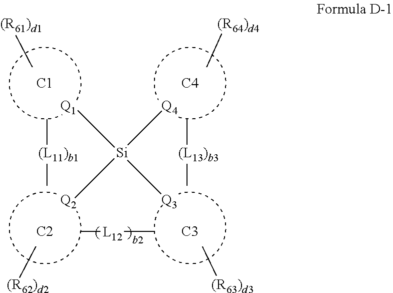

In Formula D-1, Q₁ to Q₄ may each independently be C or N.

In Formula D-1, C1 to C4 may each independently be a substituted or unsubstituted hydrocarbon ring having ring-forming 5 to 30 carbon atoms, or a substituted or unsubstituted heterocycle having 2 to 30 ring-forming carbon atoms.

In Formula D-1, L₁₁ to L₁₃ may each independently be a direct linkage,

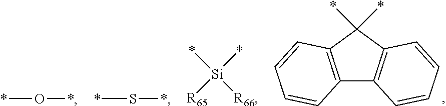

a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, In L₁₁ to L₁₃, “ [symbol] ” indicates a position connected to C1 to C4.

In Formula D-1, b1 to b3 may each independently be 0 or 1. When b1 is 0, C1 and C2 may not be connected. When b2 is 0, C2 and C3 may not be connected. When b3 is 0, C3 and C4 may not be connected.

In Formula D-1, R₆₁ to R₆₆ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 ring-forming carbon atoms, a substituted or unsubstituted aryl group having 6 to 60 ring-forming carbon atoms, or a substituted or unsubstituted heteroaryl group having 2 to 60 ring-forming carbon atoms. In some embodiments, one or more selected from among R₆₁ to R₆₆ may each independently be bonded to an adjacent group to form a ring. In some embodiments, R₆₁ to R₆₆ may each independently be a substituted or unsubstituted methyl group, or a substituted or unsubstituted t-butyl group.

In Formula D-1, d1 to d4 may each independently be an integer of 0 to 4. In Formula D-1, when d1 to d4 are each 0, the fourth compound may not be substituted with R₆₁ to R₆₄. When d1 to d4 are each 4 and R₆₁ to R₆₄ are each hydrogen, the embodiments may be the same as when d1 to d4 are each 0. When d1 to d4 are each an integer of 2 or greater, R₆₁ to R₆₄ provided in plurality may each be the same, or at least one of the plurality of R₆₁ to R₆₄ may be different.

In Formula D-1, C1 to C4 may each independently be a substituted or unsubstituted hydrocarbon ring or a substituted or unsubstituted heterocycle, which is represented by any one selected from among C-1 to C-4.

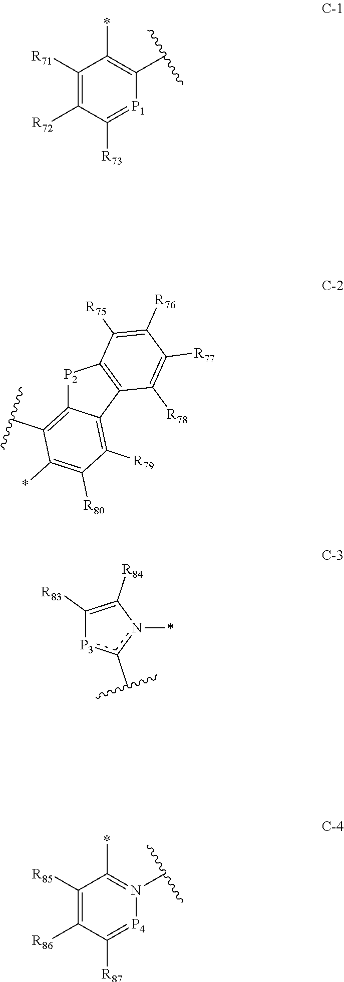

In C-1 to C-4, P₁- may be c [symbol] or CR₇₄, P₂ may be N [symbol] or NR₈₁, P₃ may be N [symbol] or NR₈₂, and P₄ may be c [symbol] or CR₈₈. R₇₁ to R₈₈ may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

In C-1 to C-4, “ [symbol] ” indicates a portion connected to the central metal atom, Pt, and “ [symbol] ” indicates a portion connected to neighboring ring groups (C1 to C4) or linkers (L₁₁ to L₁₃).

In one or more embodiments, the emission layer EML may include the first compound, the second compound, and the third compound. In the emission layer EML, the first compound and the second compound form an exciplex, and energy may be transferred from the exciplex to the third compound to emit light.

In one more embodiments, the third compound represented by Formula D-1 may be at least one selected from among compounds shown in Compound Group 3.

The emission layer EML may include at least one selected from among the compounds shown in Compound Group 3 as a phosphorescent dopant material.

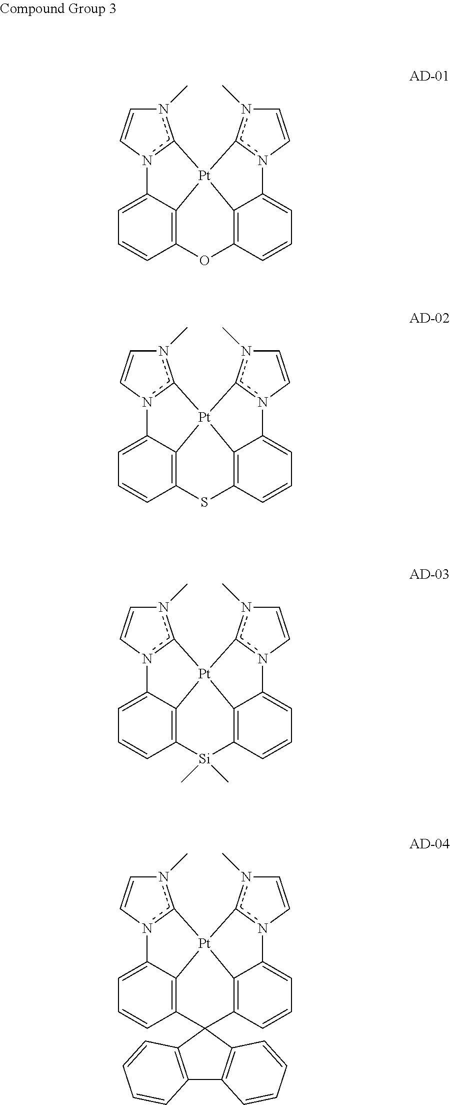

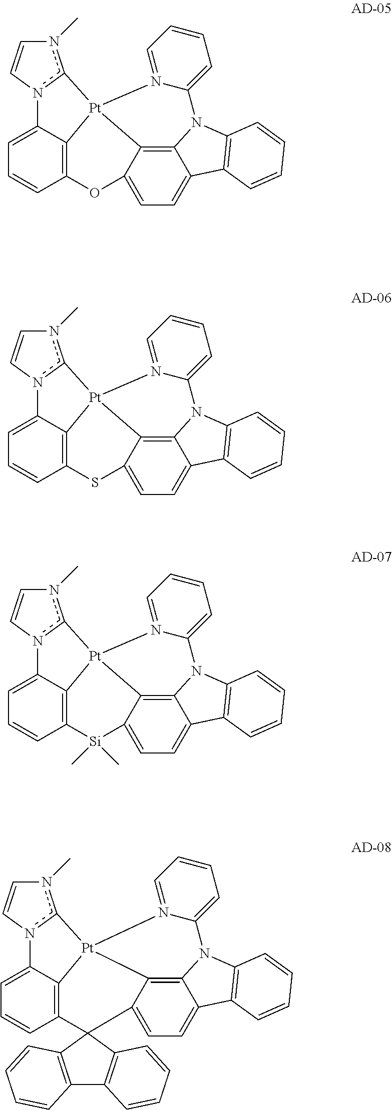

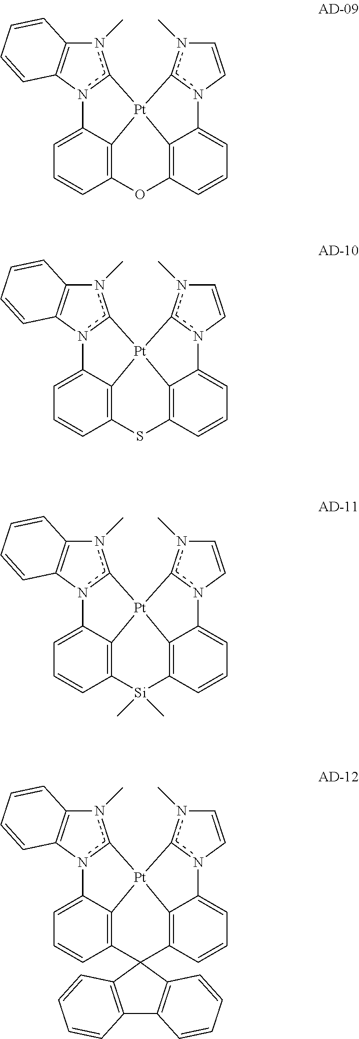

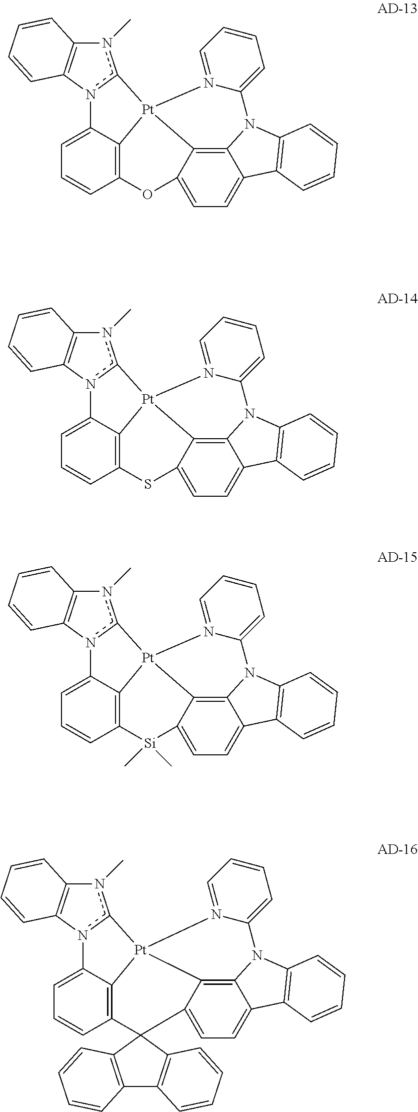

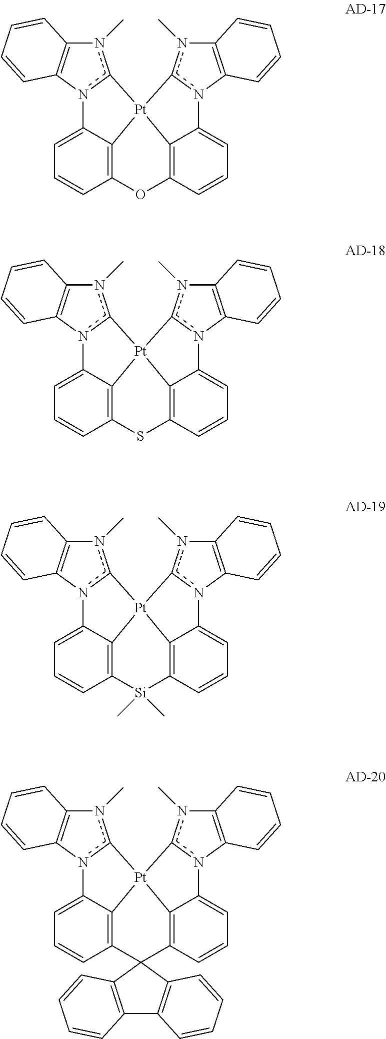

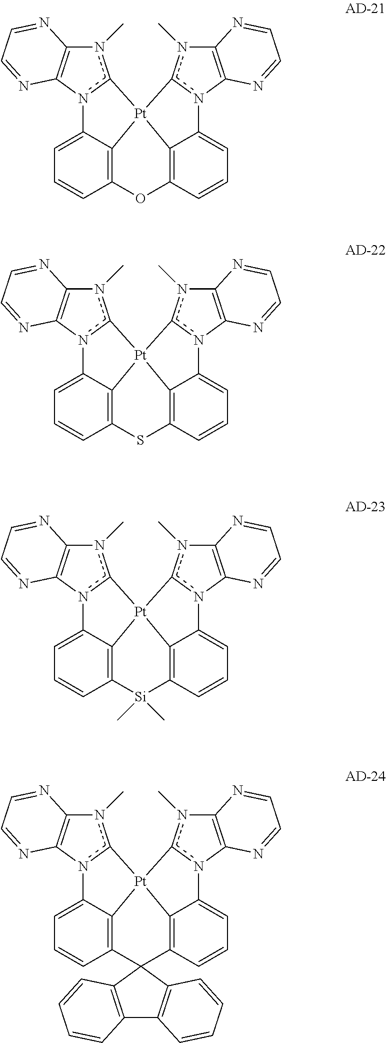

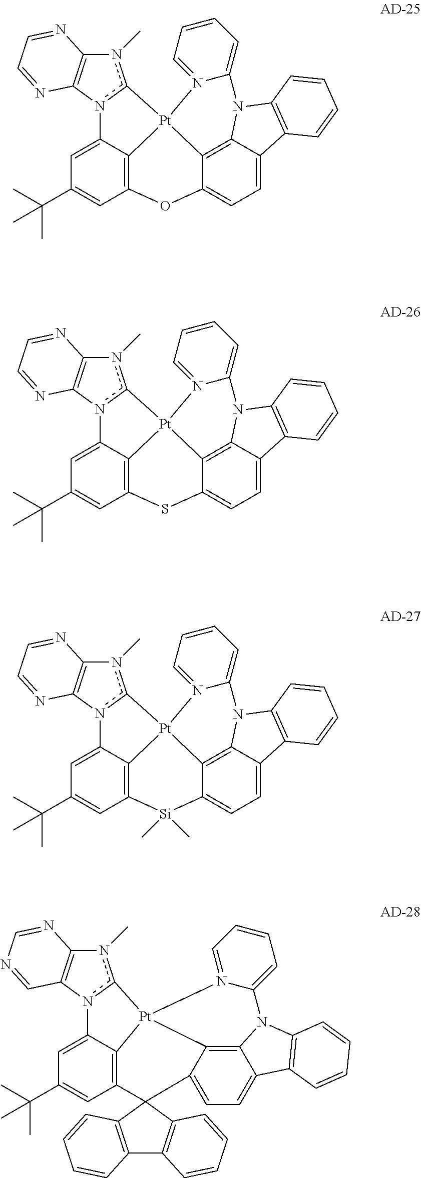

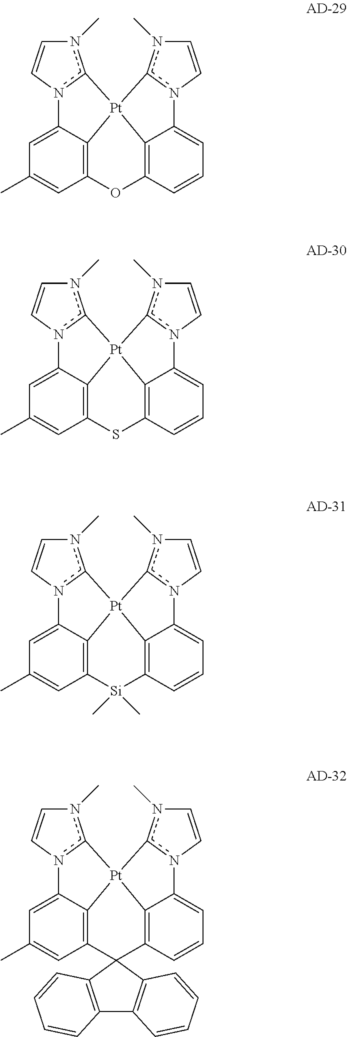

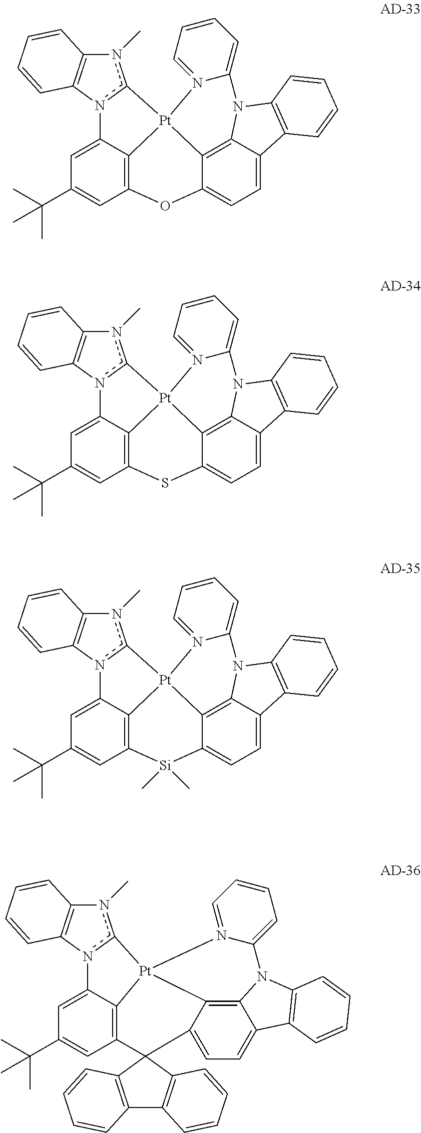

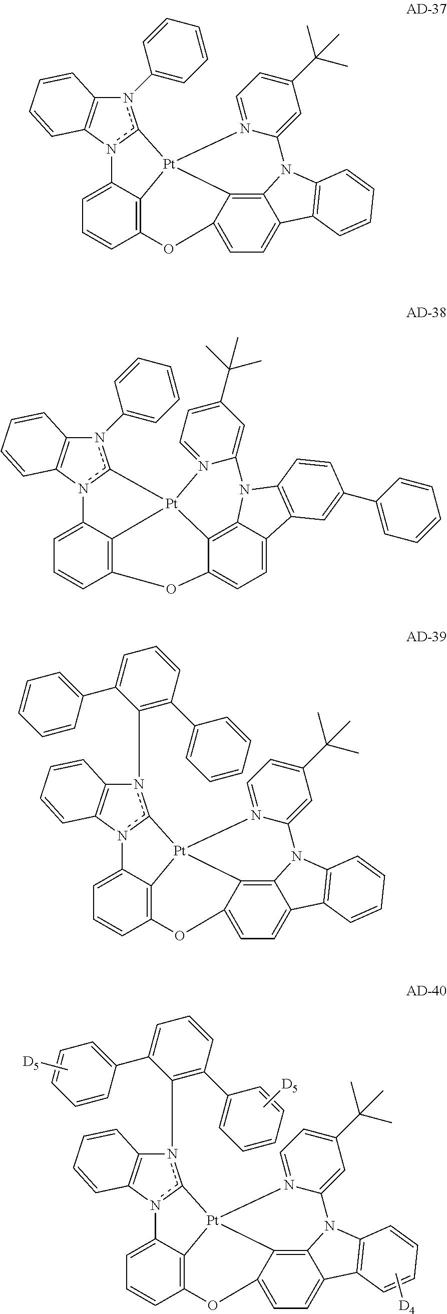

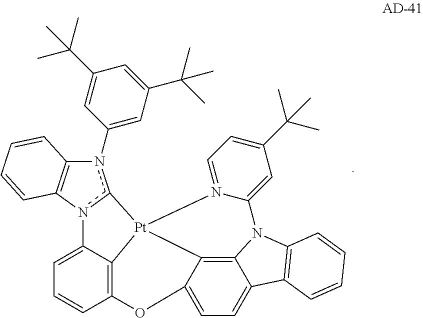

In the example compounds presented in Compound Group 3, “D” indicates deuterium.

In one or more embodiments, the light emitting element ED may include a plurality of emission layers. The plurality of emission layers may be sequentially stacked and provided, and for example, the light emitting element ED including the plurality of emission layers may be to emit white light (e.g., combined white light). The light emitting element including the plurality of emission layers may be a light emitting element having a tandem structure. When the light emitting element ED includes the plurality of emission layers, at least one emission layer EML may include the first compound represented by Formula 1 according to one or more embodiments. In some embodiments, when the light emitting element ED includes the plurality of emission layers, at least one emission layer EML may include all of the first compound, the second compound, and the third compound.

When the emission layer EML in the light emitting element ED according to one or more embodiments includes all of the first compound, the second compound, and the third compound, the third compound may be in an amount of about 0.1 wt % to about 20 wt % with respect to a total weight of the first compound, the second compound, and the third compound. However, embodiments of the present disclosure are not limited thereto. When the amount of the third compound satisfies the above-described ratio, energy transfer from the first compound and the second compound to the third compound may be greater, and accordingly, light emitting efficiency and element service life may be increased.

In the emission layer EML, an amount of the first compound and the second compound may be the remainder excluding the above-described weight of the third compound. For example, in one or more embodiments, in the emission layer EML, the amount of the first compound and the second compound may be about 65 wt % to about 95 wt % with respect to the total weight of the first compound, the second compound, and the third compound.

A weight ratio of the first compound and the second compound with respect to the total weight of the first compound and the second compound may be about 3:7 to about 7:3.

When the amount of the first compound and the second compound satisfies the above-described ratio, charge balance in the emission layer EML may be improved to increase light emitting efficiency and element service life. When the amount of the first compound and the second compound is out of the above-described ratio range, the charge balance in the emission layer EML may be impaired to reduce light emitting efficiency and easily deteriorate an element.

In the light emitting device ED of one or more embodiments, the emission layer EML may further include at least one of an anthracene derivative, a pyrene derivative, a fluoranthene derivative, a chrysene derivative, a dihydrobenzanthracene derivative, or a triphenylene derivative. For example, in some embodiments, the emission layer EML may include one or more anthracene derivatives and/or one or more pyrene derivatives.

In each light emitting element ED of one or more embodiments illustrated in FIGS. 3 to 6, the emission layer EML may further include a suitable host and dopant besides the above-described host and dopant, and for example, in some embodiments, the emission layer EML may include a compound represented by Formula E-1. The compound represented by Formula E-1 may be utilized as a fluorescent host material.

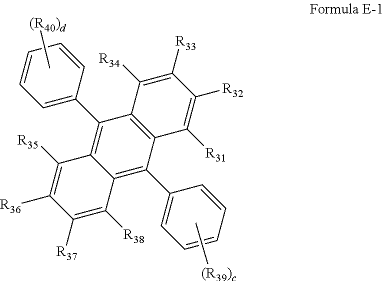

In Formula E-1, R₃₁ to R₄₀ may each independently be hydrogen, deuterium, a halogen, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In some embodiments, one or more selected from R₃₁ to R₄₀ may each independently be bonded to an adjacent group to form a saturated hydrocarbon ring or an unsaturated hydrocarbon ring, a saturated heterocycle, or an unsaturated heterocycle.

In Formula E-1, c and d may each independently be an integer of 0 to 5.

The compound represented by Formula E-1 may be any one selected from among Compound E1 to Compound E19:

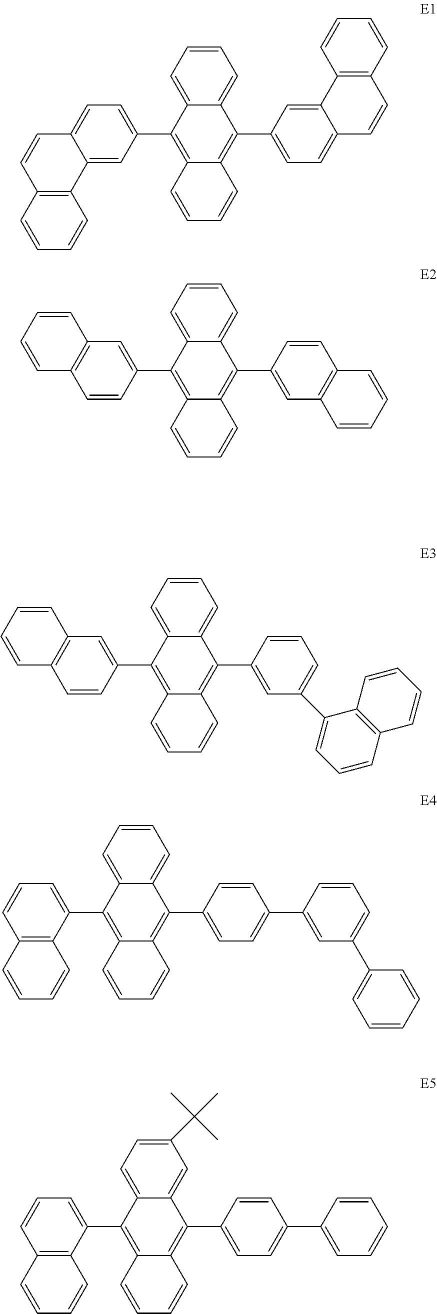

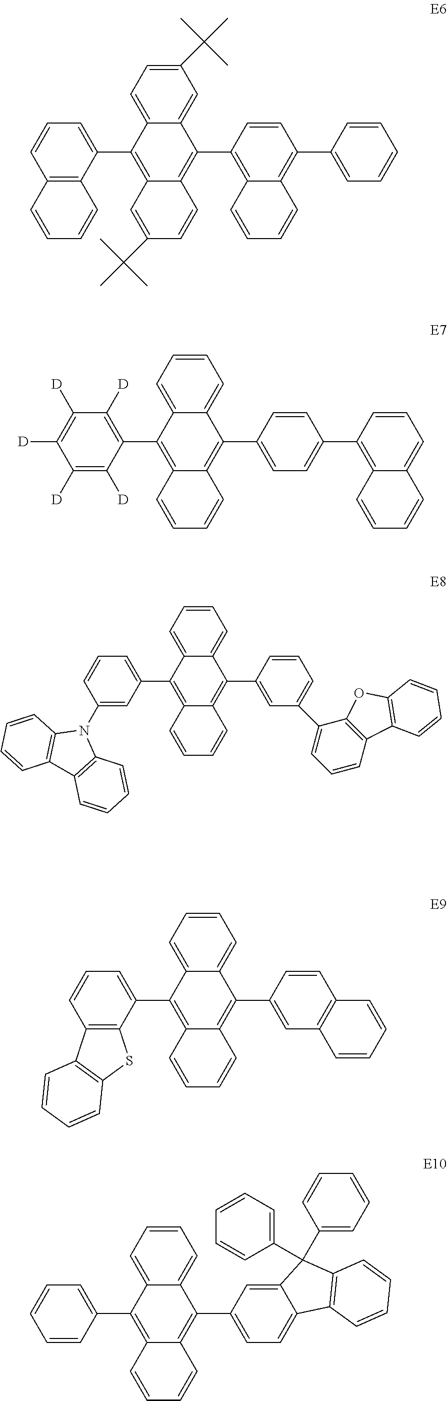

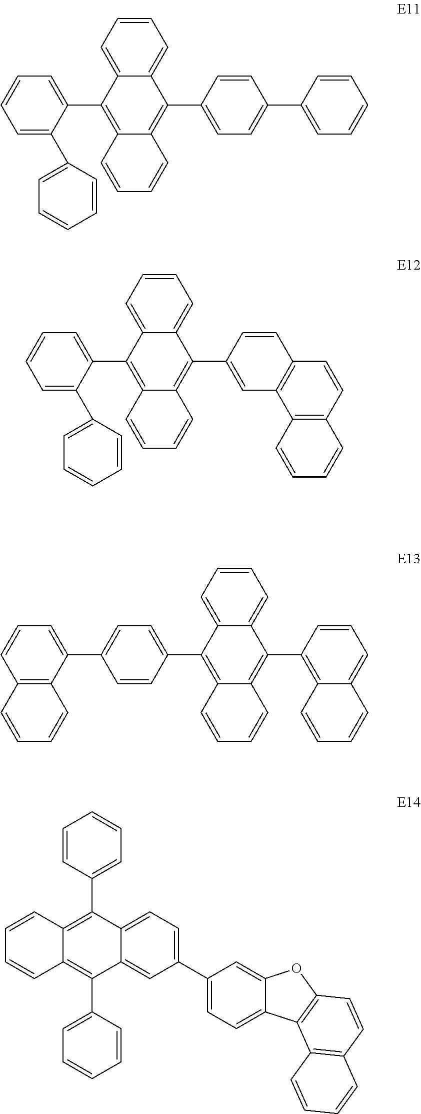

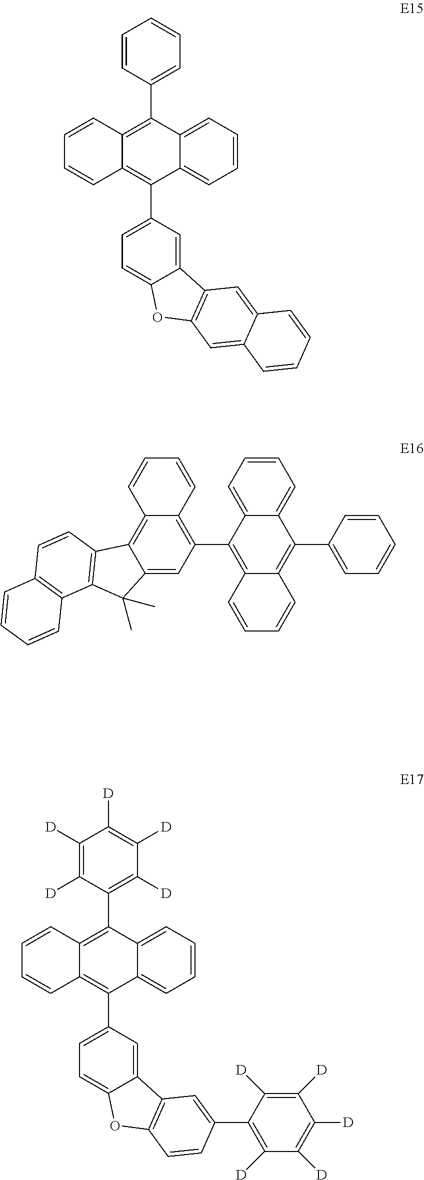

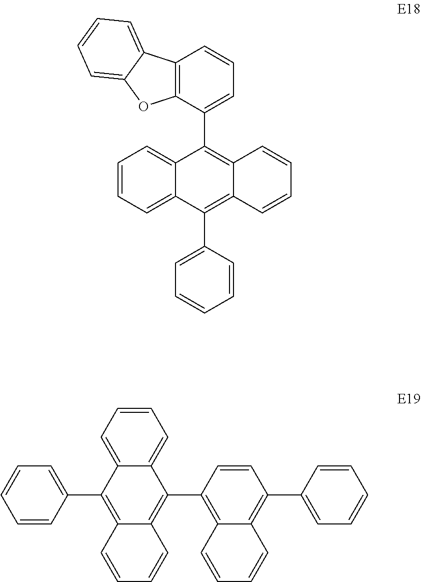

In one or more embodiments, the emission layer EML may include a compound represented by Formula E-2a or Formula E-2b. The compound represented by Formula E-2a or Formula E-2b may be utilized as a phosphorescent host material.

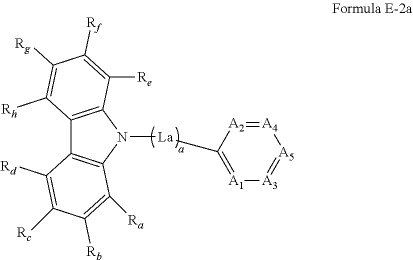

In Formula E-2a, a may be an integer of 0 to 10, and Lₐ may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of Lₐ's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be hydrogen, deuterium, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In one or more embodiments, one or more selected from among Rₐ to Rᵢ may each independently be bonded to an adjacent group to form a hydrocarbon ring or a heterocycle containing N, O, S, etc., as a ring-forming atom.

In some embodiments, in Formula E-2a, two or three selected from among A₁ to A₅ may be N, and the rest may be CRᵢ.

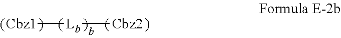

In Formula E-2b, Cbz1 and Cbz2 may each independently be an unsubstituted carbazole group, or a carbazole group substituted with an aryl group having 6 to 30 ring-forming carbon atoms. Lb may be a direct linkage, a substituted or unsubstituted arylene group having 6 to 30 ring-forming carbon atoms, or a substituted or unsubstituted heteroarylene group having 2 to 30 ring-forming carbon atoms. b may be an integer of 0 to 10, and when b is an integer of 2 or more, a plurality of Lb's may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be any one selected from among compounds of Compound Group E-2. However, the compounds listed in Compound Group E-2 are mere examples, and the compound represented by Formula E-2a or Formula E-2b is not limited to those represented in Compound Group E-2.

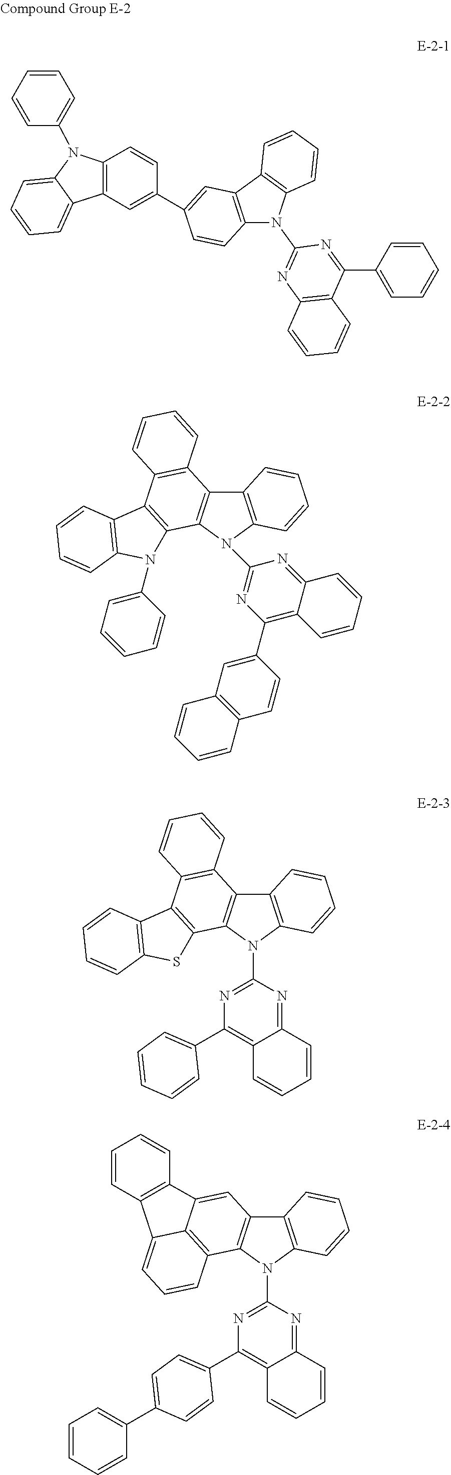 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1 ; c1ccc(-c2ccc(-c3nc(-n4c5ccccc5c5cc6c7c(cccc7c54)-c4ccccc4-6)nc4ccccc34)cc2)cc1]

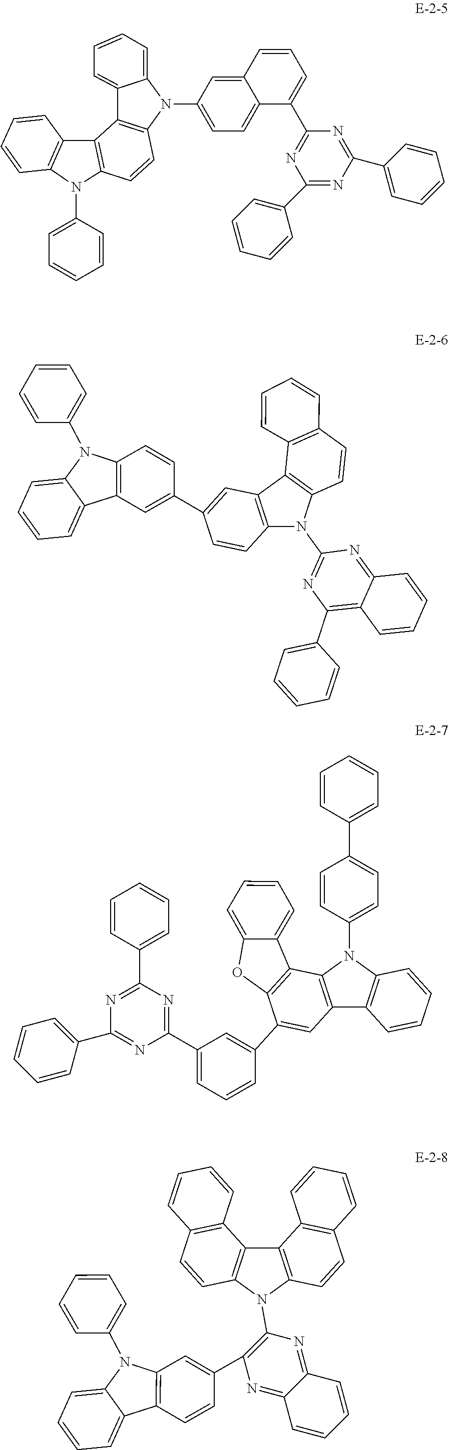 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

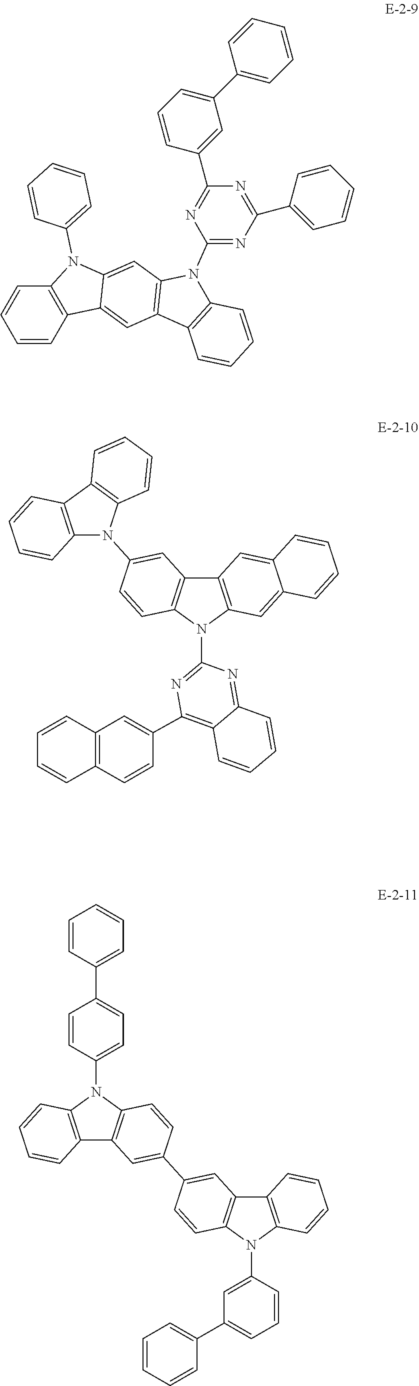 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

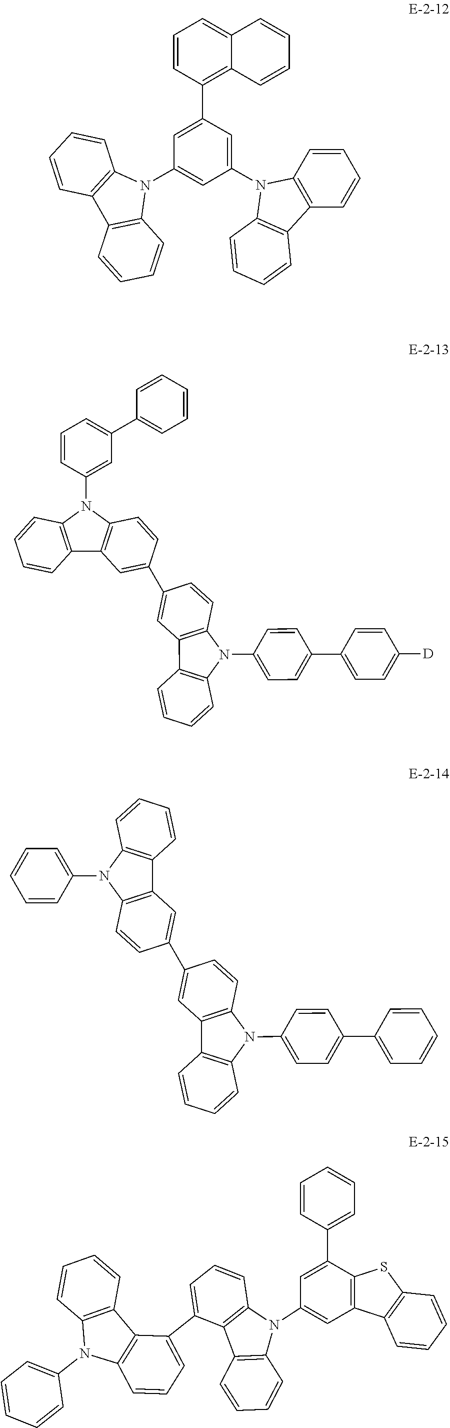 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

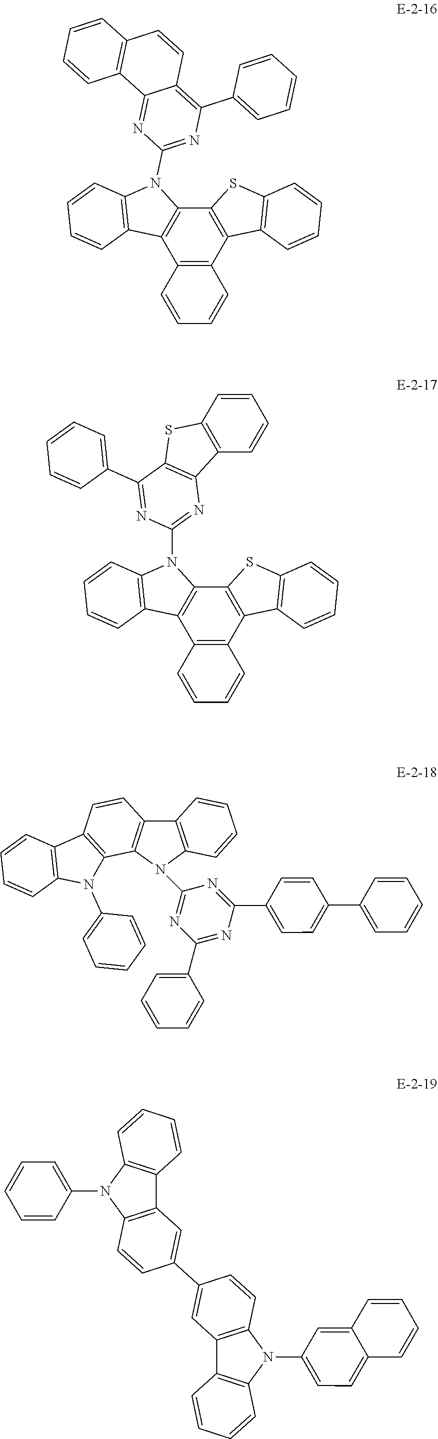 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

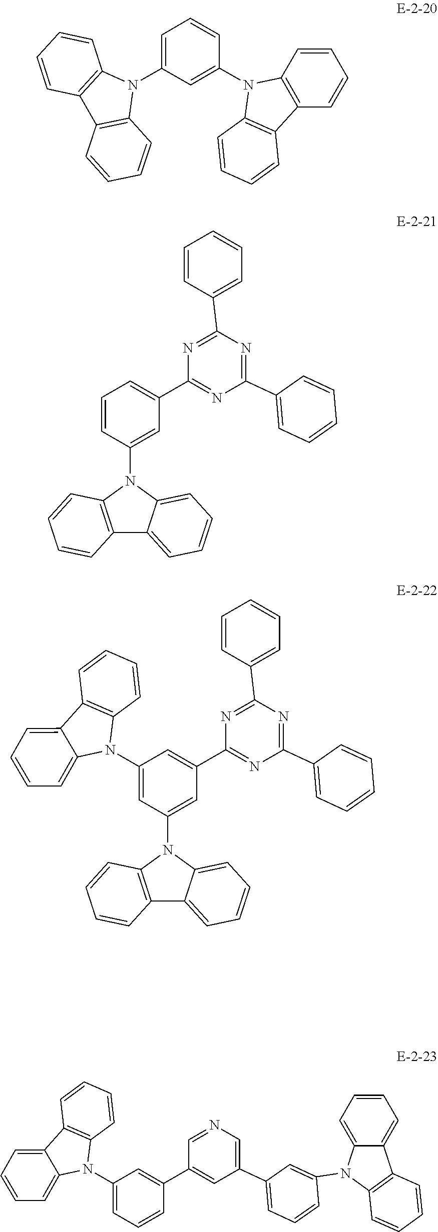 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

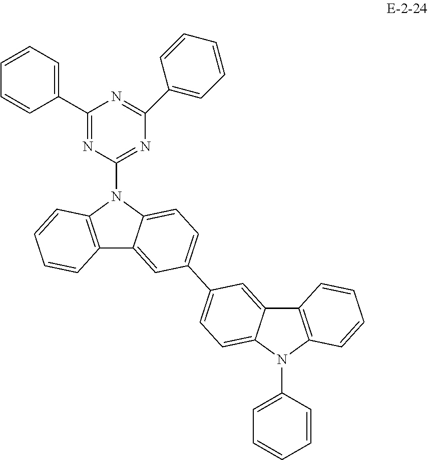 [SMILES: c1ccc(-c2nc(-c3ccccc3)nc(-n3c4ccccc4c4cc(-c5ccc6c(c5)c5ccccc5n6-c5ccccc5)ccc43)n2)cc1]

In one or more embodiments, the emission layer EML may further include a material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazol-2-yl)benzene (TPBi). However, embodiments of the present disclosure are not limited thereto, for example, tris(8-hydroxyquinolinato)aluminum (Alq3), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), etc. may be utilized as a host material.

In one or more embodiments, the emission layer EML may include a compound represented by Formula M-a. The compound represented by Formula M-a may be utilized as a phosphorescent dopant material.

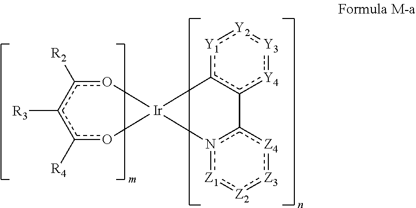

In Formula M-a, Y₁ to Y₄ and Z₁ to Z₄ may each independently be CR₁ or N, R₁ to R₄ may each independently be hydrogen, deuterium, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. In Formula M-a, m is 0 or 1, and n is 2 or 3. In Formula M-a, when m is 0, n is 3, and when m is 1, n is 2.

The compound represented by Formula M-a may be utilized as a phosphorescent dopant.

The compound represented by Formula M-a may be any one selected from among Compound M-a1 to Compound M-a25. However, Compounds M-a1 to M-a25 are mere examples, and the compound represented by Formula M-a is not limited to those represented by Compounds M-a1 to M-a25.

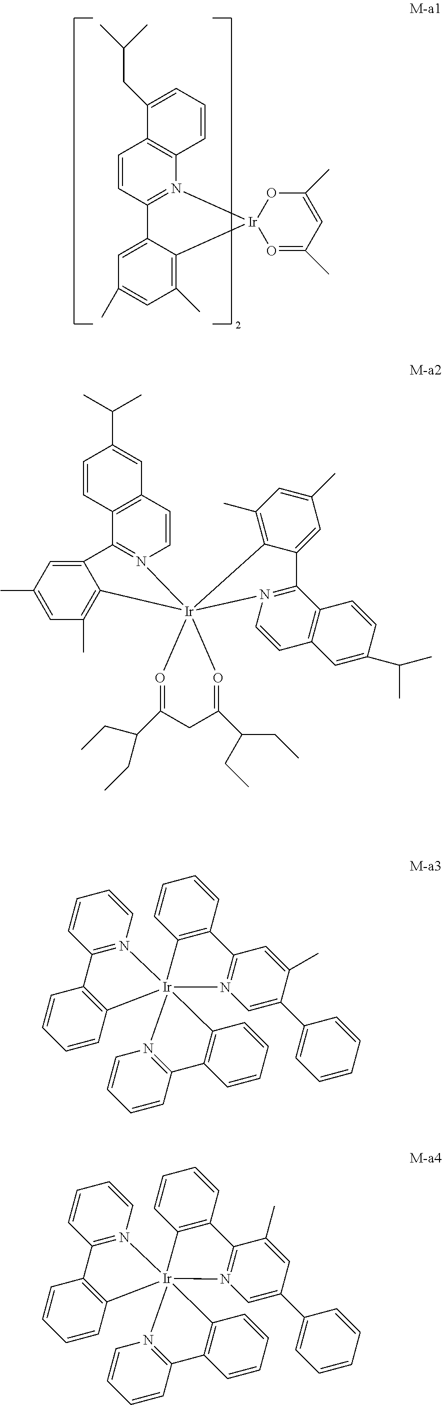

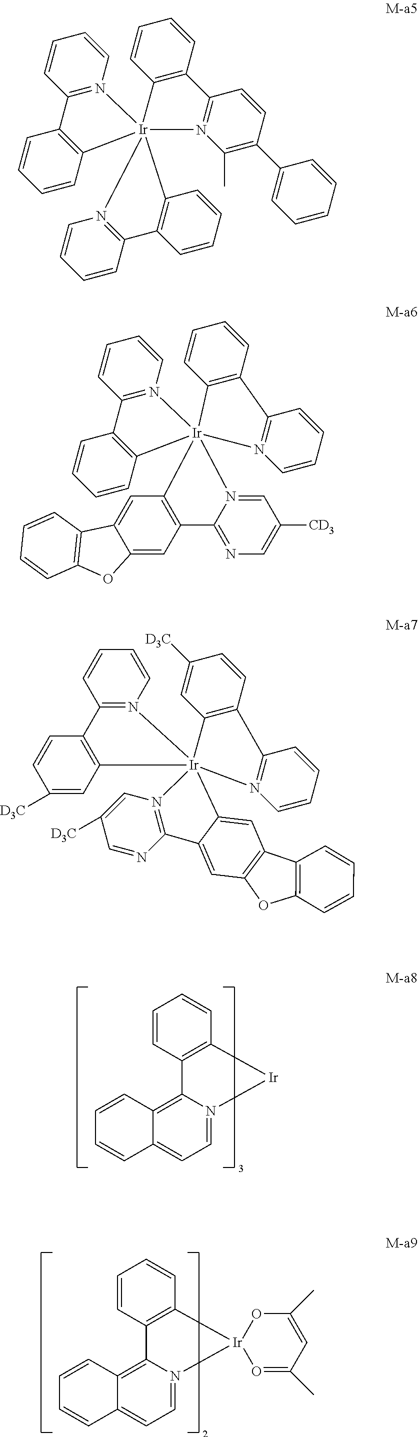

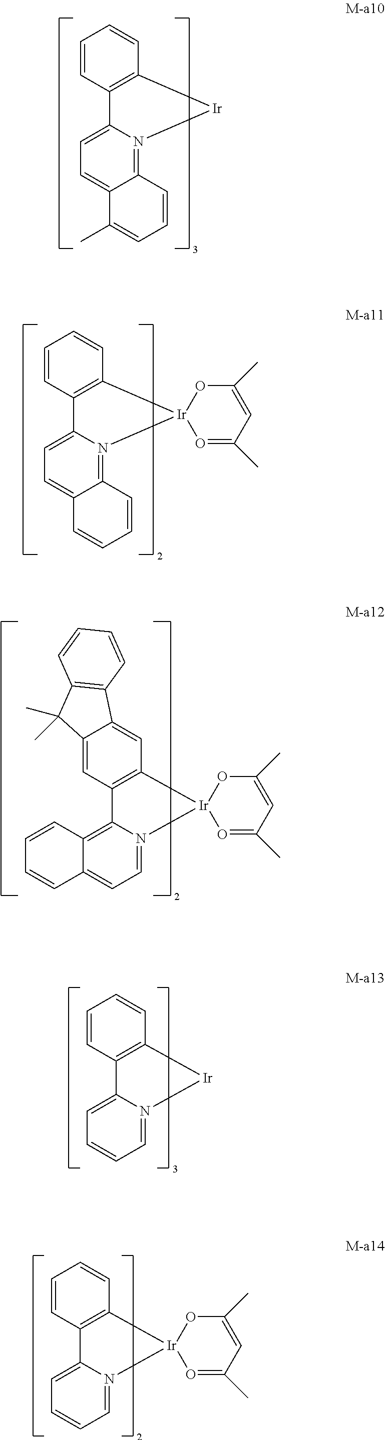

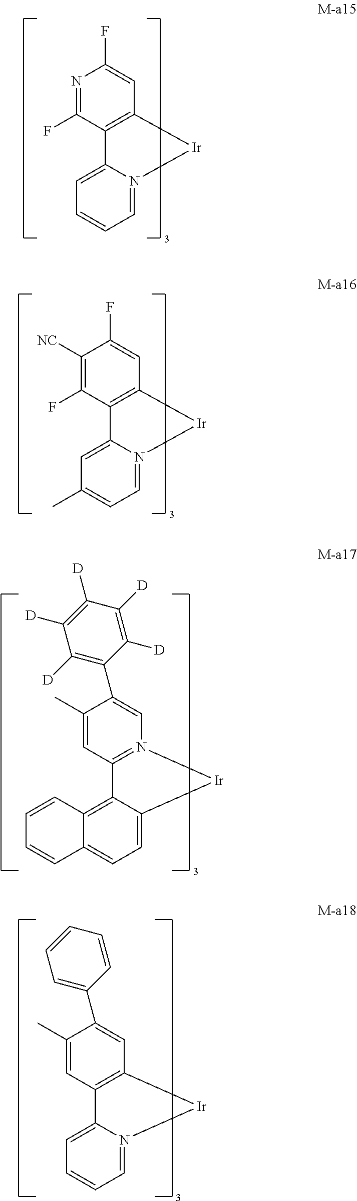

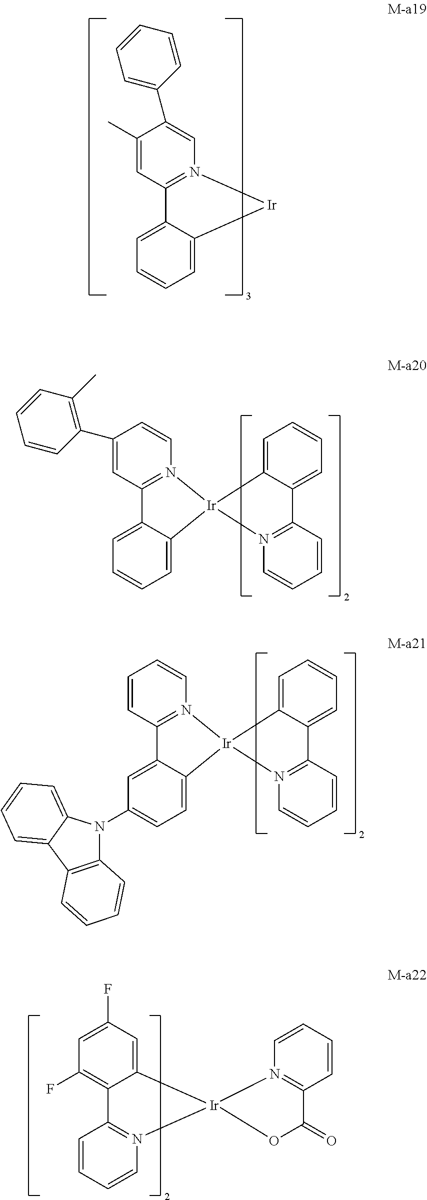

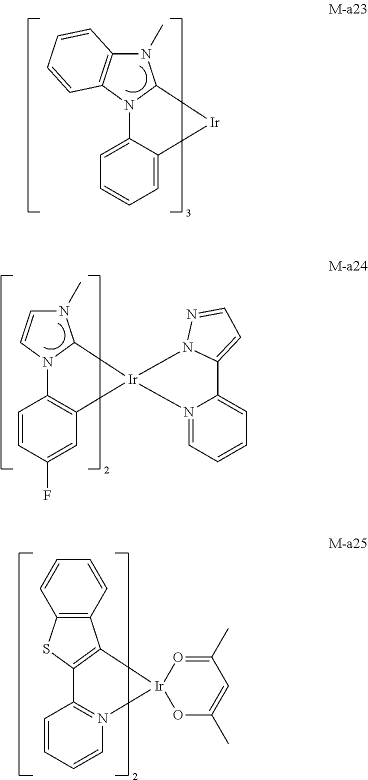

In one or more embodiments, the emission layer EML may include a compound represented by any one selected from among Formula F-a to Formula F-c. The compound represented by Formula F-a to Formula F-c may be utilized as a fluorescence dopant material.

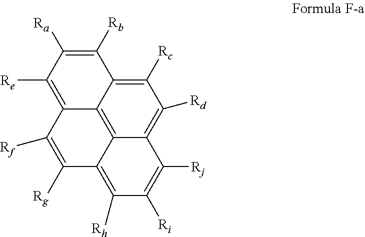

In Formula F-a, two selected from among Rₐ to Rⱼ may each independently be substituted with *-NAr₁Ar₂. The others, which are not substituted with *-NArqAr₂, among Rₐ to Rⱼ may each independently be hydrogen, deuterium, a halogen, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *-NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, in some embodiments, at least one of Ar₁ or Ar₂ may be a heteroaryl group containing 0 or S as a ring-forming atom.

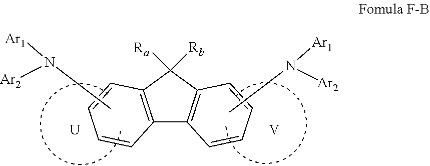

In Formula F-b, Rₐ and Rb may each independently be hydrogen, deuterium, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. In some embodiments, at least one selected from among Ar₁ to Ar₄ may be a heteroaryl group containing O or S as a ring-forming atom.

In Formula F-b, the number of rings represented by U and V may each independently be 0 or 1. For example, in Formula F-b, it refers to that when the number of U or V is 1, one ring constitutes a fused ring at a portion indicated by U or V, and when the number of U or V is 0, a ring indicated by U or V does not exist. For example, when the number of U is 0 and the number of V is 1, or when the number of U is 1 and the number of V is 0, the fused ring having a fluorene core in Formula F-b may be a cyclic compound having four rings. In some embodiments, when each number of U and V is 0, the fused ring in Formula F-b may be a cyclic compound having three rings. In some embodiments, when each number of U and V is 1, the fused ring having a fluorene core in Formula F-b may be a cyclic compound having five rings.

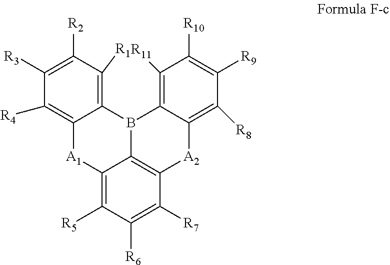

In Formula F-c, A₁ and A₂ may each independently be O, S, Se, or NRₘ, and μm may be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be bonded to an adjacent group to form a ring.

In Formula F-c, A₁ and A₂ may each independently be bonded to substituents of an adjacent ring to form a fused ring. For example, when A₁ and A₂ may each independently be NRₘ, A₁ may be bonded to R₄ or R₅ to form a ring. In some embodiments, A₂ may be bonded to R₇ or R₈ to form a ring.

In one or more embodiments, the emission layer EML may further include, as a suitable dopant material, one or more selected from a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1-dipyrene, 1,4-dipyrenylbenzene, 1,4-bis(N,N-diphenylamino)pyrene), etc.

In one or more embodiments, the emission layer EML may further include a suitable phosphorescence dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be utilized as a phosphorescent dopant. For example, in some embodiments, iridium(III) bis(4,6-difluorophenylpyridinato-N,C2) (Flrpic), bis(2,4-difluorophenylpyridinato)-tetrakis(1-pyrazolyl)borate iridium(III) (Fir6), or platinum octaethyl porphyrin (PtOEP) may be utilized as a phosphorescent dopant. However, embodiments of the present disclosure are not limited thereto.

In one or more embodiments, the emission layer EML may include a quantum dot material. In one or more embodiments, the quantum dot material may have a core/shell structure. The core of the quantum dot may be selected from a Group II-VI compound, a Group III-VI compound, a Group I-III-IV compound, a Group III-V compound, a Group III-II-V compound, a Group IV-VI compound, a Group IV element, a Group IV compound, and one or more combinations thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and one or more mixtures (and/or combinations) thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and one or more mixtures (and/or combinations) thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and one or more mixtures (and/or combinations) thereof.

The Group III-VI compound may include a binary compound such as In₂S₃ and/or In₂Se₃, a ternary compound such as InGaS₃ and/or InGaSe₃, or any combination thereof.

The Group I-III-VI compound may be selected from a ternary compound selected from the group consisting of AgInS, AgInS₂, CuInS, CuInS₂, AgGaS₂, CuGaS₂ CuGaO₂, AgGaO₂, AgAlO₂, and one or more mixtures (and/or combinations) thereof, and/or a quaternary compound such as AgInGaS₂ and/or CulnGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and one or more mixtures (and/or combinations) thereof; a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and one or more mixtures (and/or combinations) thereof; and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and one or more mixtures (and/or combinations) thereof. In some embodiments, the Group III-V compound may further include a Group II metal. For example, InZnP, etc., may be selected as a Group III-II-V compound.

The Group IV-VI compound may be selected from the group consisting of a binary compound selected from the group consisting of SnS, SnSe, SnTe, PbS, PbSe, PbTe, and one or more mixtures (and/or combinations) thereof: a ternary compound selected from the group consisting of SnSeS, SnSeTe, SnSTe, PbSeS, PbSeTe, PbSTe, SnPbS, SnPbSe, SnPbTe, and one or more mixtures (and/or combinations) thereof; and a quaternary compound selected from the group consisting of SnPbSSe, SnPbSeTe, SnPbSTe, and one or more mixtures (and/or combinations) thereof. The Group IV element may be selected from the group consisting of Si, Ge, and one or more mixtures (and/or combinations) thereof. The Group IV compound may be a binary compound selected from the group consisting of SiC, SiGe, and one or more mixtures (and/or combinations) thereof.

Each element included in a polynary compound such as the binary compound, the ternary compound, or the quaternary compound may be present in a particle with a substantially uniform or non-substantially uniform concentration distribution. For example, the formulae above refer to the types (kinds) of elements included in the compounds, and the elemental ratio in the compound may be different. For example, AgInGaS₂ may refer to AgInxGa₁₋ₓS₂ (where x is a real number of 0 to 1).

In some embodiments, the quantum dot may have a single structure or a double structure of core-shell in which the concentration of each element included in the quantum dot is substantially uniform. For example, in some embodiments, the material included in the core may be different from the material included in the shell.

The shell of the quantum dot may serve as a protection layer to prevent or reduce the chemical deformation of the core to maintain semiconductor properties, and/or a charging layer to impart electrophoresis properties to the quantum dot. The shell may be a single layer or multiple layers. An interface between the core and the shell may have a concentration gradient in which the concentration of an element present in the shell becomes lower towards the center.

In some embodiments, the quantum dot may have the above-described core/shell structure including a core containing nanocrystals and a shell around (e.g., surrounding) the core. An example of the shell of the quantum dots may include a metal or non-metal oxide, a semiconductor compound, or a combination thereof.

For example, the metal or non-metal oxide for the shell may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, and/or a ternary compound such as MgAl₂O₄, CoFe₂O₄, NiFe₂O₄, and/or CoMn₂O₄, but embodiments of the present disclosure are not limited thereto.

Also, examples of the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

Each element included in a polynary compound such as the binary compound, or the ternary compound may be present in a particle with a substantially uniform or non-substantially uniform concentration distribution. For example, the formulae refer to the types (kinds) of elements included in the compounds, and the elemental ratio in the compound may be different.

The quantum dot may have a full width of half maximum (FWHM) of a light emitting wavelength spectrum (e.g., an emission spectrum) of about 45 nm or less, about 40 nm or less, or about 30 nm or less, and color purity or color reproducibility of the light emitting element may be improved within the above range. In some embodiments, light emitted through such quantum dot is emitted in all directions so that a wide viewing angle may be improved.

In some embodiments, although the form of the quantum dot is not particularly limited as long as it is a form commonly utilized in the art, the quantum dot in the form of spherical nanoparticles, pyramidal nanoparticles, multi-arm nanoparticles, or cubic nanoparticles, nanotubes, nanowires, nanofibers, nanoplate particles, etc. may be utilized.

As the size of the quantum dot is adjusted or the elemental ratio in the quantum dot compound is adjusted, it may control the energy band gap of the quantum dot, and thus light in one or more suitable wavelength ranges may be obtained in a quantum dot emission layer. Therefore, when the quantum dot as above (e.g., utilizing different sizes of quantum dots or different elemental ratios in the quantum dot compound) are utilized, the light emitting device, which emits light in one or more suitable wavelengths, may be implemented. For example, the adjustment of the size of the quantum dot and/or the elemental ratio in the quantum dot compound may be performed to enable the quantum dots to emit red, green, and/or blue light. In some embodiments, the quantum dots may be configured to emit white light by combining one or more suitable colors of light.

In each of the light emitting elements ED of one or more embodiments illustrated in FIGS. 3 to 6, the electron transport region ETR may be provided on the emission layer EML. The electron transport region ETR may include at least one of a hole blocking layer HBL, an electron transport layer ETL, or an electron injection layer EIL, but embodiments of the present disclosure are not limited thereto.

The electron transport region ETR may have a single layer formed of a single material, a single layer formed of a plurality of different materials, or a multilayer structure including a plurality of layers formed of a plurality of different materials.

For example, in some embodiments, the electron transport region ETR may have a single layer structure of an electron injection layer EIL or an electron transport layer ETL, or may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order (e.g., in the stated order) from the emission layer EML, but embodiments of the present disclosure are not limited thereto. The electron transport region ETR may have a thickness, for example, from about 1,000 Å to about 1,500 Å.

The electron transport region ETR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-2:

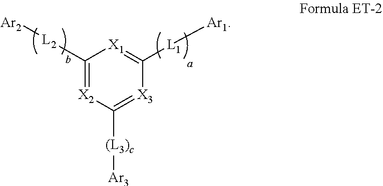

In Formula ET-2, at least one selected from among X₁ to X₃ may be N, and the rest may be CRₐ. Rₐ may be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

In one or more embodiments, the electron transport region ETR may include an anthracene-based compound. However, embodiments of the present disclosure are not limited thereto, for example, in some embodiments, the electron transport region ETR may include, for example, at least one selected from among tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), and one or more mixtures thereof.

In one or more embodiments, the electron transport region ETR may include at least one selected from among Compound ET1 to Compound ET36:

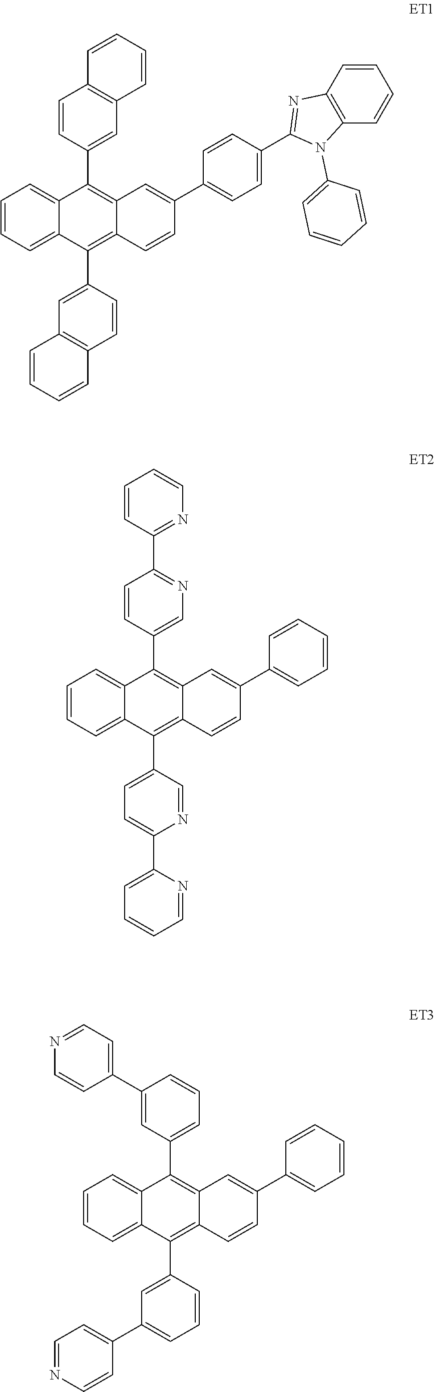 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

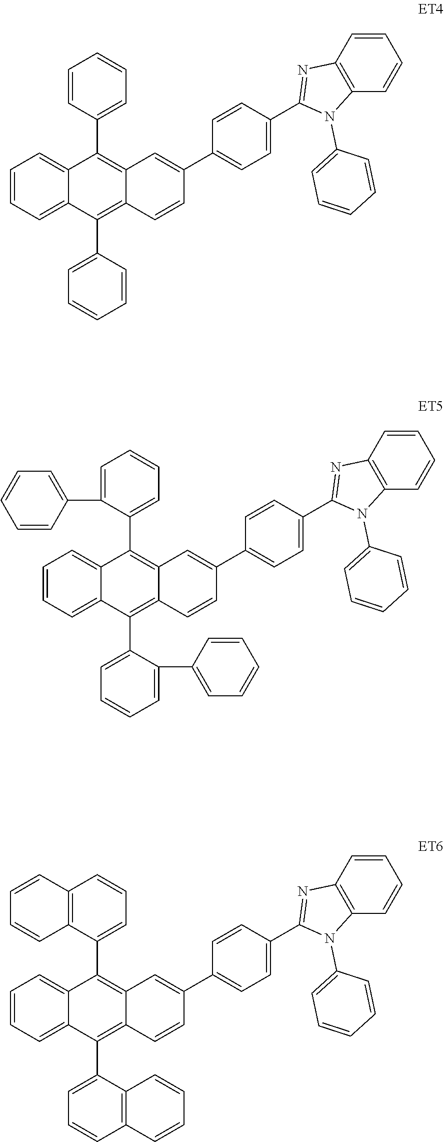 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

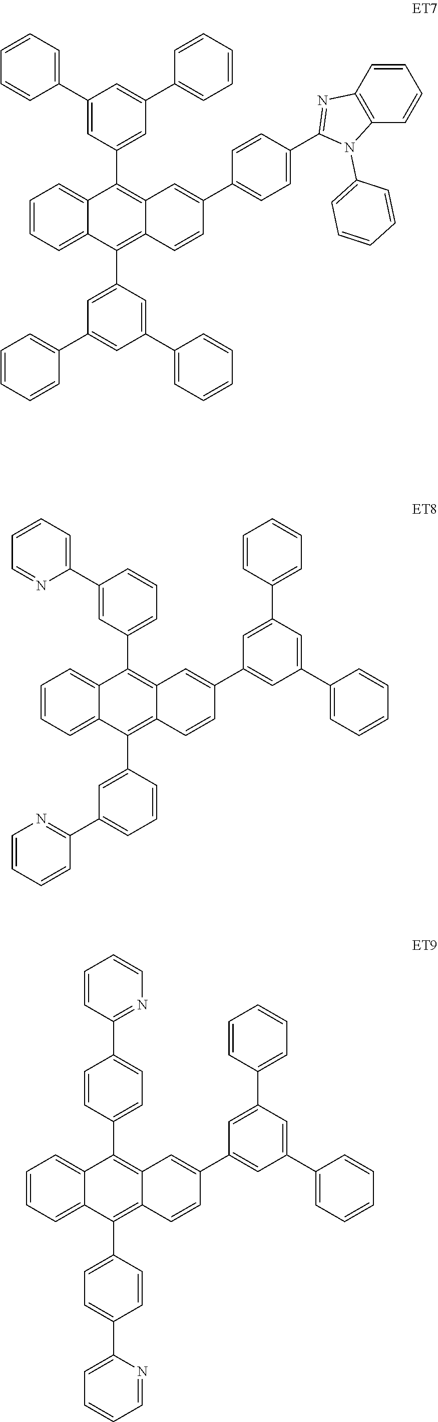 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

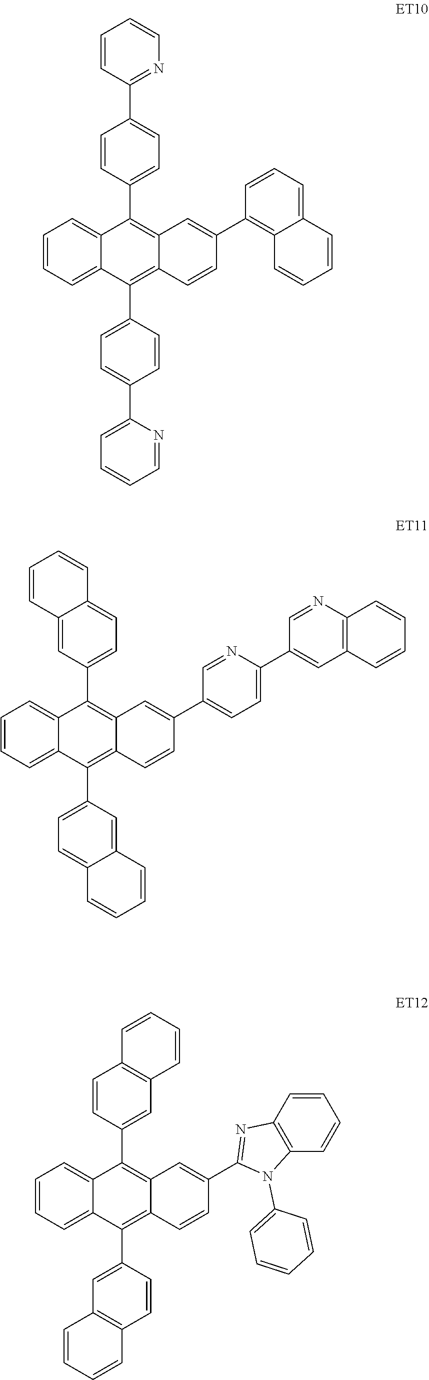 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

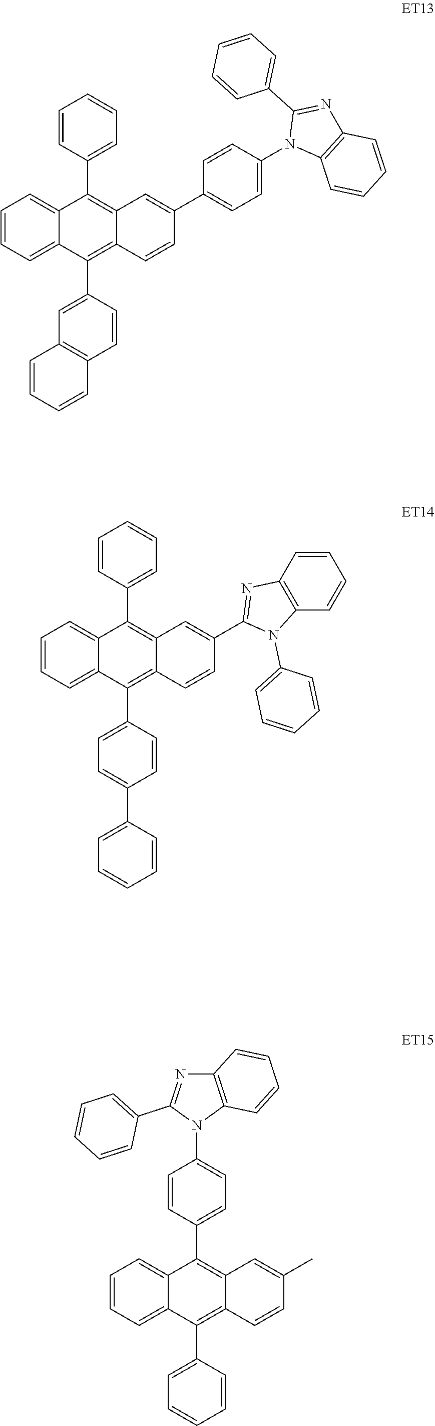 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

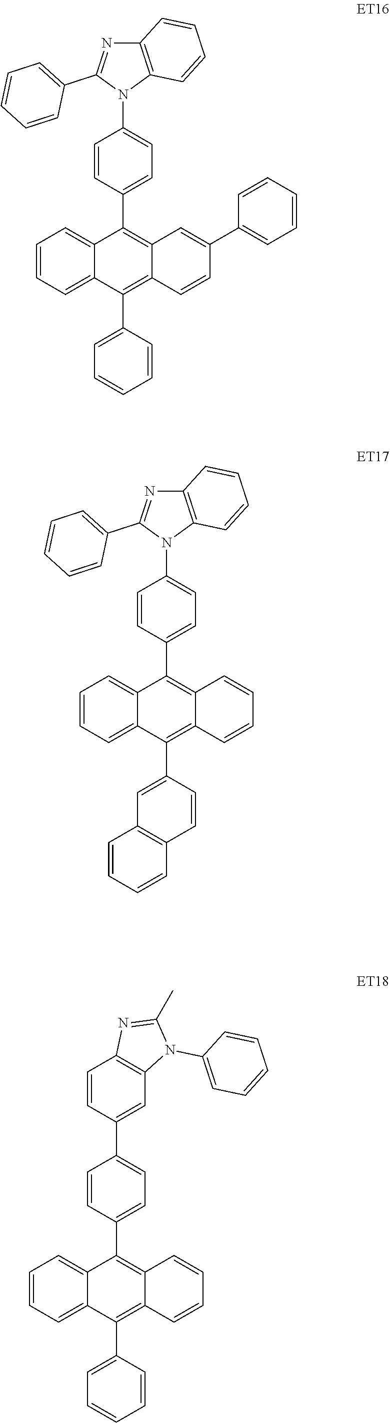 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

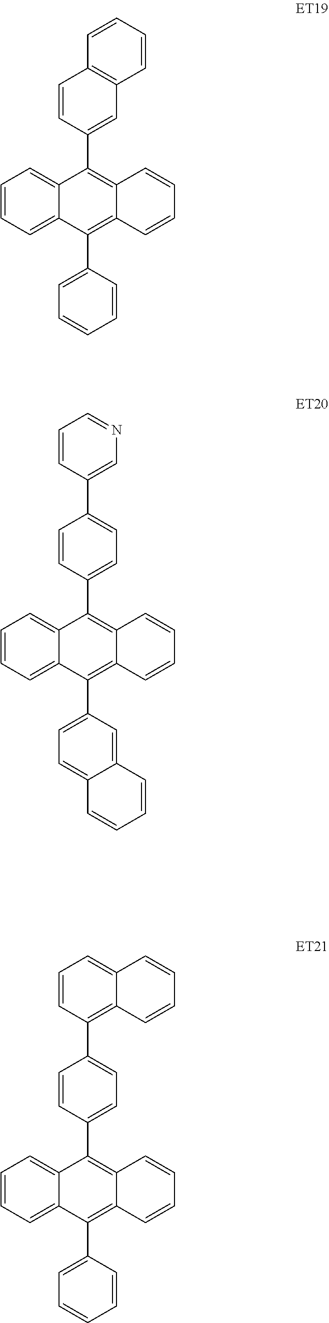

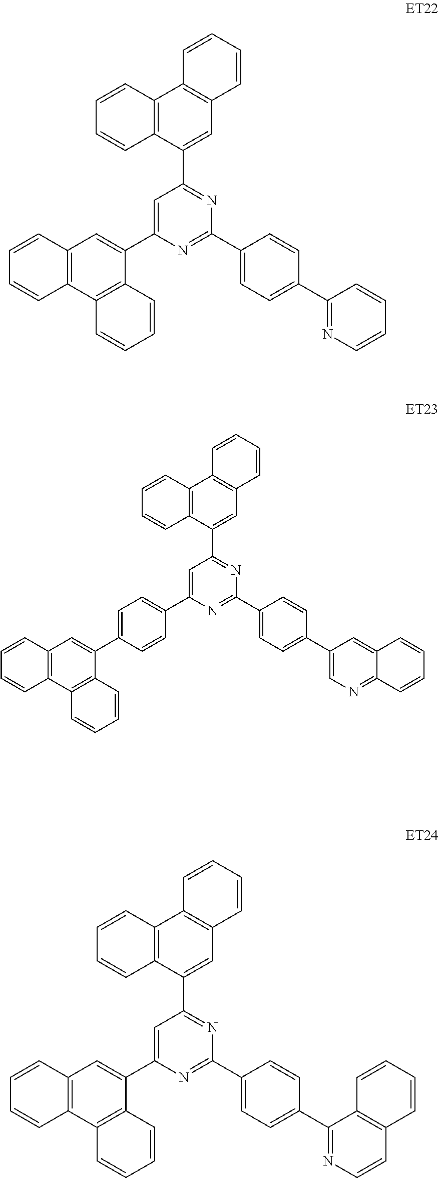 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

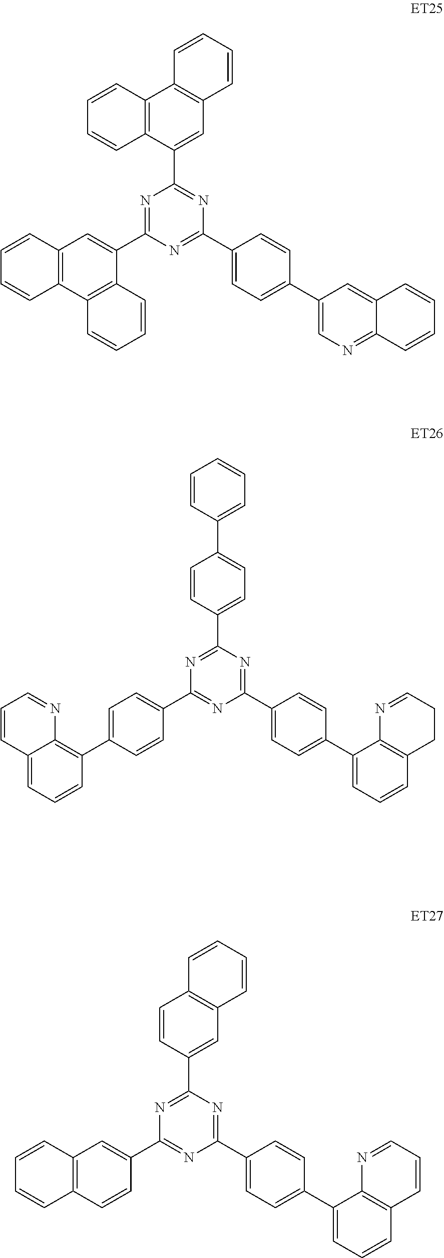 [SMILES: c1ccc2ncc(-c3ccc(-c4nc(-c5cc6ccccc6c6ccccc56)nc(-c5cc6ccccc6c6ccccc56)n4)cc3)cc2c1 ; C1=Nc2c(cccc2-c2ccc(-c3nc(-c4ccc(-c5ccccc5)cc4)nc(-c4ccc(-c5cccc6cccnc56)cc4)n3)cc2)CC1 ; c1ccc2cc(-c3nc(-c4ccc(-c5cccc6cccnc56)cc4)nc(-c4ccc5ccccc5c4)n3)ccc2c1]

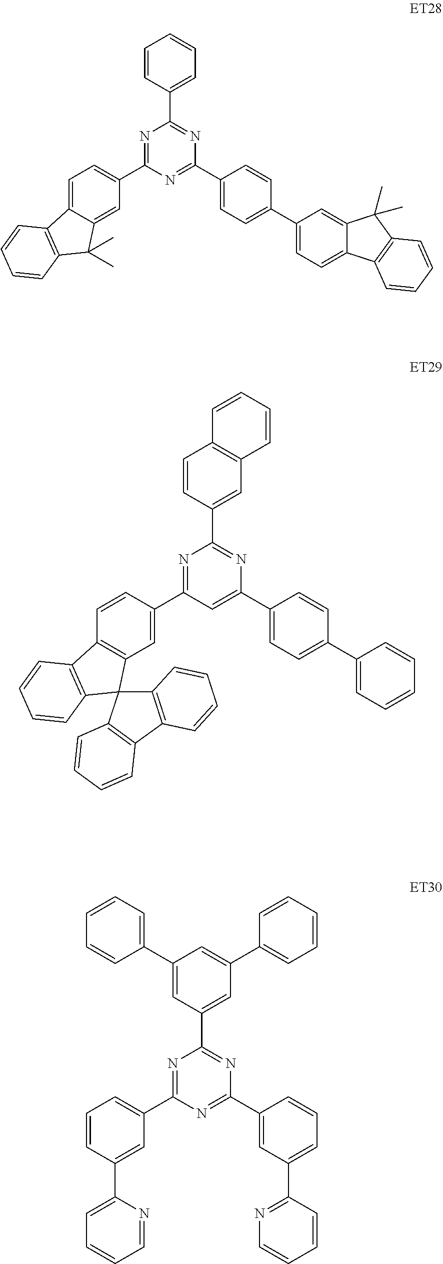 [SMILES: CC1(C)c2ccccc2-c2ccc(-c3ccc(-c4nc(-c5ccccc5)nc(-c5ccc6c(c5)C(C)(C)c5ccccc5-6)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

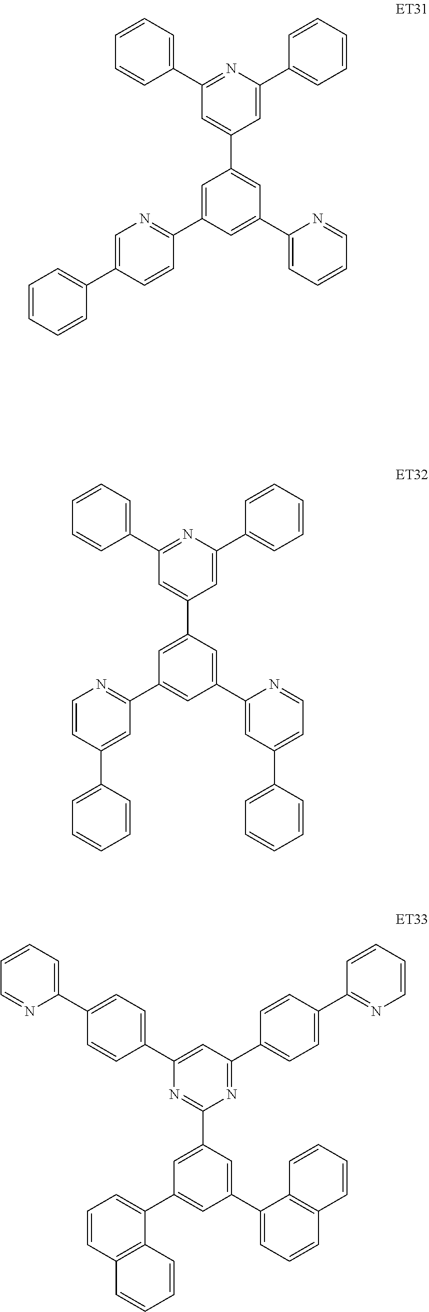 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

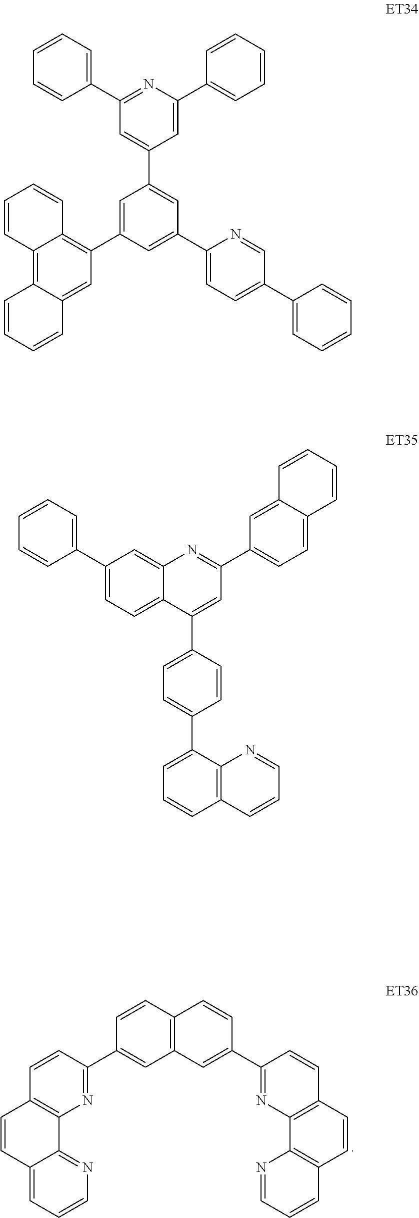 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and/or KI, a lanthanide metal such as Yb, or a co-deposited material of the metal halide and the lanthanide metal. For example, in some embodiments, the electron transport region ETR may include KI:Yb, RbI:Yb, LiF:Yb, etc., as a co-deposited material. In some embodiments, the electron transport region ETR may be formed utilizing a metal oxide such as Li₂O and/or BaO, or 8-hydroxyl-lithium quinolate (Liq), etc., but embodiments of the present disclosure are not limited thereto. In some embodiments, the electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, and/or a metal stearate.

In one or more embodiments, the electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but embodiments of the present disclosure are not limited thereto.

The electron transport region ETR may include the above-described compounds of the electrode transport region in at least one of an electron injection layer EIL, an electron transport layer ETL, or a hole blocking layer HBL.

When the electron transport region ETR includes an electron transport layer ETL, the electron transport layer ETL may have a thickness of about 100 Å to about 1,000 Å, for example, about 150 Å to about 500 Å. When the thickness of the electron transport layer ETL satisfies the aforementioned range, satisfactory electron transport characteristics may be obtained without a substantial increase in driving voltage. When the electron transport region ETR includes an electron injection layer EIL, the electron injection layer EIL may have a thickness of about 1 Å to about 100 Å, for example, about 3 Å to about 90 Å. When the thickness of the electron injection layer EIL satisfies the above-described range, satisfactory electron injection characteristics may be obtained without a substantial increase in driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/AI, Mo, Ti, Yb, W, one or more compounds thereof, or one or more mixtures thereof (e.g., AgMg, AgYb, or MgYb). In some embodiments, the second electrode EL2 may have a multilayer structure including a reflective film or a transflective film formed of one or more of the above-described materials, and a transparent conductive film formed of ITO, IZO, ZnO, ITZO, etc. For example, the second electrode EL2 may include one selected from the above-described metal materials, one or more combinations of at least two metal materials of the above-described metal materials, or one or more oxides of the above-described metal materials, and/or the like.

In some embodiments, the second electrode EL2 may be connected with an auxiliary electrode. When the second electrode EL2 is connected with the auxiliary electrode, the resistance of the second electrode EL2 may be decreased.

In some embodiments, a capping layer CPL may further be disposed on the second electrode EL2 of the light emitting element ED of one or more embodiments. The capping layer CPL may include a multilayer or a single layer.

In one or more embodiments, the capping layer CPL may be an organic layer and/or an inorganic layer. For example, in some embodiments, when the capping layer CPL contains an inorganic material, the inorganic material may include an alkali metal compound (e.g., LiF), an alkaline earth metal compound (e.g., MgF₂), SiON, SiNₓ, SiO_{y}, etc.

For example, in some embodiments, when the capping layer CPL includes an organic material, the organic material may include one or more of 2,2′-dimethyl-N,N′-di-[(1-naphthyl)-N,N′-diphenyl]-1,1′-biphenyl-4,4′-diamine (a-NPD), NPB, TPD, m-MTDATA, Alq3, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or an epoxy resin, or acrylate such as methacrylate. However, embodiments of the present disclosure are not limited thereto, for example, the capping layer CPL may include at least one selected from among Compounds P₁ to P₅:

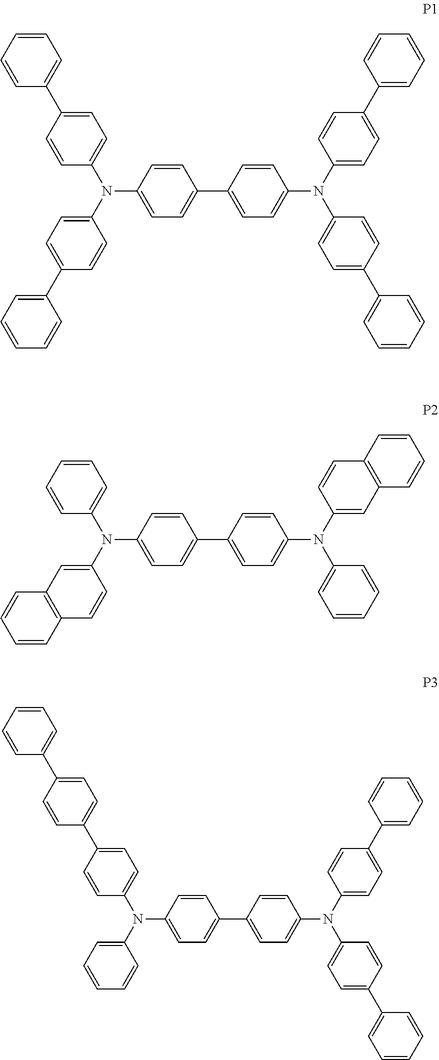 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

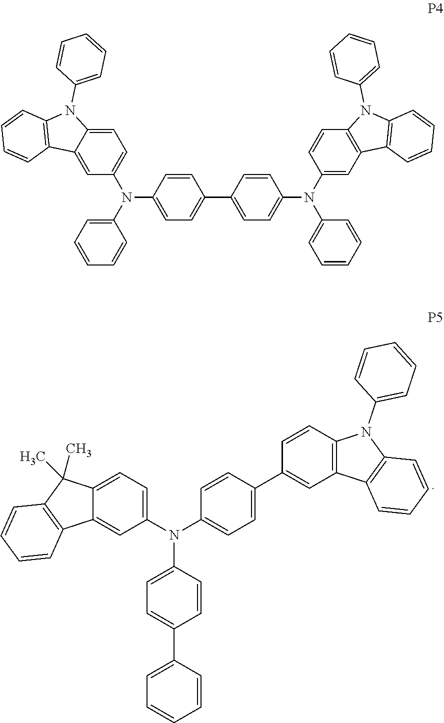 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, in one or more embodiments, the refractive index of the capping layer CPL may be about 1.6 or more with respect to light in a wavelength range of about 550 nm to about 660 nm.

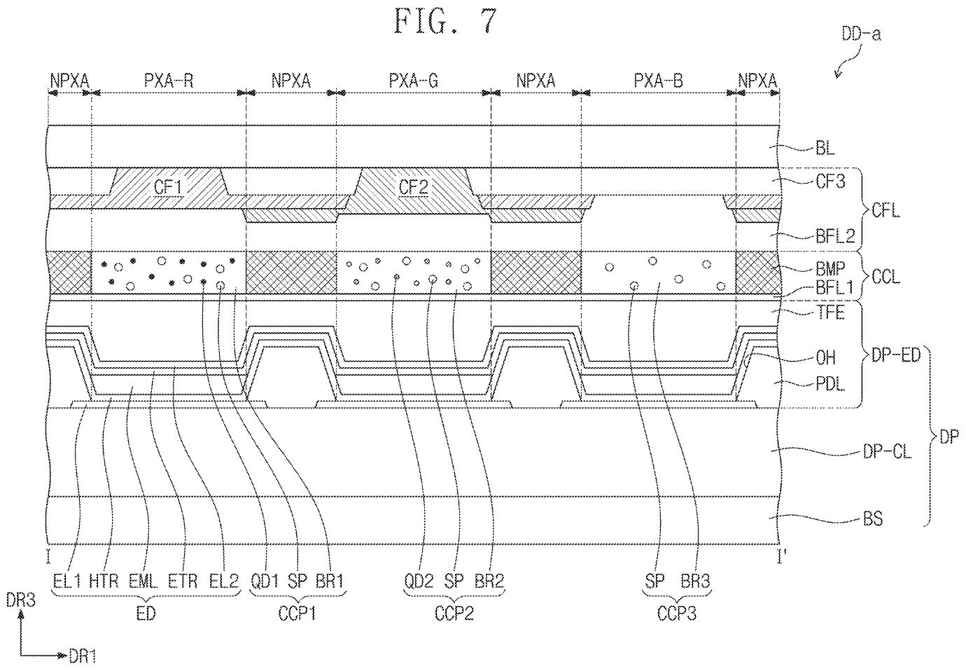

Each of FIGS. 7 to 10 is a cross-sectional view of a display apparatus according to one or more embodiments of the present disclosure. Hereinafter, in describing the display apparatuses of embodiments with reference to FIGS. 7 to 10, the duplicated features which have been described in FIGS. 1 to 6 are not described again, but their differences will be mainly described.

Referring to FIG. 7, the display apparatus DD-a according to one or more embodiments may include a display panel DP including a display device layer DP-ED, a light control layer CCL on the display panel DP, and a color filter layer CFL. In one or more embodiments illustrated in FIG. 7, the display panel DP may include a base layer BS, a circuit layer DP-CL provided on the base layer BS, and the display device layer DP-ED, and the display device layer DP-ED may include a light emitting device ED.

The light emitting device ED may include a first electrode EL1, a hole transport region HTR on the first electrode EL1, an emission layer EML on the hole transport region HTR, an electron transport region ETR on the emission layer EML, and a second electrode EL2 on the electron transport region ETR. In one or more embodiments, the structure of each of the light emitting elements of FIGS. 3 to 6 as described above may be equally applied to the structure of the light emitting device ED illustrated in FIG. 7.

The emission layer EML of the light emitting device ED included in the display device DD-a according to one or more embodiments may include the hetero compound of one or more embodiments described above.

Referring to FIG. 7, the emission layer EML may be disposed in an opening OH defined in a pixel defining film PDL. For example, the emission layer EML which is divided by the pixel defining film PDL and provided corresponding to each light emitting regions PXA-R, PXA-G, and PXA-B may be to emit light in substantially the same wavelength range. In the display apparatus DD-a of one or more embodiments, the emission layer EML may be to emit blue light. In some embodiments, the emission layer EML may be provided as a common layer in the entire light emitting regions PXA-R, PXA-G, and PXA-B.

The light control layer CCL may be disposed on the display panel DP. The light control layer CCL may include a light conversion body. The light conversion body may be a quantum dot, a phosphor, and/or the like. The light conversion body may be to emit provided light by converting the wavelength thereof. For example, the light control layer CCL may be a layer containing the quantum dot or a layer containing the phosphor.

The light control layer CCL may include a plurality of light control parts CCP1, CCP2, and CCP3. The light control parts CCP1, CCP2, and CCP3 may be spaced apart from each other.

Referring to FIG. 7, divided patterns BMP may be disposed between the light control parts CCP1, CCP2, and CCP3 which are spaced apart from each other, but embodiments of the present disclosure are not limited thereto. FIG. 7 illustrates that the divided patterns BMP do not overlap the light control parts CCP1, CCP2, and CCP3, but, in some embodiments, at least a portion of the edges of the light control parts CCP1, CCP2, and CCP3 may overlap the divided patterns BMP.

The light control layer CCL may include a first light control part CCP1 containing a first quantum dot QD1 which converts first color light provided from the light emitting device ED into second color light, a second light control part CCP2 containing a second quantum dot QD2 which converts the first color light into third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting device ED. For example, in some embodiments, the first quantum dot QD1 may be a red quantum dot to emit red light, and the second quantum dot QD2 may be a green quantum dot to emit green light. The same as described above related to quantum dots may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include(e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. In one or more embodiments, the scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 may respectively include base resins BR1, BR2, and BR3 in which the quantum dots QD1 and QD2 and the scatterer SP are dispersed. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may each independently be one or more of acrylic-based resins, urethane-based resins, silicone-based resins, epoxy-based resins, etc. The base resins BR1, BR2, and BR3 may be transparent resins. In some embodiments, the first base resin BR1, the second base resin BR2, and the third base resin BR3 may be the same as or different from each other.

In one or more embodiments, the light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce the light control parts CCP1, CCP2, and CCP3 from being exposed to moisture/oxygen. In some embodiments, the barrier layer BFL1 may cover the light control parts CCP1, CCP2, and CCP3. In some embodiments, a barrier layer BFL2 may be provided between the light control parts CCP1, CCP2, and CCP3 and the color filter layer CFL.

The barrier layers BFL1 and BFL2 may include at least one inorganic layer. For example, in some embodiments, the barrier layers BFL1 and BFL2 may each independently include an inorganic material. For example, the barrier layers BFL1 and BFL2 may each independently include one or more selected from a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In some embodiments, the barrier layers BFL1 and BFL2 may each further include an organic film. The barrier layers BFL1 and BFL2 may each be formed of a single layer or a plurality of layers.

In the display apparatus DD-a of one or more embodiments, the color filter layer CFL may be disposed on the light control layer CCL. For example, in some embodiments, the color filter layer CFL may be directly disposed on the light control layer CCL. In these embodiments, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, in some embodiments, the first filter CF1 may be a red filter, the second filter CF2 may be a green filter, and the third filter CF3 may be a blue filter. The filters CF1, CF2, and CF3 each may include a polymeric photosensitive resin and a pigment and/or dye. The first filter CF1 may include a red pigment and/or dye, the second filter CF2 may include a green pigment and/or dye, and the third filter CF3 may include a blue pigment and/or dye.

However, embodiments of the present disclosure are not limited thereto, and the third filter CF3 may not include (e.g., may exclude) any pigment and/or dye. The third filter CF3 may include a polymeric photosensitive resin and may not include(e.g., may exclude) any pigment and/or dye. The third filter CF3 may be transparent. The third filter CF3 may be formed of a transparent photosensitive resin.

In present disclosure, “not including a or any ‘component’” “excluding a or any ‘component’”, “‘component’-free”, and/or the like refers to that the “component” not being added, selected or utilized as a component in the composition, but the “component” of less than a suitable amount may still be included due to other impurities and/or external factors.

Furthermore, in one or more embodiments, the first filter CF1 and the second filter CF2 may be a yellow filter. The first filter CF1 and the second filter CF2 may not be separated but be provided as one filter.

In one or more embodiments, the color filter layer CFL may further include a light shielding part. The light shielding part may be a black matrix. The light shielding part may include an organic light shielding material and/or an inorganic light shielding material containing a black pigment and/or dye. The light shielding part may prevent or reduce light leakage, and may separate boundaries between the adjacent filters CF1, CF2, and CF3.

The first to third filters CF1, CF2, and CF3 may be disposed corresponding to the red light emitting region PXA-R, the green light emitting region PXA-G, and the blue light emitting region PXA-B, respectively.

A base substrate BL may be disposed on the color filter layer CFL. The base substrate BL may be a member which provides a base surface on which the color filter layer CFL, the light control layer CCL, and/or the like are disposed. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may not be provided.

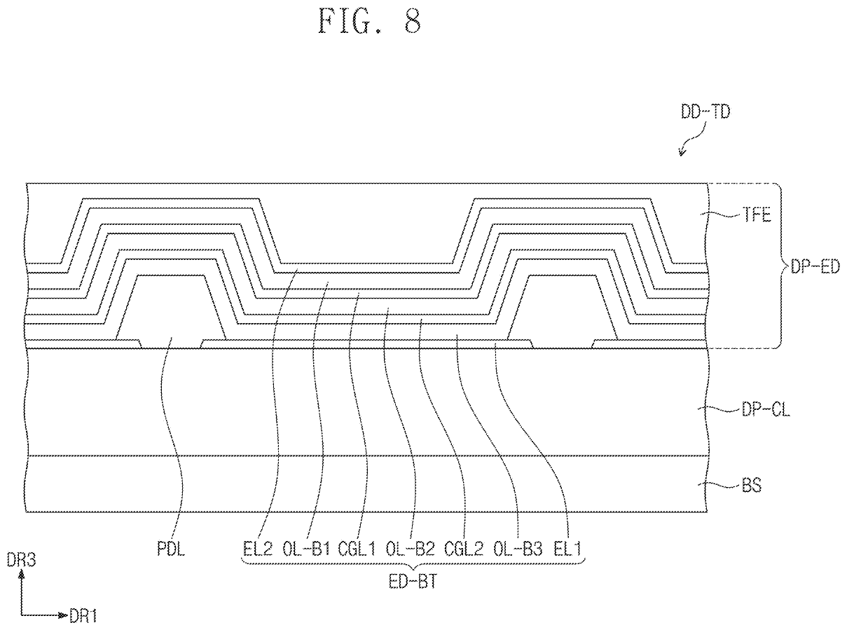

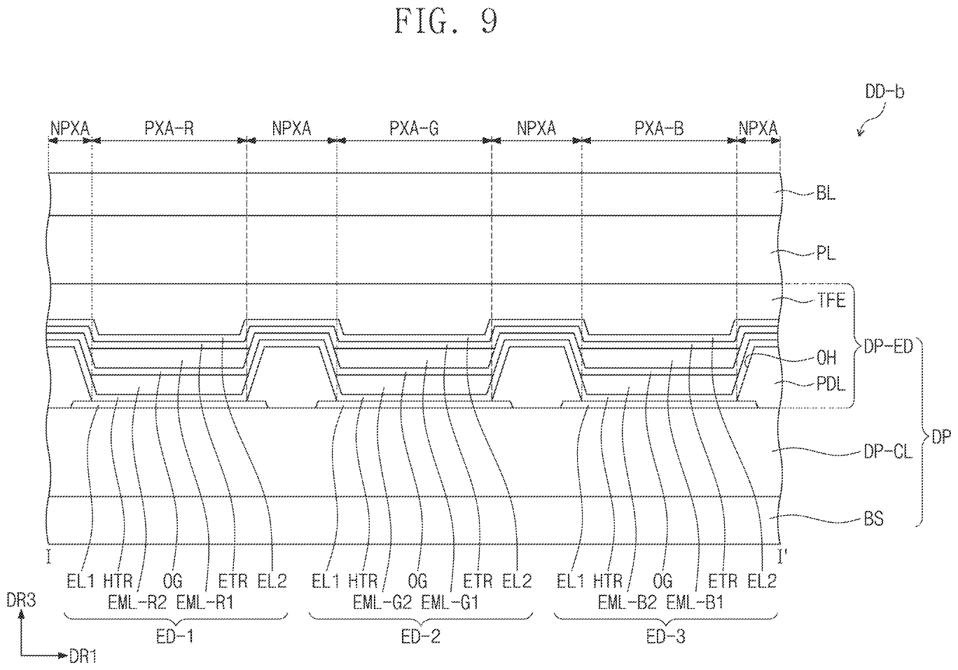

FIG. 8 is a cross-sectional view illustrating a portion of a display apparatus according to one or more embodiments. In the display apparatus DD-TD of one or more embodiments, a light emitting device ED-BT may include a plurality of light emitting structures OL-B1, OL-B2, and OL-B3. The light emitting device ED-BT may include a first electrode EL1 and a second electrode EL2 which face each other, and the plurality of light emitting structures OL-B1, OL-B2, and OL-B3 sequentially stacked in a thickness direction between the first electrode EL1 and the second electrode EL2. The light emitting structures OL-B1, OL-B2, and OL-B3 each may include an emission layer EML (FIG. 7) and a hole transport region HTR and an electron transport region ETR disposed with the emission layer EML (FIG. 7) therebetween.

For example, in some embodiments, the light emitting device ED-BT included in the display apparatus DD-TD of one or more embodiments may be a light emitting device having a tandem structure and including a plurality of emission layers.

In one or more embodiments illustrated in FIG. 8, all light beams respectively emitted from the light emitting structures OL-B1, OL-B2, and OL-B3 may be blue light. However, embodiments of the present disclosure are not limited thereto, and the light beams respectively emitted from the light emitting structures OL-B1, OL-B2, and OL-B3 may have wavelength ranges different from each other. For example, in some embodiments, the light emitting device ED-BT including the plurality of light emitting structures OL-B1, OL-B2, and OL-B3 which emit light beams having wavelength ranges different from each other may be to emit white light (e.g., combined white light).

Charge generation layers CGL1 and CGL2 may be respectively disposed between two of the neighboring light emitting structures OL-B1, OL-B2, and OL-B3. The charge generation layers CGL1 and CGL2 may include a p-type or kind charge (e.g., P-charge) generation layer and/or an n-type or kind charge (N-charge) generation layer. At least one selected from among the light emitting structures OL-B1, OL-B2, and OL-B3 included in the display device DD-TD of one or more embodiments may include the hetero compound of one or more embodiments described above. For example, at least one of the plurality of emission layers included in the light emitting element ED-BT may include the hetero compound of one or more embodiments.

Referring to FIG. 9, a display apparatus DD-b according to one or more embodiments may include light emitting devices ED-1, ED-2, and ED-3 in which two emission layers are stacked. Compared with the display apparatus DD illustrated in FIG. 2, the display apparatus DD-b illustrated in FIG. 9 has a difference in that first to third light emitting devices ED-1, ED-2, and ED-3 each include two emission layers stacked in a thickness direction. In each of the first to third light emitting devices ED-1, ED-2, and ED-3, the two emission layers may be to emit light in substantially the same wavelength region.

In one or more embodiments, the first light emitting device ED-1 may include a first red emission layer EML-R1 and a second red emission layer EML-R2. The second light emitting device ED-2 may include a first green emission layer EML-G1 and a second green emission layer EML-G2. In addition, the third light emitting device ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. In one or more embodiments, the emission auxiliary part OG may include an electron transport region, a charge generation layer, and a hole transport region that are sequentially stacked (e.g., in the stated order). The emission auxiliary part OG may be provided as a common layer in the whole of the first to third light emitting devices ED-1, ED-2, and ED-3. However, embodiments of the present disclosure are not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may each be disposed between the emission auxiliary part OG and the electron transport region ETR. The second red emission layer EML-R2, the second green emission layer EML-G2, and the second blue emission layer EML-B2 may each be disposed between the hole transport region HTR and the emission auxiliary part OG.

For example, in one or more embodiments, the first light emitting device ED-1 may include a first electrode EL1, a hole transport region HTR, a second red emission layer EML-R2, an emission auxiliary part OG, a first red emission layer EML-R₁, an electron transport region ETR, and a second electrode EL2 that are sequentially stacked (e.g., in the stated order). The second light emitting device ED-2 may include a first electrode EL1, a hole transport region HTR, a second green emission layer EML-G2, an emission auxiliary part OG, a first green emission layer EML-G1, an electron transport region ETR, and a second electrode EL2 that are sequentially stacked (e.g., in the stated order). The third light emitting device ED-3 may include a first electrode EL1, a hole transport region HTR, a second blue emission layer EML-B2, an emission auxiliary part OG, a first blue emission layer EML-B1, an electron transport region ETR, and a second electrode EL2 that are sequentially stacked (in the stated order).

In some embodiments, an optical auxiliary layer PL may be disposed on the display device layer DP-ED. The optical auxiliary layer PL may include a polarizing layer. The optical auxiliary layer PL may be disposed on the display panel DP and control reflected light in the display panel DP due to external light. In some embodiments, the optical auxiliary layer PL in the display apparatus may not be provided.

In one or more embodiments, at least one emission layer included in the display device DD-b in FIG. 9 may include the hetero compound of one or more embodiments described above. For example, in one or more embodiments, at least one of the first blue emission layer EML-B1 or the second blue emission layer EML-B2 may include the hetero compound of one or more embodiments.

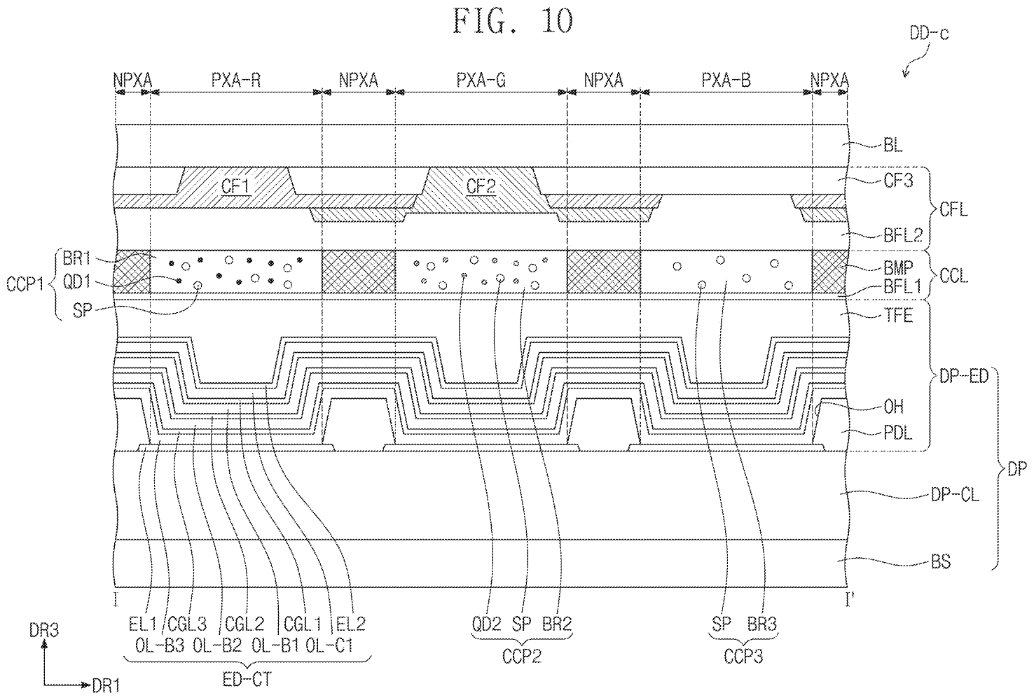

Unlike FIG. 8 and FIG. 9, FIG. 10 illustrates that a display apparatus DD-c may include four light emitting structures OL-B1, OL-B2, OL-B3, and OL-C1. A light emitting device ED-CT may include a first electrode EL1 and a second electrode EL2 which face each other, and first to fourth light emitting structures OL-B1, OL-B2, OL-B3, and OL-C1 that are stacked in a thickness direction between the first electrode EL1 and the second electrode EL2. Charge generation layers CGL1, CGL2, and CGL3 may be disposed between the first to fourth light emitting structures OL-B1, OL-B2, OL-B3, and OL-C1. Among the four light emitting structures, the first to third light emitting structures OL-B1, OL-B2, and OL-B3 may be to emit blue light, and the fourth light emitting structure OL-C1 may be to emit green light. However, embodiments of the present disclosure are not limited thereto, and the first to fourth light emitting structures OL-B1, OL-B2, OL-B3, and OL-C1 may be to emit light beams in different wavelength regions.

The charge generation layers CGL1, CGL2, and CGL3 disposed between adjacent light emitting structures OL-B1, OL-B2, OL-B3, and OL-C1 may include a p-type or kind charge (e.g., P-charge) generation layer and/or an n-type or kind charge (e.g., N-charge) generation layer.

At least one selected from among the light emitting structures OL-B1, OL-B2, OL-B3, and OL-C1 included in the display device DD-c according to one or more embodiments may include the hetero compound of one or more embodiments described above. For example, in one or more embodiments, at least one selected from among the first to third light emitting structures OL-B1, OL-B2, and OL-B3 may include the hetero compound of one or more embodiments described above.

The light emitting element/device ED described above according to one or more embodiments of the present disclosure may include the hetero compound of one or more embodiments in at least one functional layer disposed between the first electrode EL1 and the second electrode EL2, and may thus exhibit excellent or suitable light emitting efficiency and improved lifespan. For example, the hetero compound according to one or more embodiments may be included in the emission layer EML of the light emitting element ED of one or more embodiments, and the light emitting element of one or more embodiments may exhibit long lifespan.

In one or more embodiments, an electronic apparatus may include a display apparatus including a plurality of light emitting devices, and a control part which controls the display apparatus. The electronic apparatus of one or more embodiments may be a device that is activated according to an electrical signal. The electronic apparatus may include display apparatuses of one or more suitable embodiments. For example, the electronic apparatus may include not only large-sized electronic apparatuses such as a television set, a monitor, or an outdoor billboard but also include small- and medium-sized electronic apparatuses such as a personal computer, a laptop computer, a personal digital terminal, a display apparatus for a vehicle, a game console, a portable electronic device, or a camera.

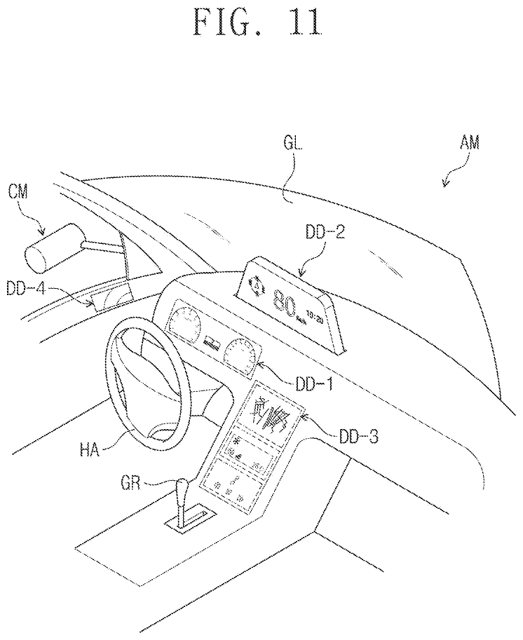

FIG. 11 is a view illustrating a vehicle AM in which first to fourth display apparatuses DD-1, DD-2, DD-3, and DD-4 are disposed. At least one selected from among the first to fourth display apparatuses DD-1, DD-2, DD-3, and DD-4 may include substantially the same configuration as the display apparatuses DD, DD-TD, DD-a, DD-b, and/or DD-c as described with reference to FIGS. 1, and 2, and 7 to 10.

FIG. 11 illustrates a vehicle AM, but this is a mere example. The first to fourth display apparatuses DD-1, DD-2, DD-3, and DD-4 may be disposed in another transportation apparatuses such as bicycles, motorcycles, trains, ships, and/or airplanes. In some embodiments, at least one selected from among the first to fourth display apparatuses DD-1, DD-2, DD-3, and DD-4 including substantially the same configuration as the display apparatuses DD, DD-TD, DD-a, DD-b, and/or DD-c of an embodiment may be employed in a personal computer, a laptop computer, a personal digital terminal, a game console, a portable electronic device, a television, a monitor, an outdoor billboard, and/or the like. These are merely provided as embodiments, and thus may be employed in other electronic apparatuses unless departing from the present disclosure.

In one or more embodiments, at least one selected from among the first to fourth display devices DD-1, DD-2, DD-3, and DD-4 may include the light emitting element ED of one or more embodiments described with reference to FIGS. 3 to 6.

The light emitting element ED of one or more embodiments may include the hetero compound of one or more embodiments. At least one selected from among the first to fourth display devices DD-1, DD-2, DD-3, and DD-4 may include the light emitting element ED including the hetero compound of one or more embodiments, and may thus have increased display lifespan.

Referring to FIG. 11, a vehicle AM may include a steering wheel HA and a gear GR for driving the vehicle AM. In addition, the vehicle AM may include a front window GL disposed so as to face a driver.

A first display apparatus DD-1 may be disposed in a first region overlapping the steering wheel HA. For example, the first display apparatus DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, etc. The first scale and the second scale may be indicated as a digital image.

A second display apparatus DD-2 may be disposed in a second region facing a driver seat and overlapping the front window GL. The driver seat may be a seat in which the steering wheel HA faces. For example, the second display apparatus DD-2 may be a head up display (HUD) which displays second information of the vehicle AM. The second display apparatus DD-2 may be optically transparent. The second information may include digital numbers which indicate a driving speed, and may further include information such as the current time. In some embodiments, the second information of the second display apparatus DD-2 may be projected to the front window GL to be displayed.

A third display apparatus DD-3 may be disposed in a third region adjacent to the gear GR. For example, the third display apparatus DD-3 may be disposed between the driver seat and a passenger seat and may be a center information display (CID) of vehicle for displaying third information. The passenger seat may be a seat spaced apart from the driver seat with the gear GR disposed therebetween. The third information may include information about traffic (e.g., navigation information), playing music or radio or a video (or an image), temperatures inside the vehicle AM, etc.

A fourth display apparatus DD-4 may be spaced apart from the steering wheel HA and the gear GR, and may be disposed in a fourth region adjacent to a side of the vehicle AM. For example, the fourth display apparatus DD-4 may be a digital side-view mirror which displays fourth information. The fourth display apparatus DD-4 may display an image outside the vehicle AM taken by a camera module CM disposed outside the vehicle AM. The fourth information may include an image outside the vehicle AM.

The above-described first to fourth information may be examples, and the first to fourth display apparatuses DD-1, DD-2, DD-3, and DD-4 may further display information about the inside and outside of the vehicle AM. The first to fourth information may include different information. However, embodiments of the present disclosure are not limited thereto, for example, in some embodiments, a part of the first to fourth information may include the same information as one another.

Hereinafter, with reference to Examples and Comparative Examples, a hetero compound according to one or more embodiments of the present disclosure and a light emitting element according to one or more embodiments will be specifically described. In addition, Examples are shown only for the understanding of the present disclosure, and the scope of the present disclosure is not limited thereto.

### Examples

### 1. Synthesis of Hetero Compounds

First, a process of synthesizing hetero compounds according to one or more embodiments of the present disclosure will be described in more detail by presenting a process of synthesizing each of Compounds E4, E5, E7, E8, E10, E12, E19, and E33 as an example. In addition, a process of synthesizing a hetero compound, which will be described hereinafter, is provided as an example, and thus the process of synthesizing a hetero compound according to one or more embodiments of the present disclosure is not limited to Examples.

### (1) Synthesis of Compound E4

### Synthesis of Intermediate E4-1

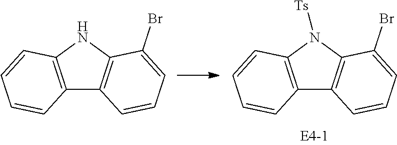 [SMILES: Brc1cccc2c1[nH]c1ccccc12 ; Cc1ccc(S(=O)(=O)n2c3ccccc3c3cccc(Br)c32)cc1]

1-bromo-9H-carbazole (CAS #=16807-11-7) (1 eq), 4-toluenesulfonyl chloride (TsCl) (1 eq), and KOH (1 eq)) were dissolved in acetone and refluxed overnight to obtain Intermediate E4-1. Intermediate E4-1 was determined through liquid chromatography-mass spectrometry (LC-MS), and the results are as follows.

C₁₉H₁₄BrNO₂S M+1: 400.3

### Synthesis of Intermediate E4-2

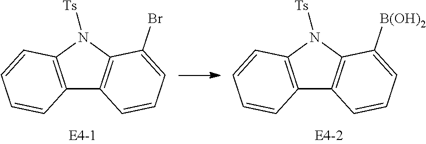 [SMILES: Cc1ccc(S(=O)(=O)n2c3ccccc3c3cccc(Br)c32)cc1 ; Cc1ccc(S(=O)(=O)n2c3ccccc3c3cccc(B(O)O)c32)cc1]

Intermediate E4-1 (1 eq) was dissolved in tetrahydrofuran (THF) and reacted with n-butyl lithium (1.2 eq) at −78° C., and after 1 hour, trimethyl borate (1.4 eq) was added dropwise. Thereafter, the temperature was slowly raised to room temperature to obtain Intermediate E4-2. Intermediate E4-2 was determined through LC-MS, and the results are as follows.

C₁₉H₁₆BNO₄S M+1: 365.21

### Synthesis of Intermediate E4-3

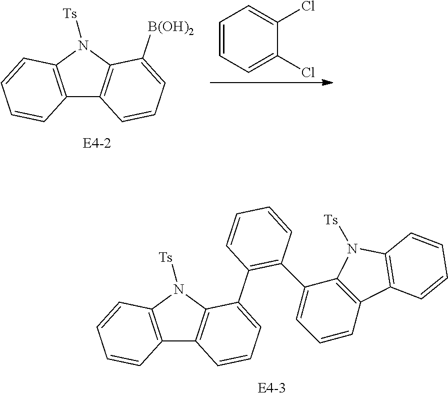 [SMILES: Cc1ccc(S(=O)(=O)n2c3ccccc3c3cccc(B(O)O)c32)cc1 ; Clc1ccccc1Cl ; Cc1ccc(S(=O)(=O)n2c3ccccc3c3cccc(-c4ccccc4-c4cccc5c6ccccc6n(S(=O)(=O)c6ccc(C)cc6)c45)c32)cc1]

Intermediate E4-2 (1 eq), 1,2-dichlorobenzene (CAS #=95-50-1) (1 eq), Pd(PPh₃)₂Cl₂ (0.01 eq), and Na₂CO₃ (2 eq) were reacted in a solution of THF:H₂O=4:1 at 70° C. to obtain Intermediate E4-3. Intermediate E4-3 was determined through LC-MS, and the results are as follows.

C₄₄H₃₂N₂O₄S₂ M+1: 716.87

### Synthesis of Intermediate E4-4

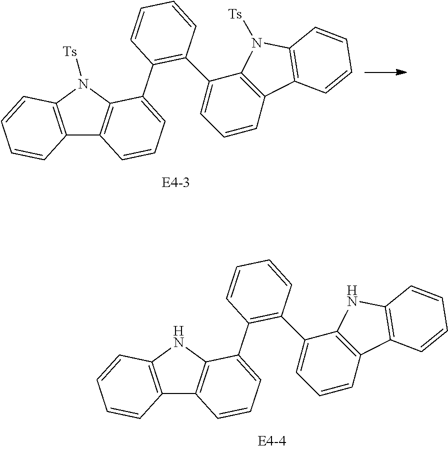 [SMILES: Cc1ccc(S(=O)(=O)n2c3ccccc3c3cccc(-c4ccccc4-c4cccc5c6ccccc6n(S(=O)(=O)c6ccc(C)cc6)c45)c32)cc1 ; c1ccc(-c2cccc3c2[nH]c2ccccc23)c(-c2cccc3c2[nH]c2ccccc23)c1]

Intermediate E4-3 (1 eq) and KOH (5 eq) were dissolved in a solution of THF:H₂O=1:1, and refluxed overnight to obtain Intermediate E4-4. Intermediate E4-4 was determined through LC-MS, and the results are as follows.

C₃₀H₂₀N₂ M+1: 408.50

### Synthesis of Intermediate E4-5

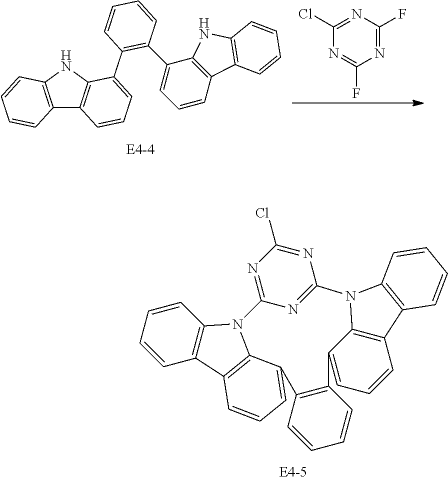

Intermediate E4-4 (2 eq) was dissolved in THF and reacted with n-butyl lithium at 0° C., and then 2-chloro-4,6-difluoro-1,3,5,-triazine was added dropwise. Thereafter, the mixture was stirred overnight at 70° C. to obtain Intermediate E4-5. Intermediate E4-5 was determined through LC-MS, and the results are as follows.

C₃₃H₁₈ClN₅ M+1: 519.99

### Synthesis of Intermediate E4-6

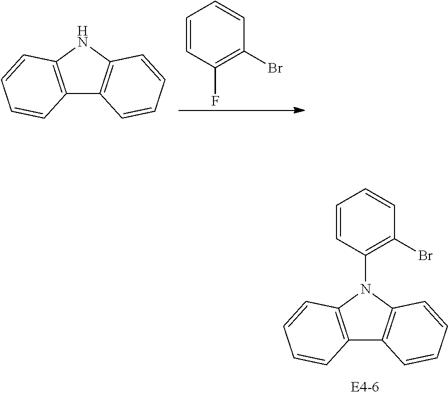

9H-carbazole (1 eq), 1-bromo-2-fluorobenzene (1.5 eq), and K₃PO₄ (2 eq) were dissolved in dimethylformamide (DMF) and stirred overnight at 160° C. Intermediate E4-6 was determined through LC-MS, and the results are as follows.

C₁₈H₁₂BrN M+1: 322.21

### Synthesis of Intermediate E4-7

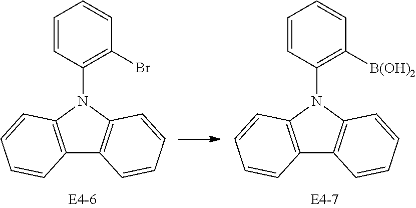 [SMILES: Brc1ccccc1-n1c2ccccc2c2ccccc21 ; OB(O)c1ccccc1-n1c2ccccc2c2ccccc21]

Intermediate E4-6 (1 eq) was dissolved in THF and reacted with n-butyl lithium (1.2 eq) at −78° C., and after 1 hour, trimethyl borate (1.4 eq) was added dropwise. Thereafter, the temperature was slowly raised to room temperature to obtain Intermediate E4-7. Intermediate E4-7 was determined through LC-MS, and the results are as follows.

C₁₈H₁₄BNO₂ M+1: 287.13

### Synthesis of Compound E4

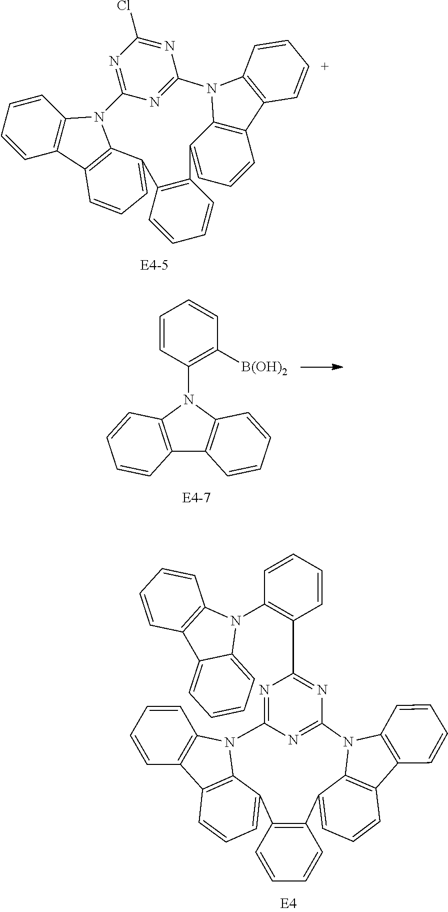 [SMILES: Clc1nc2nc(n1)n1c3ccccc3c3cccc(c4ccccc4c4cccc5c6c[c+]ccc6n2c45)c31 ; OB(O)c1ccccc1-n1c2ccccc2c2ccccc21 ; c1ccc(-n2c3ccccc3c3ccccc32)c(-c2nc3nc(n2)n2c4ccccc4c4cccc(c5ccccc5c5cccc6c7ccccc7n3c56)c42)c1]

Intermediate E4-5 (3.6 g), Intermediate E4-7 (2.8 g), tetrakis(triphenylphosphine)palladium (0.32 g), and potassium carbonate (2.4 g) were put into a reaction vessel and dissolved in 80 mL of toluene, 20 mL of ethanol, and 20 mL of distilled water, and then refluxed for 24 hours. After the reaction was completed, a reaction solution was extracted with ethyl acetate, a collected organic layer was dried over magnesium sulfate, and a residue obtained after evaporating a solvent was separated and purified through silica gel column chromatography to obtain 3.2 g (yield: 63%) of Compound E4. Compound E4 was determined through LC-MS, and the results are as follows.

C₅₁H₃₀N₆ M+1: 726.84

### (2) Synthesis of Compound E5

### Synthesis of Intermediate E5-1

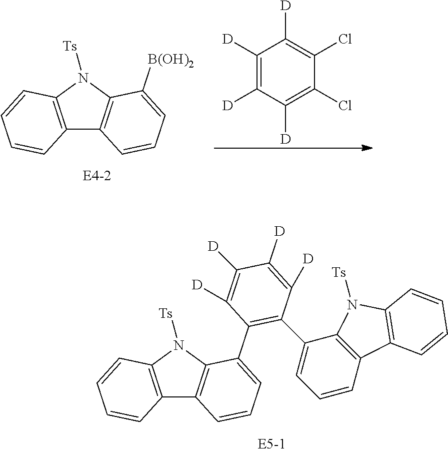 [SMILES: Cc1ccc(S(=O)(=O)n2c3ccccc3c3cccc(B(O)O)c32)cc1 ; [2H]c1c([2H])c([2H])c(Cl)c(Cl)c1[2H] ; [2H]c1c([2H])c([2H])c(-c2cccc3c4ccccc4n(S(=O)(=O)c4ccc(C)cc4)c23)c(-c2cccc3c4ccccc4n(S(=O)(=O)c4ccc(C)cc4)c23)c1[2H]]

Intermediate E4-2 (1 eq), 1,2-dichlorobenzene-d4 (CAS #=2199-69-1) (1 eq), Pd(PPh₃)₂Cl₂ (0.01 eq), and Na₂CO₃ (2 eq) were reacted in a solution of THF:H₂O=4:1 at 70° C. to obtain Intermediate E5-1. Intermediate E5-1 was determined through LC-MS, and the results are as follows.

C₄₄H₂₈D₄N₂O₄S₂ M+1: 720.89

### Synthesis of Intermediate E5-2

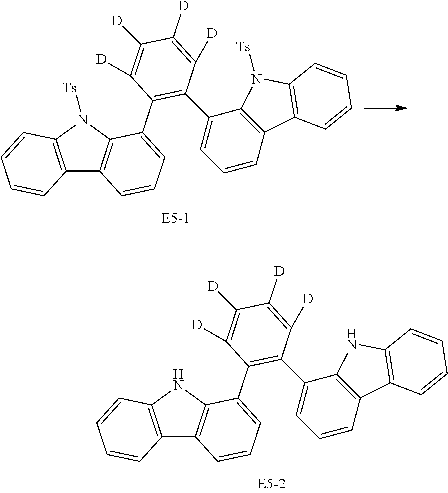 [SMILES: [2H]c1c([2H])c([2H])c(-c2cccc3c4ccccc4n(S(=O)(=O)c4ccc(C)cc4)c23)c(-c2cccc3c4ccccc4n(S(=O)(=O)c4ccc(C)cc4)c23)c1[2H] ; [2H]c1c([2H])c([2H])c(-c2cccc3c2[nH]c2ccccc23)c(-c2cccc3c2[nH]c2ccccc23)c1[2H]]

Intermediate E5-1 (1 eq) and KOH (5 eq) were dissolved in a solution of THF:H₂O=1:1, and refluxed overnight to obtain Intermediate E5-2. Intermediate E5-2 was determined through LC-MS, and the results are as follows.

C₃₀H₁₆D₄N₂ M+1: 412.53

### Synthesis of Intermediate E5-3

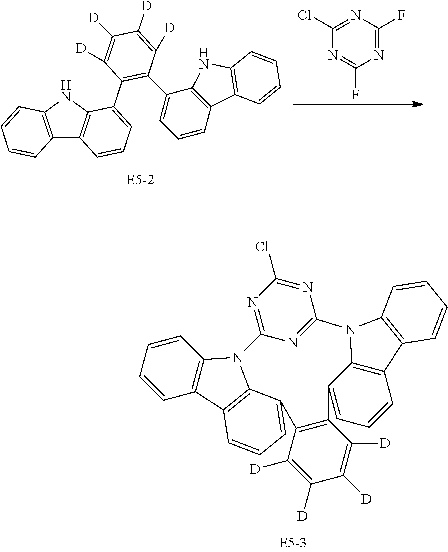 [SMILES: [2H]c1c([2H])c([2H])c(-c2cccc3c2[nH]c2ccccc23)c(-c2cccc3c2[nH]c2ccccc23)c1[2H] ; Fc1nc(F)nc(Cl)n1 ; [2H]c1c([2H])c([2H])c2c3cccc4c5ccccc5n(c5nc(Cl)nc(n5)n5c6ccccc6c6cccc(c2c1[2H])c65)c43]

Intermediate E5-2 (2 eq) was dissolved in THF and reacted with n-butyl lithium at 0° C., and then 2-chloro-4,6-difluoro-1,3,5-triazine was added dropwise. Thereafter, the mixture was stirred overnight at 70° C. to obtain Intermediate E5-3. Intermediate E5-3 was determined through LC-MS, and the results are as follows.

C₃₃H₁₄D₄ClN₅ M+1: 524.02

### Synthesis of Intermediate E5-4

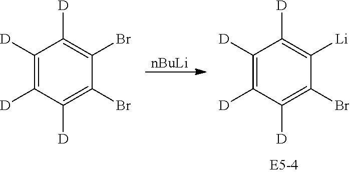

1,2-dibromobenzene-3,4,5,6-d4 (1 eq) was dissolved in THF, and Butyl lithium (1 eq) was slowly added dropwise at −78° C. to obtain Intermediate E5-4.

### Synthesis of Intermediate E5-5

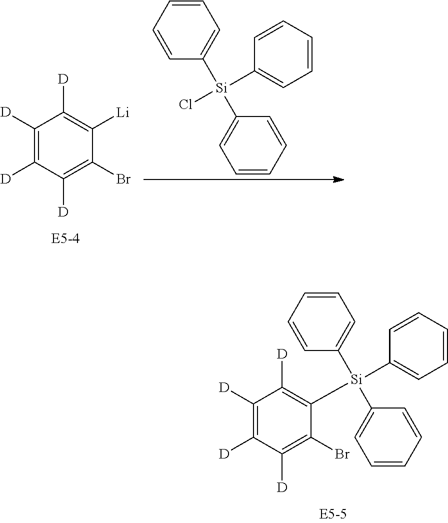

1,1′,1″-(chlorosilylidyne)tris[benzene] (3 eq) was dissolved in THF and added dropwise to a reaction solution of Intermediate E5-4. After slowly raising the temperature, the mixture was stirred overnight to obtain Intermediate E5-5. Intermediate E5-5 was determined through LC-MS, and the results are as follows.

C₂₄H₁₅D₄BrSi M+1: 419.43

### Synthesis of Intermediate E5-6

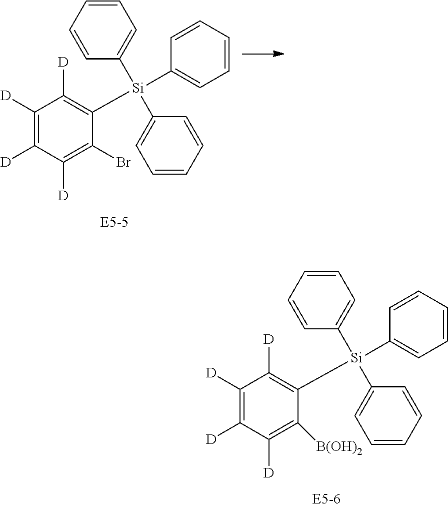 [SMILES: [2H]c1c([2H])c([2H])c([Si](c2ccccc2)(c2ccccc2)c2ccccc2)c(Br)c1[2H] ; [2H]c1c([2H])c([2H])c([Si](c2ccccc2)(c2ccccc2)c2ccccc2)c(B(O)O)c1[2H]]

Intermediate E5-5 (1 eq) was dissolved in THF and reacted with n-butyl lithium (1.2 eq) at −78° C., and after 1 hour, trimethyl borate was added dropwise. Thereafter, the temperature was slowly raised to room temperature to obtain Intermediate E5-6. Intermediate E5-6 was determined through LC-MS, and the results are as follows.

C₂₄H₁₇D₄BO₂Si M+1: 384.47

### Synthesis of Compound E5

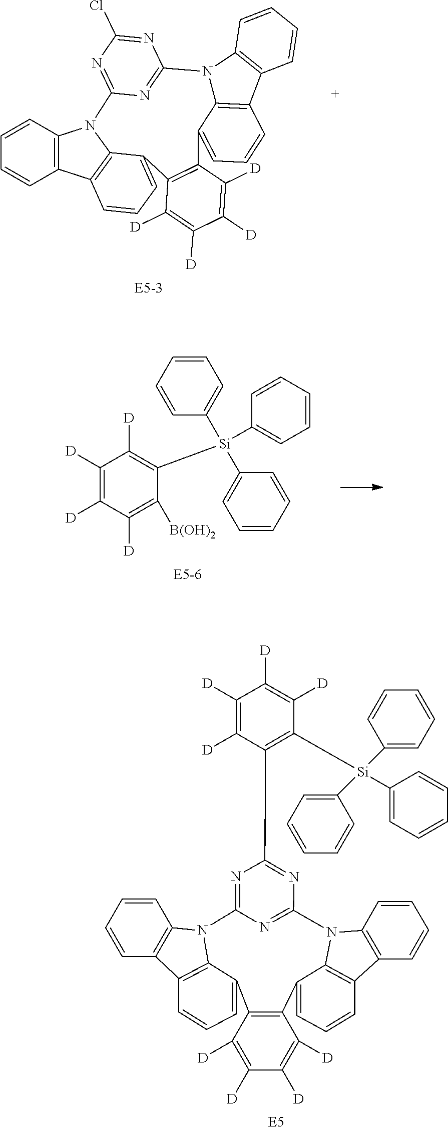 [SMILES: [2H]c1c([2H])c([2H])c2c3cccc4c5ccccc5n(c5nc(Cl)nc(n5)n5c6ccccc6c6cccc(c2c1[2H])c65)c43 ; [2H]c1c([2H])c([2H])c([Si](c2ccccc2)(c2ccccc2)c2ccccc2)c(B(O)O)c1[2H] ; [2H]c1c([2H])c([2H])c([Si](c2ccccc2)(c2ccccc2)c2ccccc2)c(-c2nc3nc(n2)n2c4ccccc4c4cccc(c5c([2H])c([2H])c([2H])c([2H])c5c5cccc6c7ccccc7n3c65)c42)c1[2H]]

Intermediate E5-3 (2.3 g), Intermediate E5-6 (2 g), tetrakis(triphenylphosphine)palladium (0.2 g), and potassium carbonate (1.5 g) were put into a reaction vessel and dissolved in 40 mL of toluene, 10 mL of ethanol, and 20 mL of distilled water, and then refluxed for 24 hours. After the reaction was completed, a reaction solution was extracted with ethyl acetate, a collected organic layer was dried over magnesium sulfate, and a residue obtained after evaporating a solvent was separated and purified through silica gel column chromatography to obtain 2.45 g (yield: 66%) of Compound E5. Compound E5 was determined through LC-MS, and the results are as follows.

C₅₇H₂₉D₈N₅Si M+1: 828.09

### (3) Synthesis of Compound E7

### Synthesis of Intermediate E7-1

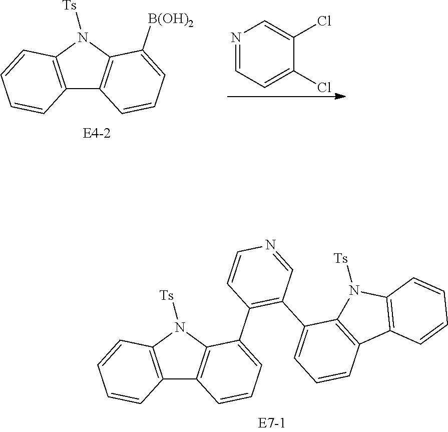 [SMILES: Cc1ccc(S(=O)(=O)n2c3ccccc3c3cccc(B(O)O)c32)cc1 ; Clc1ccncc1Cl ; Cc1ccc(S(=O)(=O)n2c3ccccc3c3cccc(-c4ccncc4-c4cccc5c6ccccc6n(S(=O)(=O)c6ccc(C)cc6)c45)c32)cc1]

Intermediate E4-2 (1 eq), 3,4-dichloropyridine (CAS #=55934-00-4) (1 eq), Pd(PPh₃)₂Cl₂ (0.01 eq), and Na₂CO₃ (2 eq) were reacted in a solution of THF:H₂O=4:1 at 70° C. to obtain Intermediate E7-1. Intermediate E7-1 was determined through LC-MS, and the results are as follows.

C₄₃H₃₁N₃O₄S₂ M+1: 717.86

### Synthesis of Intermediate E7-2

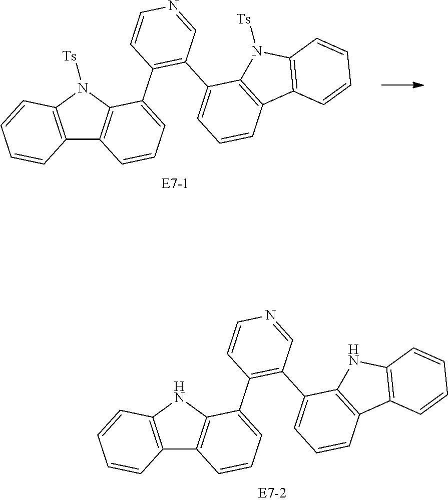 [SMILES: Cc1ccc(S(=O)(=O)n2c3ccccc3c3cccc(-c4ccncc4-c4cccc5c6ccccc6n(S(=O)(=O)c6ccc(C)cc6)c45)c32)cc1 ; c1ccc2c(c1)[nH]c1c(-c3ccncc3-c3cccc4c3[nH]c3ccccc34)cccc12]

Intermediate E7-1 (1 eq) and KOH (5 eq) were dissolved in a solution of THF:H₂O=1:1, and refluxed overnight to obtain Intermediate E7-2. Intermediate E7-2 was determined through LC-MS, and the results are as follows.

C₂₉H₁₁N₃ M+1: 409.49

### Synthesis of Intermediate E7-3

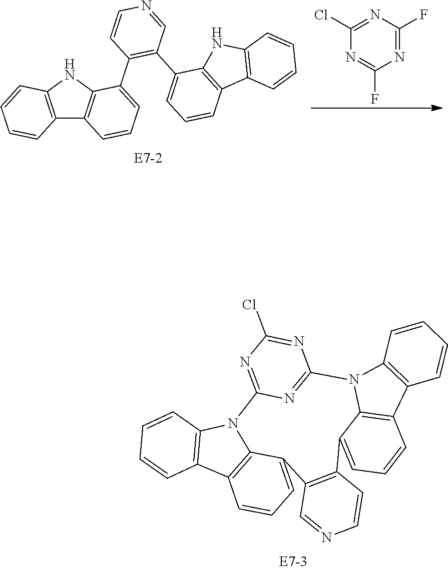 [SMILES: c1ccc2c(c1)[nH]c1c(-c3ccncc3-c3cccc4c3[nH]c3ccccc34)cccc12 ; Fc1nc(F)nc(Cl)n1 ; Clc1nc2nc(n1)n1c3ccccc3c3cccc(c4cnccc4c4cccc5c6ccccc6n2c45)c31]

Intermediate E7-2 (2 eq) was dissolved in THF and reacted with n-butyl lithium at 0° C., and then 2-chloro-4,6-difluoro-1,3,5,-triazine was added dropwise. Thereafter, the mixture was stirred overnight at 70° C. to obtain Intermediate E7-3. Intermediate E7-3 was determined through LC-MS, and the results are as follows.

C₃₂H₁₇ClN₆ M+1: 520.98

### Synthesis of Intermediate E7-4

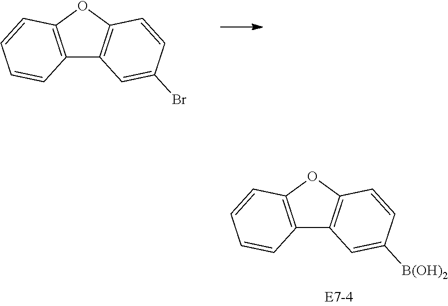 [SMILES: Brc1ccc2oc3ccccc3c2c1 ; OB(O)c1ccc2oc3ccccc3c2c1]

2-bromodibenzofuran (1 eq) was dissolved in THF and reacted with n-butyl lithium (1.2 eq) at −78° C., and after 1 hour, trimethyl borate was added dropwise. Thereafter, the temperature was slowly raised to room temperature to obtain Intermediate E7-4. Intermediate E7-4 was determined through LC-MS, and the results are as follows.

C₁₂H₉BO₃ M+1: 212.01

### Synthesis of Compound E7

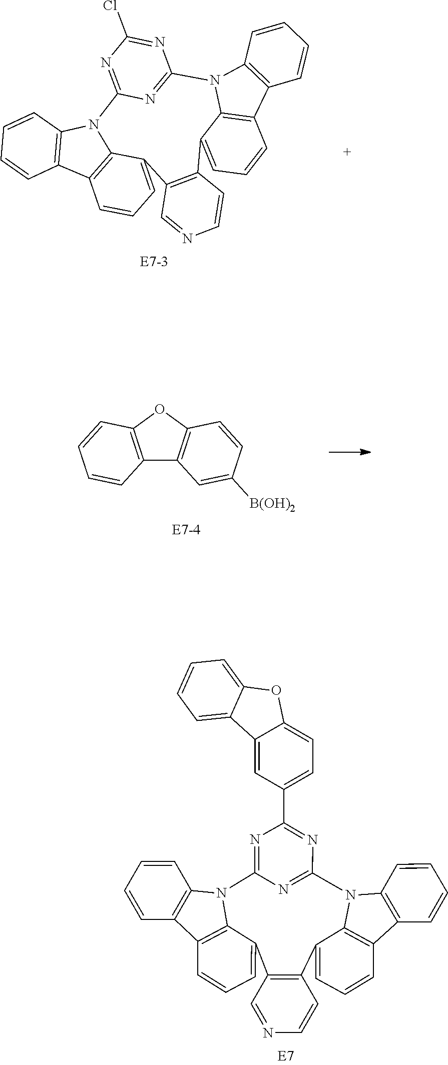 [SMILES: Clc1nc2nc(n1)n1c3ccccc3c3cccc(c4cnccc4c4cccc5c6ccccc6n2c45)c31 ; OB(O)c1ccc2oc3ccccc3c2c1 ; c1ccc2c(c1)oc1ccc(-c3nc4nc(n3)n3c5ccccc5c5cccc(c6cnccc6c6cccc7c8ccccc8n4c67)c53)cc12]

Intermediate E7-3 (3.1 g), Intermediate E7-4 (1.5 g), tetrakis(triphenylphosphine)palladium (0.28 g), and potassium carbonate (2.1 g) were put into a reaction vessel and dissolved in 80 mL of toluene, 20 mL of ethanol, and 20 mL of distilled water, and then refluxed for 24 hours. After the reaction was completed, a reaction solution was extracted with ethyl acetate, a collected organic layer was dried over magnesium sulfate, and a residue obtained after evaporating a solvent was separated and purified through silica gel column chromatography to obtain 2.88 g (yield: 51%) of Compound E7. Compound E7 was determined through LC-MS, and the results are as follows.

C₄₄H₂₄N₆O M+1: 828.09

### (4) Synthesis of Compound E8

### Synthesis of Intermediate E8-1

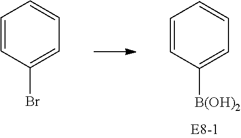 [SMILES: Brc1ccccc1 ; OB(O)c1ccccc1]

bromobenzene (1 eq) was dissolved in THF and reacted with n-butyl lithium (1.2 eq) at −78° C., and after 1 hour, trimethyl borate was added dropwise. Thereafter, the temperature was slowly raised to room temperature to obtain Intermediate E8-1. Intermediate E8-1 was determined through LC-MS, and the results are as follows.

C₆H₇B02 M+1: 121.93

### Synthesis of Compound E8

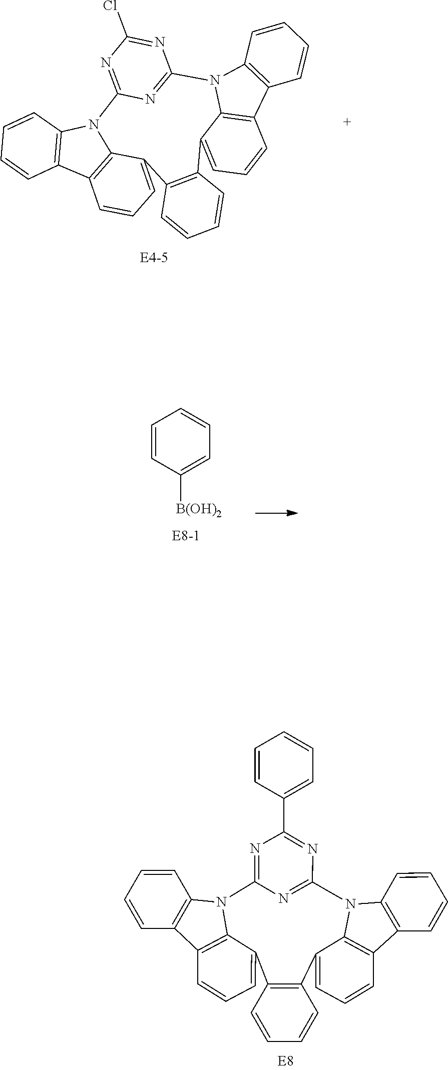 [SMILES: Clc1nc2nc(n1)n1c3ccccc3c3cccc(c4ccccc4c4cccc5c6ccccc6n2c45)c31 ; OB(O)c1ccccc1 ; c1ccc(-c2nc3nc(n2)n2c4ccccc4c4cccc(c5ccccc5c5cccc6c7ccccc7n3c56)c42)cc1]

Intermediate E4-5 (3.6 g), Intermediate E8-1 (1.0 g), tetrakis(triphenylphosphine)palladium (0.32 g), and potassium carbonate (2.4 g) were put into a reaction vessel and dissolved in 80 mL of toluene, 20 mL of ethanol, and 20 mL of distilled water, and then refluxed for 24 hours. After the reaction was completed, a reaction solution was extracted with ethyl acetate, a collected organic layer was dried over magnesium sulfate, and a residue obtained after evaporating a solvent was separated and purified through silica gel column chromatography to obtain 4.1 g (yield: 62%) of Compound E8. Compound E8 was determined through LC-MS, and the results are as follows.

C₃₉H₂₃N₅ M+1: 561.65

### (5) Synthesis of Compound E10

### Synthesis of Intermediate E10-1

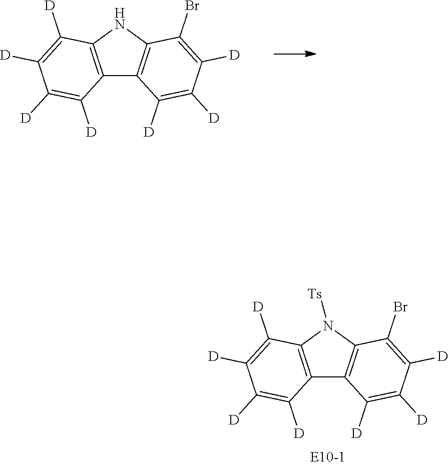 [SMILES: [2H]c1c([2H])c([2H])c2c([nH]c3c(Br)c([2H])c([2H])c([2H])c32)c1[2H] ; [2H]c1c([2H])c([2H])c2c(c1[2H])c1c([2H])c([2H])c([2H])c(Br)c1n2S(=O)(=O)c1ccc(C)cc1]

1-bromo-9H-carbazole-d7 (1 eq), TsCl (1 eq), and KOH(1 eq) were dissolved in acetone and refluxed overnight to obtain Intermediate E10-1. Intermediate E10-1 was determined through LC-MS, and the results are as follows.

C₁₉H₇D₇BrNO₂S M+1: 407.33

### Synthesis of Intermediate E10-2

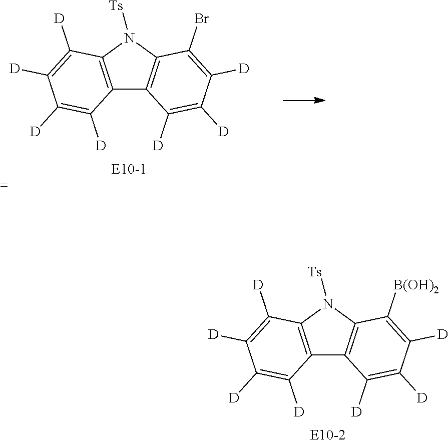 [SMILES: [2H]c1c([2H])c([2H])c2c(c1[2H])c1c([2H])c([2H])c([2H])c(Br)c1n2S(=O)(=O)c1ccc(C)cc1 ; [2H]c1c([2H])c([2H])c2c(c1[2H])c1c([2H])c([2H])c([2H])c(B(O)O)c1n2S(=O)(=O)c1ccc(C)cc1]

Intermediate E10-1 (1 eq) was dissolved in THF and reacted with n-butyl lithium (1.2 eq) at −78° C., and after 1 hour, trimethyl borate was added dropwise. Thereafter, the temperature was slowly raised to room temperature to obtain Intermediate E10-2. Intermediate E10-2 was determined through LC-MS, and the results are as follows.

C₁₉H₉D₇BNO₄S M+1: 372.25

### Synthesis of Intermediate E10-3

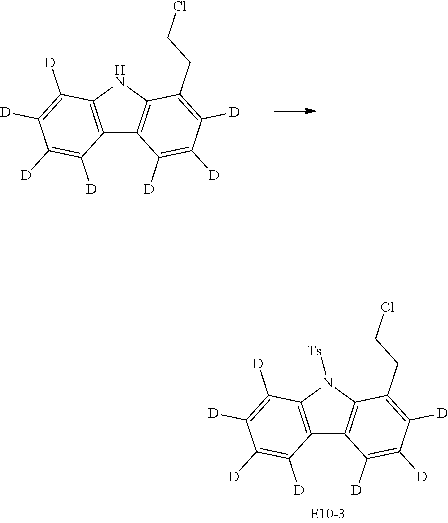 [SMILES: [2H]c1c([2H])c([2H])c2c([nH]c3c(CCCl)c([2H])c([2H])c([2H])c32)c1[2H] ; [2H]c1c([2H])c([2H])c2c(c1[2H])c1c([2H])c([2H])c([2H])c(CCCl)c1n2S(=O)(=O)c1ccc(C)cc1]

1-(2-chloroethyl)-9H-carbazole-2,3,4,5,6,7,8-d7 (1 eq), TsCl (1 eq), and KOH(1 eq) were dissolved in acetone and refluxed overnight to obtain Intermediate E10-3. Intermediate E10-3 was determined through LC-MS, and the results are as follows.

C₂₁H₁₁D₇ClNO₂S M+1: 390.93

### Synthesis of Intermediate E10-4

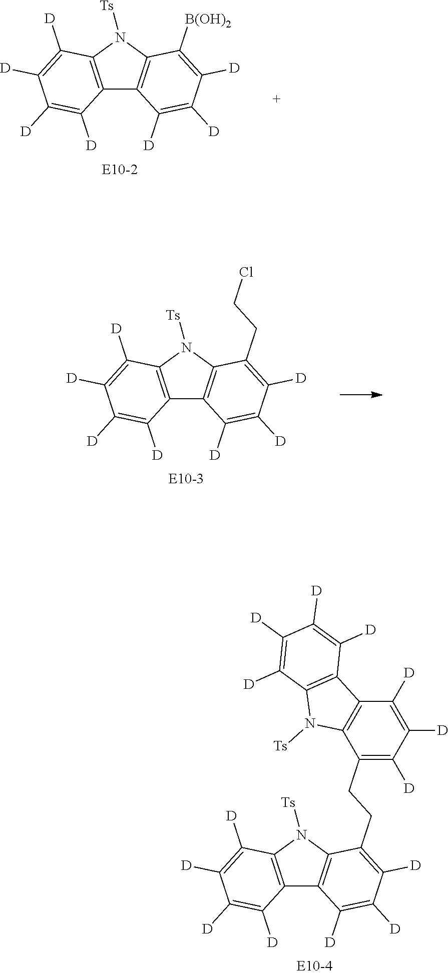 [SMILES: [2H]c1c([2H])c([2H])c2c(c1[2H])c1c([2H])c([2H])c([2H])c(B(O)O)c1n2S(=O)(=O)c1ccc(C)cc1 ; [2H]c1c([2H])c([2H])c2c(c1[2H])c1c([2H])c([2H])c([2H])c(CCCl)c1n2S(=O)(=O)c1ccc(C)cc1 ; [2H]c1c([2H])c([2H])c2c(c1[2H])c1c([2H])c([2H])c([2H])c(CCc3c([2H])c([2H])c([2H])c4c5c([2H])c([2H])c([2H])c([2H])c5n(S(=O)(=O)c5ccc(C)cc5)c34)c1n2S(=O)(=O)c1ccc(C)cc1]

Intermediate E10-2 (1 eq), Intermediate E10-3 (1 eq), Pd(PPh₃)₂Cl₂ (0.01 eq), and Na₂CO₃ (2 eq) were reacted in a solution of THF:H₂O=4:1 at 70° C. to obtain Intermediate E10-4. Intermediate E10-4 was determined through LC-MS, and the results are as follows.

C₄₀H₁₈D₁₄N₂O₄S₂ M+1: 682.91

### Synthesis of Intermediate E10-5

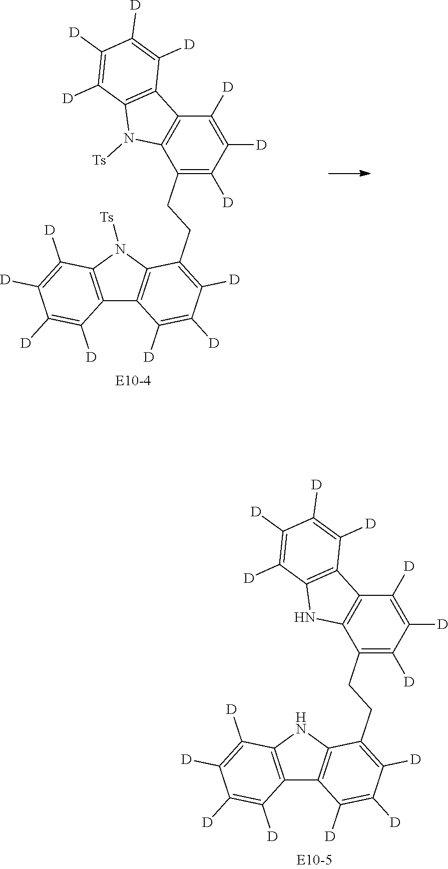 [SMILES: [2H]c1c([2H])c([2H])c2c(c1[2H])c1c([2H])c([2H])c([2H])c(CCc3c([2H])c([2H])c([2H])c4c5c([2H])c([2H])c([2H])c([2H])c5n(S(=O)(=O)c5ccc(C)cc5)c34)c1n2S(=O)(=O)c1ccc(C)cc1 ; [2H]c1c([2H])c([2H])c2c([nH]c3c(CCc4c([2H])c([2H])c([2H])c5c4[nH]c4c([2H])c([2H])c([2H])c([2H])c45)c([2H])c([2H])c([2H])c32)c1[2H]]

Intermediate E10-4 (1 eq) and KOH (5 eq) were dissolved in a solution of THF:H₂O=1:1, and refluxed overnight to obtain Intermediate E10-5. Intermediate E10-5 was determined through LC-MS, and the results are as follows.

C₂₆H₆D₁₄N₂ M+1: 374.55

### Synthesis of Intermediate E10-6

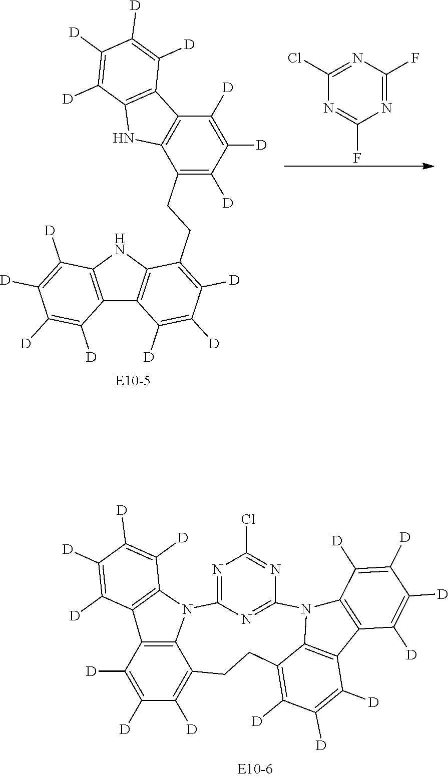 [SMILES: [2H]c1c([2H])c([2H])c2c([nH]c3c(CCc4c([2H])c([2H])c([2H])c5c4[nH]c4c([2H])c([2H])c([2H])c([2H])c45)c([2H])c([2H])c([2H])c32)c1[2H] ; Fc1nc(F)nc(Cl)n1 ; [2H]c1c([2H])c([2H])c2c(c1[2H])c1c([2H])c([2H])c([2H])c3c1n2-c1nc(Cl)nc(n1)-n1c2c([2H])c([2H])c([2H])c([2H])c2c2c([2H])c([2H])c([2H])c(c21)CC3]

Intermediate E10-5 (2 eq) was dissolved in THF and reacted with n-butyl lithium at 0° C., and then 2-chloro-4,6-difluoro-1,3,5,-triazine was added dropwise. Thereafter, the mixture was stirred overnight at 70° C. to obtain Intermediate E10-6. Intermediate E10-6 was determined through LC-MS, and the results are as follows.

C₂₉H₄D₁₄ClN₅ M+1: 486.03

### Synthesis of Intermediate E10-7

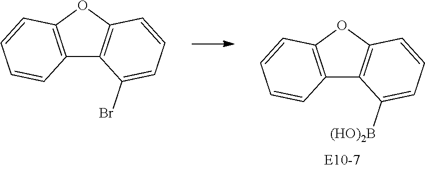 [SMILES: Brc1cccc2oc3ccccc3c12 ; OB(O)c1cccc2oc3ccccc3c12]

1-bromodibenzofuran (1 eq) was dissolved in THF and reacted with n-butyl lithium (1.2 eq) at −78° C., and after 1 hour, trimethyl borate was added dropwise. Thereafter, the temperature was slowly raised to room temperature to obtain Intermediate E10-7. Intermediate E10-7 was determined through LC-MS, and the results are as follows.

C₁₂H₉BO₃ M+1: 212.01

### Synthesis of Compound E10

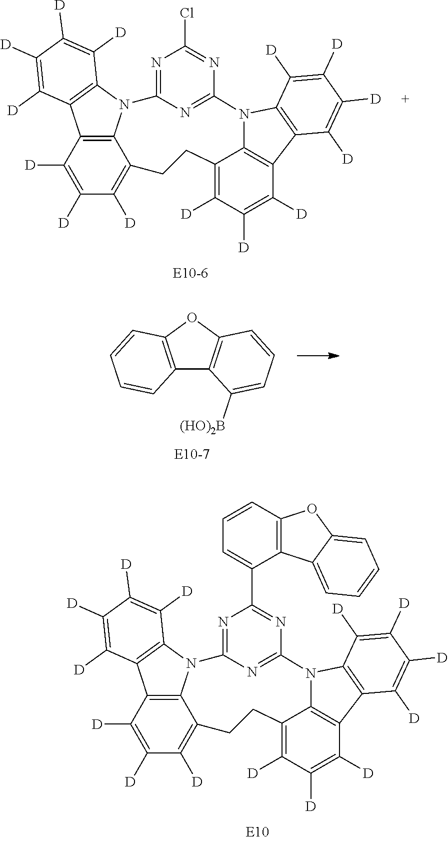 [SMILES: [2H]c1c([2H])c([2H])c2c(c1[2H])c1c([2H])c([2H])c([2H])c3c1n2-c1nc(Cl)nc(n1)-n1c2c([2H])c([2H])c([2H])c([2H])c2c2c([2H])c([2H])c([2H])c(c21)CC3 ; OB(O)c1cccc2oc3ccccc3c12 ; [2H]c1c([2H])c([2H])c2c(c1[2H])c1c([2H])c([2H])c([2H])c3c1n2-c1nc(-c2cccc4oc5ccccc5c24)nc(n1)-n1c2c([2H])c([2H])c([2H])c([2H])c2c2c([2H])c([2H])c([2H])c(c21)CC3]

Intermediate E10-6 (3.2 g), Intermediate E10-7 (1.7 g), tetrakis(triphenylphosphine)palladium (0.30 g), and potassium carbonate (2.3 g) were put into a reaction vessel and dissolved in 80 mL of toluene, 20 mL of ethanol, and 20 mL of distilled water, and then refluxed for 24 hours. After the reaction was completed, a reaction solution was extracted with ethyl acetate, a collected organic layer was dried over magnesium sulfate, and a residue obtained after evaporating a solvent was separated and purified through silica gel column chromatography to obtain 2.95 g (yield: 50%) of Compound E10. Compound E10 was determined through LC-MS, and the results are as follows.

C₄₁H₁₁D₁₄N₅O M+1: 617.77

### (6) Synthesis of Compound E12

### Synthesis of Intermediate E12-1

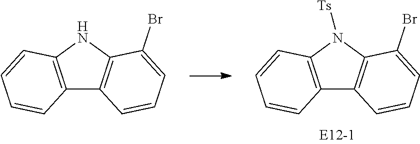 [SMILES: Brc1cccc2c1[nH]c1ccccc12 ; Cc1ccc(S(=O)(=O)n2c3ccccc3c3cccc(Br)c32)cc1]

1-bromo-9H-carbazole (CAS #=16807-11-7) (1 eq), TsCl (1 eq), and KOH (1 eq)) were dissolved in acetone and refluxed overnight to obtain Intermediate E12-1. Intermediate E12-1 was determined through LC-MS, and the results are as follows.

C₁₉H₁₄BrNO₂S M+1: 400.3

### Synthesis of Intermediate E12-2

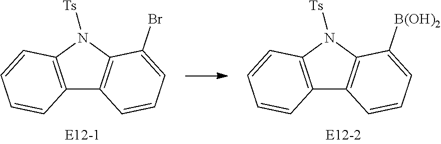 [SMILES: Cc1ccc(S(=O)(=O)n2c3ccccc3c3cccc(Br)c32)cc1 ; Cc1ccc(S(=O)(=O)n2c3ccccc3c3cccc(B(O)O)c32)cc1]

Intermediate E12-1 (1 eq) was dissolved in THF and reacted with n-butyl lithium (1.2 eq) at −78° C., and after 1 hour, trimethyl borate (1.4 eq) was added dropwise. Thereafter, the temperature was slowly raised to room temperature to obtain Intermediate E12-2. Intermediate E12-2 was determined through LC-MS, and the results are as follows.

C₁₉H₁₆BNO₄S M+1: 365.21

### Synthesis of Intermediate E12-3

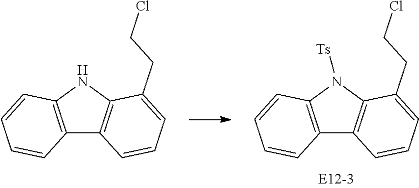 [SMILES: ClCCc1cccc2c1[nH]c1ccccc12 ; Cc1ccc(S(=O)(=O)n2c3ccccc3c3cccc(CCCl)c32)cc1]

1-(2-chloroethyl)-9H-carbazole (CAS #=855710-50-8) (1 eq), TsCl (1 eq), and KOH (1 eq)) were dissolved in acetone and refluxed overnight to obtain Intermediate E12-3. Intermediate E12-3 was determined through LC-MS, and the results are as follows.

C₂₁H₁₈ClNO₂S M+1: 390.93

### Synthesis of Intermediate E12-4

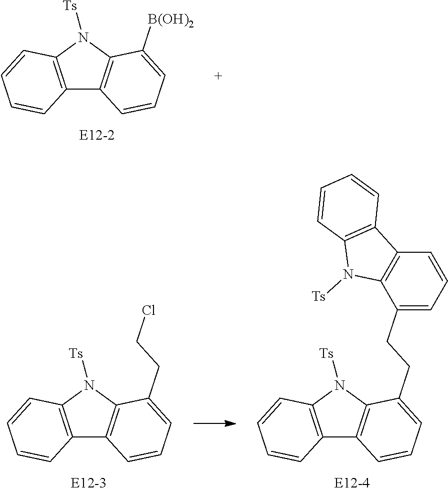 [SMILES: Cc1ccc(S(=O)(=O)n2c3ccccc3c3cccc(B(O)O)c32)cc1 ; Cc1ccc(S(=O)(=O)n2c3ccccc3c3cccc(CCCl)c32)cc1 ; Cc1ccc(S(=O)(=O)n2c3ccccc3c3cccc(CCc4cccc5c6ccccc6n(S(=O)(=O)c6ccc(C)cc6)c45)c32)cc1]

Intermediate E12-2 (1 eq), Intermediate E12-3 (1 eq), Pd(PPh₃)₂Cl₂ (0.01 eq), and Na₂CO₃ (2 eq) were reacted in a solution of THF:H₂O=4:1 at 70° C. to obtain Intermediate E12-4. Intermediate E12-4 was determined through LC-MS, and the results are as follows.

C₄₀H₃₂N₂O₄S₂ M+1: 668.83

### Synthesis of Intermediate E12-5

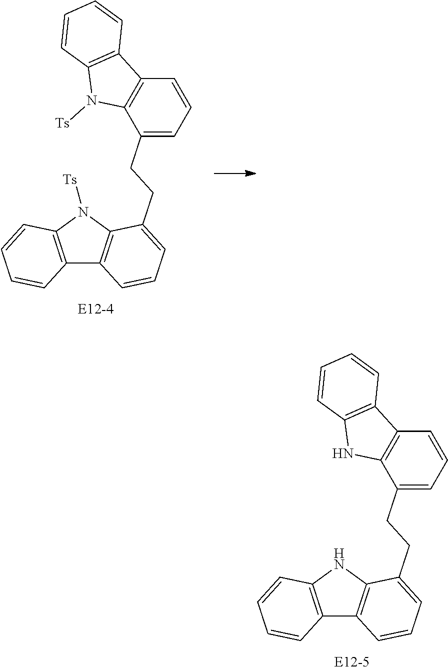 [SMILES: Cc1ccc(S(=O)(=O)n2c3ccccc3c3cccc(CCc4cccc5c6ccccc6n(S(=O)(=O)c6ccc(C)cc6)c45)c32)cc1 ; c1ccc2c(c1)[nH]c1c(CCc3cccc4c3[nH]c3ccccc34)cccc12]

Intermediate E12-4 (1 eq) and KOH (5 eq) were dissolved in a solution of THF:H₂O=1:1, and refluxed overnight to obtain Intermediate E12-5. Intermediate E12-5 was determined through LC-MS, and the results are as follows.

C₂₆H₂₀N₂ M+1: 360.46

### Synthesis of Intermediate E12-6

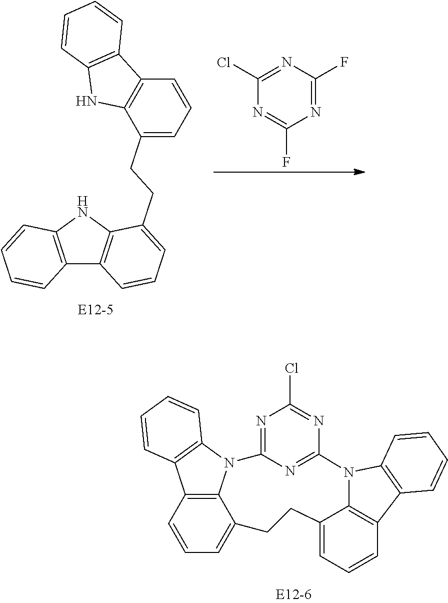 [SMILES: c1ccc2c(c1)[nH]c1c(CCc3cccc4c3[nH]c3ccccc34)cccc12 ; Fc1nc(F)nc(Cl)n1 ; Clc1nc2nc(n1)-n1c3ccccc3c3cccc(c31)CCc1cccc3c4ccccc4n-2c13]

Intermediate E12-5 (2 eq) was dissolved in THF and reacted with n-butyl lithium at 0° C., and then 2-chloro-4,6-difluoro-1,3,5,-triazine was added dropwise. Thereafter, the mixture was stirred overnight at 70° C. to obtain Intermediate E12-6. Intermediate E12-6 was determined through LC-MS, and the results are as follows.

C₂₉H₁ClN₅ M+1: 471.95

### Synthesis of Intermediate E12-7

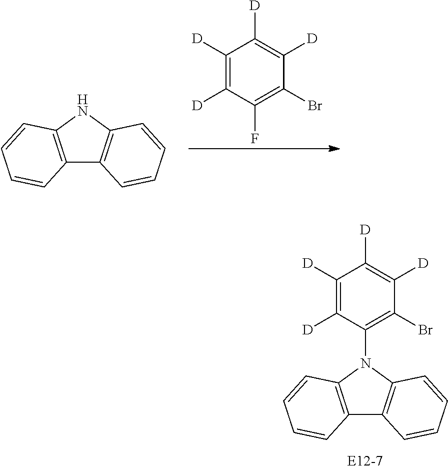 [SMILES: c1ccc2c(c1)[nH]c1ccccc12 ; [2H]c1c([2H])c([2H])c(Br)c(F)c1[2H] ; [2H]c1c([2H])c([2H])c(-n2c3ccccc3c3ccccc32)c(Br)c1[2H]]

9H-carbazole (5 eq), 5-bromo-6-fluoro-benzene-1,2,3,4-d4 (1.5 eq), and K₃PO₄ (2 eq) were dissolved in DMF and stirred overnight at 160° C. Intermediate E12-7 was determined through LC-MS, and the results are as follows.

C₁₈H₈D₄BrN M+1: 326.23

### Synthesis of Intermediate E12-8

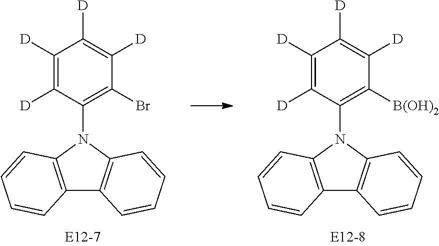 [SMILES: [2H]c1c([2H])c([2H])c(-n2c3ccccc3c3ccccc32)c(Br)c1[2H] ; [2H]c1c([2H])c([2H])c(-n2c3ccccc3c3ccccc32)c(B(O)O)c1[2H]]

Intermediate E12-7 (1 eq) was dissolved in THF and reacted with n-butyl lithium (1.2 eq) at −78° C., and after 1 hour, trimethyl borate was added dropwise. Thereafter, the temperature was slowly raised to room temperature to obtain Intermediate E12-8. Intermediate E12-8 was determined through LC-MS, and the results are as follows.

C₁₈H₁₀D₄BNO₂ M+1: 291.15

### Synthesis of Compound E12

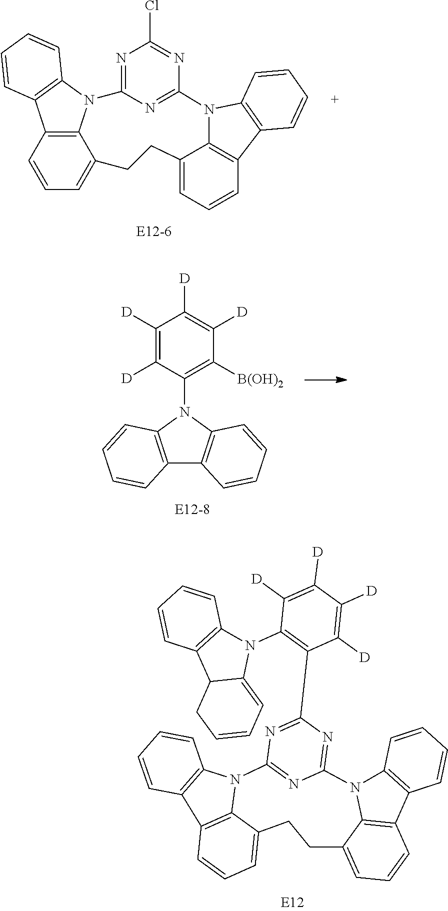 [SMILES: Clc1nc2nc(n1)-n1c3ccccc3c3cccc(c31)CCc1cccc3c4ccccc4n-2c13 ; [2H]c1c([2H])c([2H])c(-n2c3ccccc3c3ccccc32)c(B(O)O)c1[2H] ; [2H]c1c([2H])c([2H])c(N2C3=CC=CCC3c3ccccc32)c(-c2nc3nc(n2)-n2c4ccccc4c4cccc(c42)CCc2cccc4c5ccccc5n-3c24)c1[2H]]

Intermediate E12-6 (3.1 g), Intermediate E12-8 (2.3 g), tetrakis(triphenylphosphine)palladium (0.30 g), and potassium carbonate (2.3 g) were put into a reaction vessel and dissolved in 80 mL of toluene, 20 mL of ethanol, and 20 mL of distilled water, and then refluxed for 24 hours. After the reaction was completed, a reaction solution was extracted with ethyl acetate, a collected organic layer was dried over magnesium sulfate, and a residue obtained after evaporating a solvent was separated and purified through silica gel column chromatography to obtain 3.16 g (yield: 57%) of Compound E12. Compound E12 was determined through LC-MS, and the results are as follows.

C₄₇H₂₆D₄N₆ M+1: 682.82

### (7) Synthesis of Compound E19

### Synthesis of Compound E19

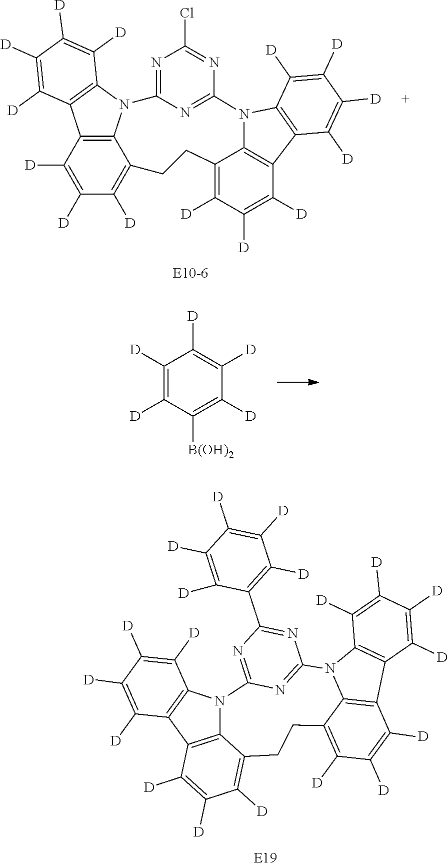 [SMILES: [2H]c1c([2H])c([2H])c2c(c1[2H])c1c([2H])c([2H])c([2H])c3c1n2-c1nc(Cl)nc(n1)-n1c2c([2H])c([2H])c([2H])c([2H])c2c2c([2H])c([2H])c([2H])c(c21)CC3 ; [2H]c1c([2H])c([2H])c(B(O)O)c([2H])c1[2H] ; [2H]c1c([2H])c([2H])c(-c2nc3nc(n2)-n2c4c([2H])c([2H])c([2H])c([2H])c4c4c([2H])c([2H])c([2H])c(c42)CCc2c([2H])c([2H])c([2H])c4c5c([2H])c([2H])c([2H])c([2H])c5n-3c24)c([2H])c1[2H]]

Intermediate E10-6 (3.1 g), 1-(phenyl-2,3,4,5,6-d5)boronic acid (CAS #=215527-70-1) (1 g), tetrakis(triphenylphosphine)palladium (0.30 g), and potassium carbonate (2.3 g) were put into a reaction vessel and dissolved in 80 mL of toluene, 20 mL of ethanol, and 20 mL of distilled water, and then refluxed for 24 hours. After the reaction was completed, a reaction solution was extracted with ethyl acetate, a collected organic layer was dried over magnesium sulfate, and a residue obtained after evaporating a solvent was separated and purified through silica gel column chromatography to obtain 2.94 g (yield: 53%) of Compound E19. Compound E19 was determined through LC-MS, and the results are as follows.

C₃₅H₄D₁₉N₅ M+1: 532.72

### (8) Synthesis of Compound E33

### Synthesis of Intermediate E33-1

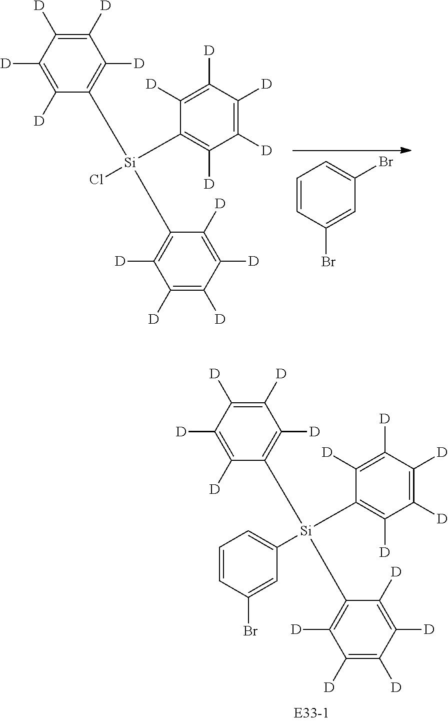

1,3-dibromobenzene (1 eq) was dissolved in THF and n-butyl lithium (1 eq) was slowly added dropwise at −78° C. A solution in which chlorotri(phenyl-d5)-silane (3 eq) was dissolved in THF was slowly added dropwise thereto. After slowly raising the temperature, the mixture was stirred overnight to obtain Intermediate E33-1. Intermediate E33-1 was determined through LC-MS, and the results are as follows.

C₂₄H₄D₁₅BrSi M+1: 430.5

### Synthesis of Intermediate E33-2

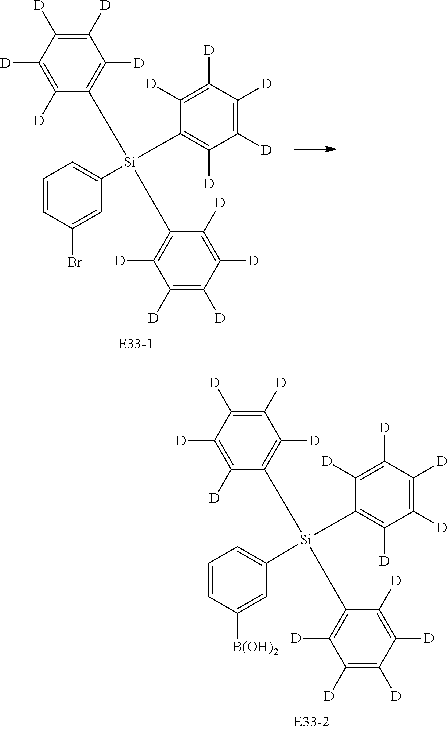 [SMILES: [2H]c1c([2H])c([2H])c([Si](c2cccc(Br)c2)(c2c([2H])c([2H])c([2H])c([2H])c2[2H])c2c([2H])c([2H])c([2H])c([2H])c2[2H])c([2H])c1[2H] ; [2H]c1c([2H])c([2H])c([Si](c2cccc(B(O)O)c2)(c2c([2H])c([2H])c([2H])c([2H])c2[2H])c2c([2H])c([2H])c([2H])c([2H])c2[2H])c([2H])c1[2H]]

Intermediate E33-1 (1 eq) was dissolved in THF and reacted with n-butyl lithium (1.2 eq) at −78° C., and after 1 hour, trimethyl borate was added dropwise. Thereafter, the temperature was slowly raised to room temperature to obtain Intermediate E33-2. Intermediate E33-2 was determined through LC-MS, and the results are as follows.

C₂₄H₆D₁₅BO₂Si M+1: 395.42

### Synthesis of Compound E33

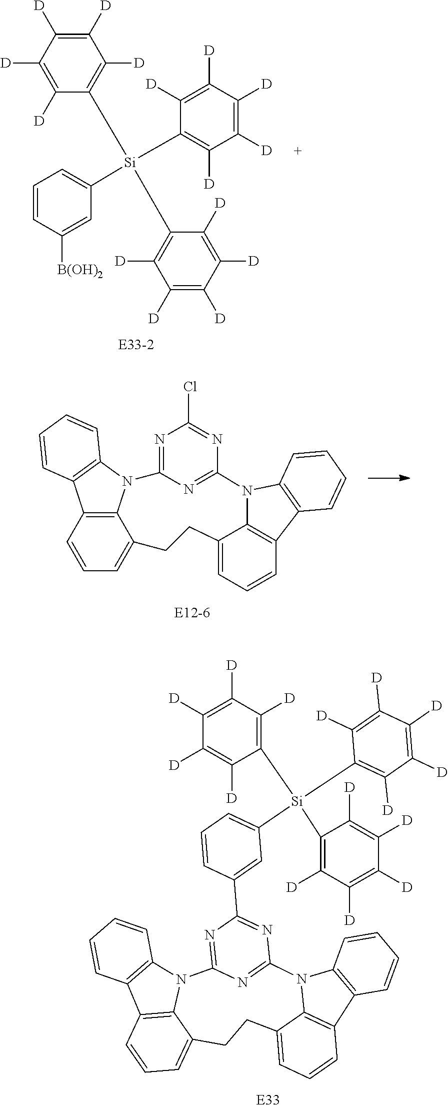 [SMILES: [2H]c1c([2H])c([2H])c([Si](c2cccc(B(O)O)c2)(c2c([2H])c([2H])c([2H])c([2H])c2[2H])c2c([2H])c([2H])c([2H])c([2H])c2[2H])c([2H])c1[2H] ; Clc1nc2nc(n1)-n1c3ccccc3c3cccc(c31)CCc1cccc3c4ccccc4n-2c13 ; [2H]c1c([2H])c([2H])c([Si](c2cccc(-c3nc4nc(n3)-n3c5ccccc5c5cccc(c53)CCc3cccc5c6ccccc6n-4c35)c2)(c2c([2H])c([2H])c([2H])c([2H])c2[2H])c2c([2H])c([2H])c([2H])c([2H])c2[2H])c([2H])c1[2H]]

Intermediate E12-6 (3.3 g), Intermediate E33-2 (3.3 g), tetrakis(triphenylphosphine)palladium (0.32 g), and potassium carbonate (2.4 g) were put into a reaction vessel and dissolved in 80 mL of toluene, 20 mL of ethanol, and 20 mL of distilled water, and then refluxed for 24 hours. After the reaction was completed, a reaction solution was extracted with ethyl acetate, a collected organic layer was dried over magnesium sulfate, and a residue obtained after evaporating a solvent was separated and purified through silica gel column chromatography to obtain 3.01 g (yield: 58%) of Compound E33. Compound E33 was determined through LC-MS, and the results are as follows.

C₅₃H₂₂D₁₅N₅Si M+1: 787.09

### 2. Preparation and Evaluation of Light Emitting Elements

Light emitting elements of an embodiment containing a hetero compound of an embodiment in an emission layer of the light emitting element were prepared utilizing a method described herein. Light emitting elements of Examples 1 to 8 were prepared utilizing hetero compounds of Compounds E4, E5, E7, E8, E10, E12, E19, and E33, respectively, which are Example Compounds described above, (each) as a host material of an emission layer. Comparative Examples 1 and 2 correspond to light emitting elements prepared utilizing Comparative Example Compounds X-1 and X-2, respectively, (each) as a dopant material of an emission layer.

### Example Compound

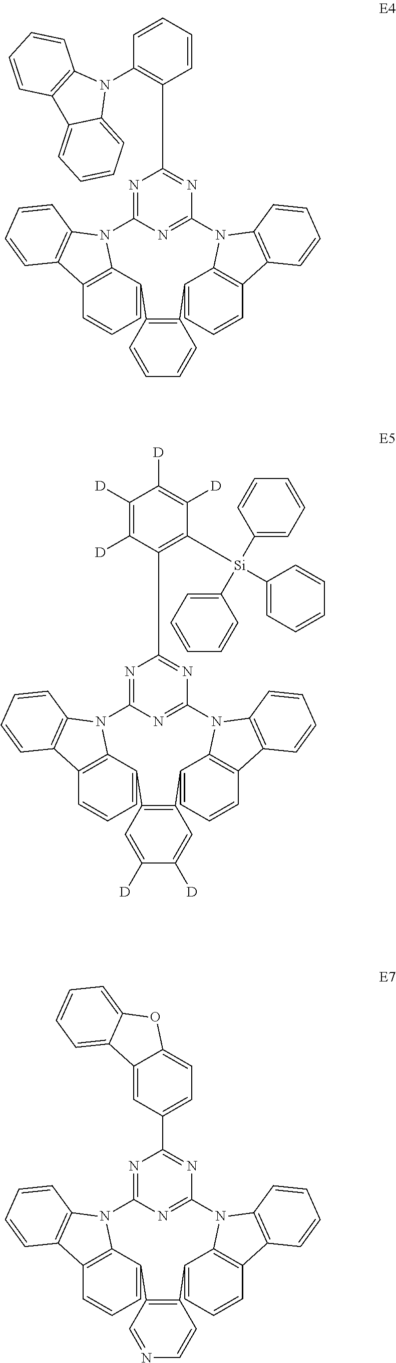 [SMILES: c1ccc(-n2c3ccccc3c3ccccc32)c(-c2nc3nc(n2)n2c4ccccc4c4cccc(c5ccccc5c5cccc6c7ccccc7n3c56)c42)c1 ; [2H]c1cc2c(cc1[2H])c1cccc3c4ccccc4n(c4nc(-c5c([2H])c([2H])c([2H])c([2H])c5[Si](c5ccccc5)(c5ccccc5)c5ccccc5)nc(n4)n4c5ccccc5c5cccc2c54)c13 ; c1ccc2c(c1)oc1ccc(-c3nc4nc(n3)n3c5ccccc5c5cccc(c6cnccc6c6cccc7c8ccccc8n4c67)c53)cc12]

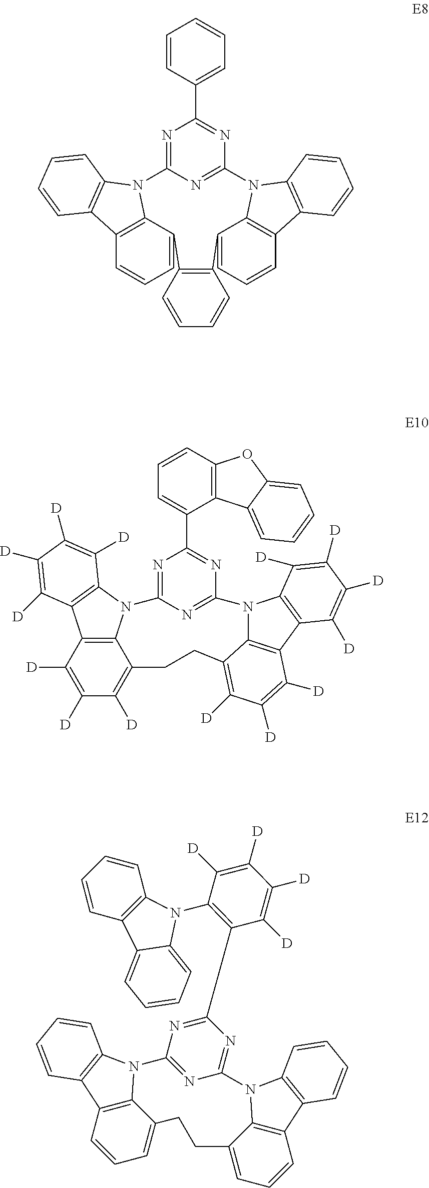 [SMILES: c1ccc(-c2nc3nc(n2)n2c4ccccc4c4cccc(c5ccccc5c5cccc6c7ccccc7n3c56)c42)cc1 ; [2H]c1c([2H])c([2H])c2c(c1[2H])c1c([2H])c([2H])c([2H])c3c1n2-c1nc(-c2cccc4oc5ccccc5c24)nc(n1)-n1c2c([2H])c([2H])c([2H])c([2H])c2c2c([2H])c([2H])c([2H])c(c21)CC3 ; [2H]c1c([2H])c([2H])c(-n2c3ccccc3c3ccccc32)c(-c2nc3nc(n2)-n2c4ccccc4c4cccc(c42)CCc2cccc4c5ccccc5n-3c24)c1[2H]]

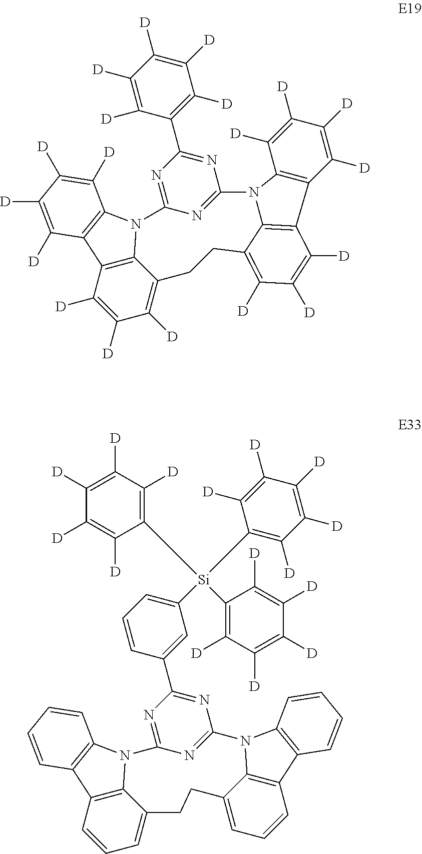 [SMILES: [2H]c1c([2H])c([2H])c(-c2nc3nc(n2)-n2c4c([2H])c([2H])c([2H])c([2H])c4c4c([2H])c([2H])c([2H])c(c42)CCc2c([2H])c([2H])c([2H])c4c5c([2H])c([2H])c([2H])c([2H])c5n-3c24)c([2H])c1[2H] ; [2H]c1c([2H])c([2H])c([Si](c2cccc(-c3nc4nc(n3)-n3c5ccccc5c5cccc(c53)CCc3cccc5c6ccccc6n-4c35)c2)(c2c([2H])c([2H])c([2H])c([2H])c2[2H])c2c([2H])c([2H])c([2H])c([2H])c2[2H])c([2H])c1[2H]]

### Comparative Example Compound

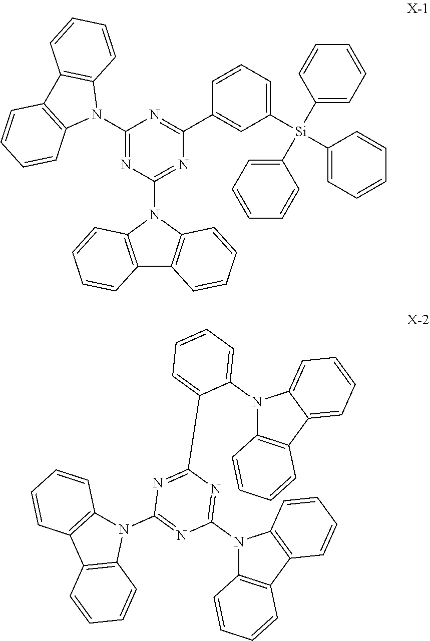

### Preparation of Light Emitting Elements

As for light emitting elements of Examples and Comparative Examples, as an anode, a glass substrate having an ITO electrode (Corning, 15 Ω/cm², 1200 Å) formed thereon was cut to a size of about 50 mm×50 mm×0.5 mm, subjected to ultrasonic cleaning with isopropyl alcohol and pure water for 5 minutes respectively and ultraviolet irradiation for 30 minutes, and then exposed to ozone for cleaning to be mounted on a vacuum deposition apparatus.

On the anode, a hole injection layer having a thickness of 100 Å was formed through deposition of HATCN, and on the hole injection layer, a hole transport layer having a thickness of 600 Å was formed through deposition of H-1-1 described above, and then on the hole transport layer, an auxiliary emission layer having a thickness of 50 Å was formed through deposition of HT33 described above.

Thereafter, Compound HT33 according to an embodiment described above, a corresponding Example compound or a corresponding Comparative Example compound, and Compound AD-41 described above were co-deposited in a weight ratio of 60: 27: 13 to form an emission layer having a thickness of 350 Å, and on the emission layer, a hole blocking layer having a thickness of 50 Å was formed through deposition of ETH2. Then, on the hole blocking layer, an electron transport layer having a thickness of 350 Å was formed through co-deposition of ETH2 and Liq in a ratio (e.g., amount) of 1:1, and then on the electron transport layer, an electron injection layer having a thickness of 15 Å was formed through deposition of LiF. Then, on the electron injection layer, a cathode having a thickness of 80 Å was formed through the deposition of Al to prepare a light emitting element. Each layer was formed through vacuum evaporation.

The compounds utilized in the preparation of each of the light emitting elements of Examples and Comparative Examples are disclosed below. The following materials were utilized for the preparation of the respective elements after sublimation-purifying commercially available products.

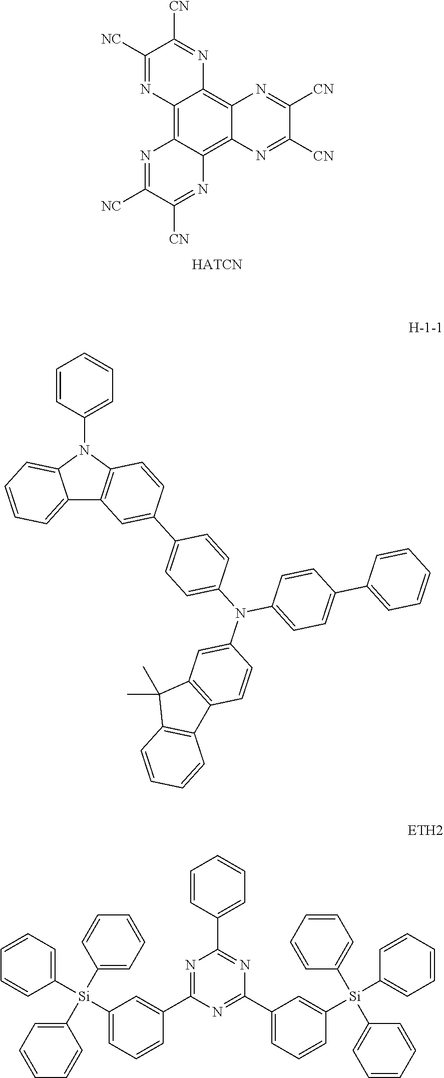

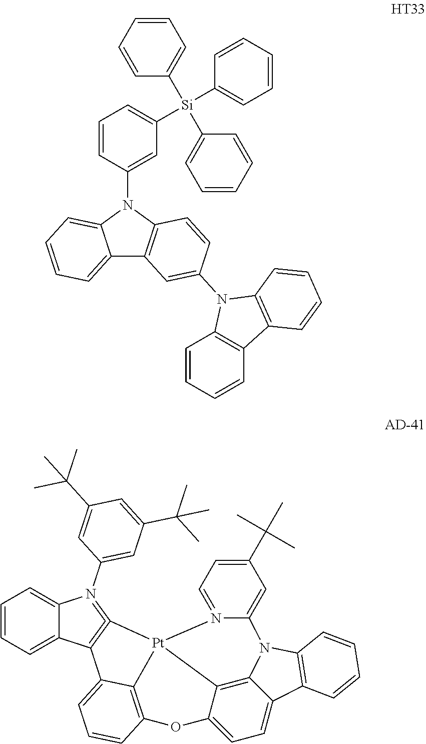

### Property Evaluation of Light Emitting Elements

Element efficiency and element lifespan of each of the light emitting elements prepared utilizing Example Compounds E4, E5, E7, E8, E10, E12, E19, and E33, and Comparative Example Compounds X-1 and X-2 were evaluated. Table 1 shows results of evaluation on each of the light emitting elements for Examples 1 to 8 and Comparative Examples 1 and 2. In order to evaluate the properties of the light emitting elements prepared in Examples 1 to 8 and Comparative Examples 1 and 2, driving voltage at a current density of 10 mA/cm², current density, and maximum quantum efficiency were measured. The driving voltage and current density of a light emitting element were determined utilizing a source meter (2400 series from Keithley Instrument), and the maximum quantum efficiency was determined utilizing an external quantum efficiency measuring apparatus (C9920-2-12 from Hamamatsu Photonics Co., Ltd.) In the evaluation of the maximum quantum efficiency, the luminance and current densities were measured utilizing a luminance meter that was calibrated for wavelength sensitivity, and the maximum quantum efficiency was converted under the assumption that an angular luminance distribution (Lambertian) was obtained with respect to a fully diffused reflective surface. The results of evaluating the properties of each of the light emitting elements are shown in Table 1.

TABLE 1

| | | | Maximum | Element | |
|---|---|---|---|---|---|
| | | Driving | quantum | relative | |
| | 1st compound | voltage | efficiency | lifespan | Emitted |
| | (Host material) | (V) | (%) | (%) | color |
| Example 1 | E8 | 4.7 | 24.2 | 102 | Blue |
| Example 2 | E10 | 4.8 | 23.4 | 111 | Blue |
| Example 3 | E5 | 5.4 | 24.1 | 101 | Blue |
| Example 4 | E19 | 5.1 | 23.8 | 121 | Blue |
| Example 5 | E12 | 5.4 | 24.7 | 112 | Blue |
| Example 6 | E33 | 5.2 | 23.9 | 119 | Blue |
| Example 7 | E4 | 5.3 | 23.8 | 101 | Blue |
| Example 8 | E7 | 4.9 | 24.3 | 108 | Blue |
| Comparative | Comparative | 5.5 | 21.8 | 100 | Blue |
| Example 1 | Example | | | | |
| | Compound X-1 | | | | |
| Comparative | Comparative | 5.7 | 20.2 | 97 | Blue |
| Example 2 | Example | | | | |
| | Compound X-2 | | | | |

Referring to the results of Table 1, it is seen that the light emitting elements of Examples utilizing the hetero compound according to one or more embodiments of the present disclosure (each) as a host material of an emission layer each had greater light emitting efficiency and lifespan than that of the light emitting elements of Comparative Examples. Example compounds (each) have a structure in which a hexagonal heterocycle containing 1 to 3 nitrogen atoms as ring-forming atoms is a core structure and two carbazole groups are connected to the hexagonal heterocycle, and also includes a linker structure connecting the two carbazole groups, thereby making a connection structure of the nitrogen-containing heterocycle and the two carbazole groups rigid to prevent or reduce vibronic motion of compound molecules in an excited state. Accordingly, Example compounds (each) have a relatively high triplet energy level, and when Example compounds are (each) applied to a light emitting element as a host material, high light emitting efficiency and long lifespan may be achieved. The light emitting element of one or more embodiments includes the first compound of one or more embodiments as a host material of a phosphorescent light emitting element, and may thus achieve high element efficiency, particularly in a blue light wavelength range.

It is seen that Comparative Example compound X-1 and Comparative Example compound X-2 included in Comparative Example 1 and Comparative Example 2, respectively, include a triazine core structure and a compound structure in which two carbazole groups are connected to the triazine core, but does not include a linker structure connecting the two carbazole groups, and thus have higher driving voltage, and lower maximum quantum efficiency and element service life than Example compounds when applied to a light emitting element.

A light emitting element of one or more embodiments of the present disclosure may exhibit improved element characteristics of high efficiency and long service life.

A hetero compound of one or more embodiments of the present disclosure is included in an emission layer of a light emitting element, and may thus contribute to relatively high efficiency and long service life of the light emitting element.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting element/device, the display device, the display apparatus, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the present disclosure has been described with reference to example embodiments of the present disclosure, it will be understood that the present disclosure should not be limited to these example embodiments but one or more suitable changes and modifications can be made by those skilled in the art without departing from the spirit and scope of the present disclosure.

Accordingly, the technical scope of the present disclosure is not intended to be limited to the content set forth in the detailed description of the present disclosure, but is intended to be defined by the appended claims and equivalents thereof.

What is claimed is:

1. A light emitting element comprising:
a first electrode;
a second electrode facing the first electrode; and
at least one functional layer between the first electrode and the second electrode,
wherein the at least one functional layer comprises a first compound represented by Formula 1: 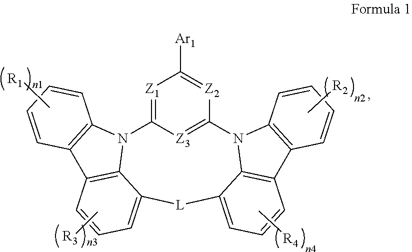
wherein in Formula 1,
Z₁ to Z₃ are each independently N or CR₅, and at least one selected from among Z₁ to Z₃ is N,
R₁ to R₅ are each independently hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Ar₁ is hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
L is a substituted or unsubstituted divalent oxy group, a substituted or unsubstituted divalent thio group, a substituted or unsubstituted divalent silyl group, a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms,
n1 and n2 are each independently an integer of 0 to 4, and
n3 and n4 are each independently an integer of 0 to 3.

2. The light emitting element of claim 1, wherein the at least one functional layer comprises:
a hole transport region on the first electrode;
an emission layer on the hole transport region; and
an electron transport region between the emission layer and the second electrode.

3. The light emitting element of claim 2, wherein the emission layer is to emit phosphorescence or delayed fluorescence, and comprises the first compound.

4. The light emitting element of claim 2, wherein the emission layer is to emit light having a central emission wavelength of about 430 nm to about 490 nm.

5. The light emitting element of claim 2, wherein the hole transport region comprises the first compound.

6. The light emitting element of claim 1, wherein L is represented by any one selected from among Formulas 2-1 to 2-4: 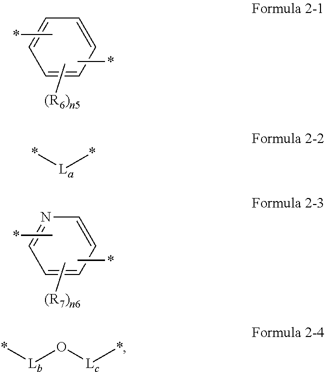
and
wherein in Formulas 2-1 to 2-4,
R₆ and R₇ are each independently hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Lₐ to L_{c} are each independently a substituted or unsubstituted alkylene group having 1 to 5 carbon atoms,
n5 is an integer of 0 to 4, and
n6 is an integer of 0 to 3.

7. The light emitting element of claim 1, wherein the first compound represented by Formula 1 is represented by Formula 1-1 or Formula 1-2: 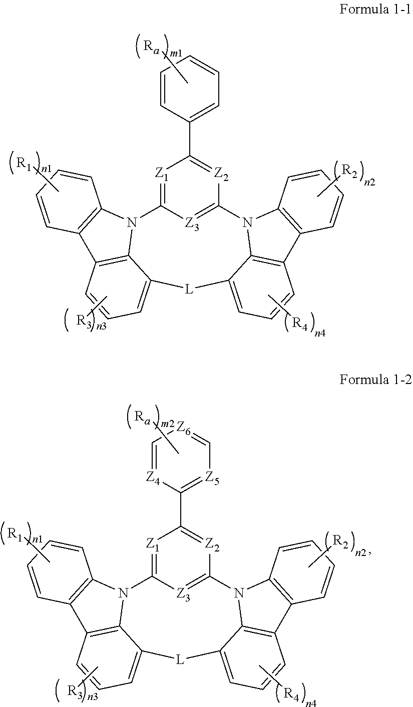
and
wherein in Formulas 1-1 and 1-2,
Z₄ to Z₆ are each independently N or CR_{c}, and at least one selected from among Z₄ to Z₆ is N,
Rₐ to Re are each independently hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring,
m1 is an integer of 0 to 5,
m2 is an integer of 0 to 2, and
Z₁ to Z₃, R₁ to R₄, n1 to n4, and L are the same as defined in Formula 1.

8. The light emitting element of claim 1, wherein Z₁ to Z₃ are all N.

9. The light emitting element of claim 1, wherein Ar₁ is represented by any one selected from among Formulas 3-1 to 3-5: 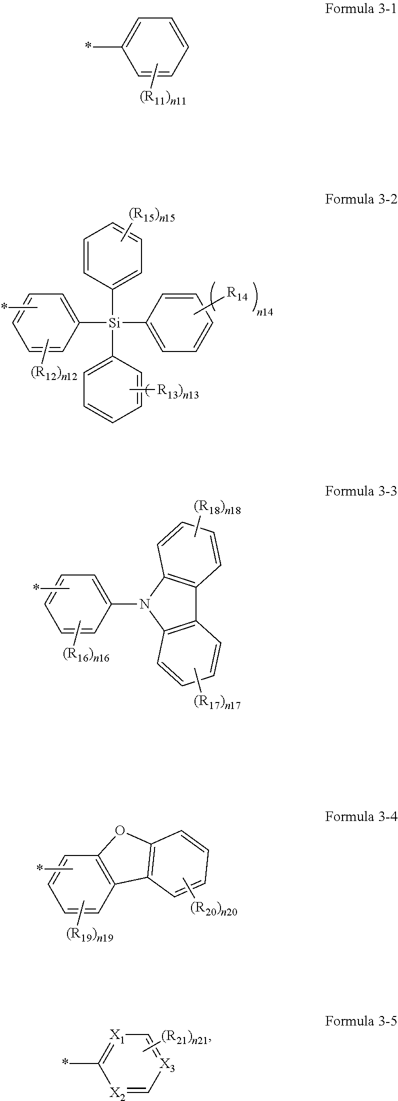
and
wherein in Formulas 3-1 to 3-5,
X₁ to X₃ are each independently N or CR₂₂, and at least one selected from among X₁ to X₃ is N,
R₁₁ to R₂₂ are each independently hydrogen, deuterium, a halogen, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
n11, and n13 to n15 are each independently an integer of 0 to 5,
n12, n16 to n18, and n20 are each independently an integer of 0 to 4,
n19 is an integer of 0 to 3, and
n21 is an integer of 0 to 2.

10. The light emitting element of claim 1, wherein R₁ to R₄ are each independently hydrogen or deuterium.

11. The light emitting element of claim 1, wherein L is represented by any one selected from among Formulas 2-5 to 2-8: 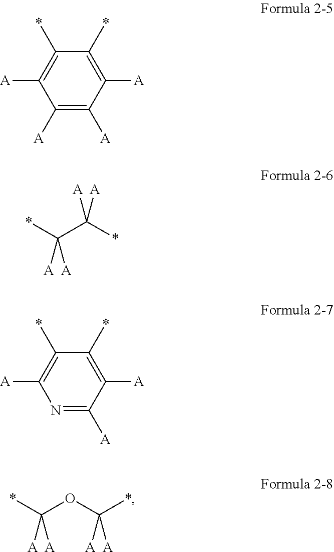
and
wherein in Formulas 2-5 to 2-8,
A is hydrogen or deuterium.

12. The light emitting element of claim 2, wherein the emission layer further comprises a second compound represented by Formula HT-1: 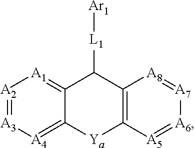
and
wherein in Formula HT-1,
A₁ to A₈ are each independently N or CR₅₁,
L₁ is a direct linkage, a substituted or unsubstituted arylene group having 6 to 30 ring-forming carbon atoms, or a substituted or unsubstituted heteroarylene group having 2 to 30 ring-forming carbon atoms,
Yₐ is direct linkage, CR₅₂R₅₃, or SiR₅₄R₅₅,
Ar₁ is a substituted or unsubstituted aryl group having 6 to 30 ring-forming carbon atoms, or a substituted or unsubstituted heteroaryl group having 2 to 30 ring-forming carbon atoms, and
R₅₁ to R₅₅ are each independently hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 ring-forming carbon atoms, a substituted or unsubstituted aryl group having 6 to 60 ring-forming carbon atoms, or a substituted or unsubstituted heteroaryl group having 2 to 60 ring-forming carbon atoms, and/or bonded to an adjacent group to form a ring.

13. The light emitting element of claim 2, wherein the emission layer further comprises a third compound represented by Formula D-1: 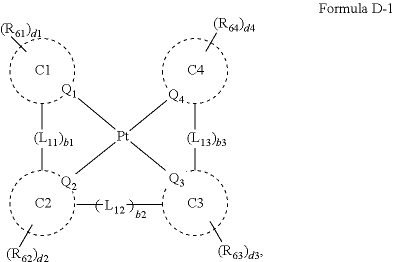
and
wherein in Formula D-1,
Q₁ to Q₄ are each independently C or N,
C1 to C4 are each independently a substituted or unsubstituted hydrocarbon ring having 5 to 30 ring-forming carbon atoms, or a substituted or unsubstituted heterocycle having 2 to 30 ring-forming carbon atoms,
L₁₁ to L₁₃ are each independently a direct linkage, 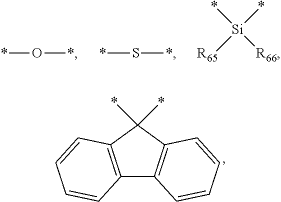
a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms,
b1 to b3 are each independently 0 or 1,
R₆₁ to R₆₆ are each independently hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 ring-forming carbon atoms, a substituted or unsubstituted aryl group having 6 to 60 ring-forming carbon atoms, or a substituted or unsubstituted heteroaryl group having 2 to 60 ring-forming carbon atoms, and/or bonded to an adjacent group to form a ring, and
d1 to d4 are each independently an integer of 0 to 4.

14. An electronic device, comprising at least one selected from among televisions, monitors, outdoor billboards, personal computers, laptop computers, personal digital terminals, vehicle display devices, game consoles, portable electronic devices, and cameras,
wherein the electronic device comprises at least one light emitting element comprising an emission layer, and
the emission layer comprises a first compound represented by Formula 1: 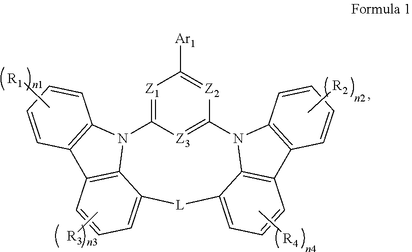
and
wherein in Formula 1,
Z₁ to Z₃ are each independently N or CR₅, and at least one selected from among Z₁ to Z₃ is N,
R₁ to R₅ are each independently hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Ar₁ is hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
L is a substituted or unsubstituted divalent oxy group, a substituted or unsubstituted divalent thio group, a substituted or unsubstituted divalent silyl group, a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms,
n1 and n2 are each independently an integer of 0 to 4, and
n3 and n4 are each independently an integer of 0 to 3.

15. A hetero compound represented by Formula 1: 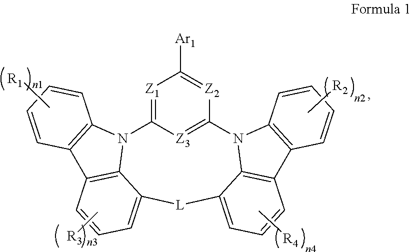
wherein in Formula 1,
Z₁ to Z₃ are each independently N or CR₅, and at least one selected from among Z₁ to Z₃ is N,
R₁ to R₅ are each independently hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Ar₁ is hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
L is a substituted or unsubstituted divalent oxy group, a substituted or unsubstituted divalent thio group, a substituted or unsubstituted divalent silyl group, a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms,
n1 and n2 are each independently an integer of 0 to 4, and
n3 and n4 are each independently an integer of 0 to 3.

16. The hetero compound of claim 15, wherein L is represented by any one selected from among Formulas 2-1 to 2-4: 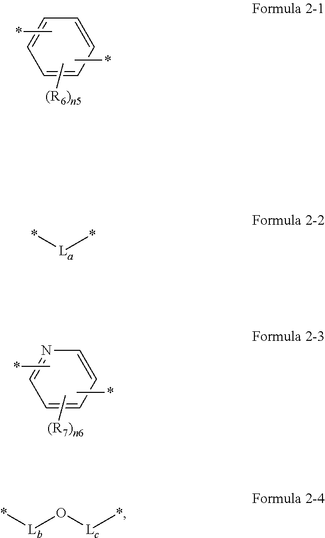
and
wherein in Formulas 2-1 to 2-4,
R₆ and R₇ are each independently hydrogen, deuterium, a halogen, a cyano group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Lₐ to L_{c} are each independently a substituted or unsubstituted alkylene group having 1 to 5 carbon atoms,
n5 is an integer of 0 to 4, and
n6 is an integer of 0 to 3.

17. The hetero compound of claim 15, wherein the hetero compound represented by Formula 1 is represented by Formula 1-1 or Formula 1-2: 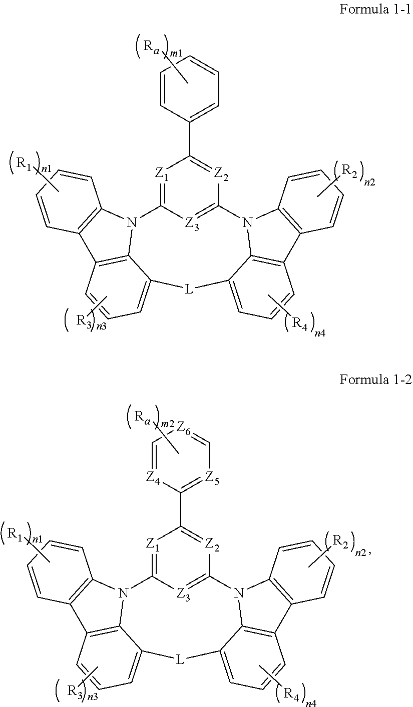
and
wherein in Formulas 1-1 and 1-2,
Z₄ to Z₆ are each independently N or CR_{c}, and at least one selected from among Z₄ to Z₆ is N,
Rₐ to Re are each independently hydrogen, deuterium, a halogen, a cyano group, a nitro group, a hydroxy group, a substituted or unsubstituted amine group, a substituted or unsubstituted oxy group, a substituted or unsubstituted thio group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring,
m1 is an integer of 0 to 5,
m2 is an integer of 0 to 2, and
Z₁ to Z₃, R₁ to R₄, n1 to n4, and L are the same as defined in Formula 1.

18. The hetero compound of claim 15, wherein Ar₁ is represented by any one selected from among Formulas 3-1 to 3-5: 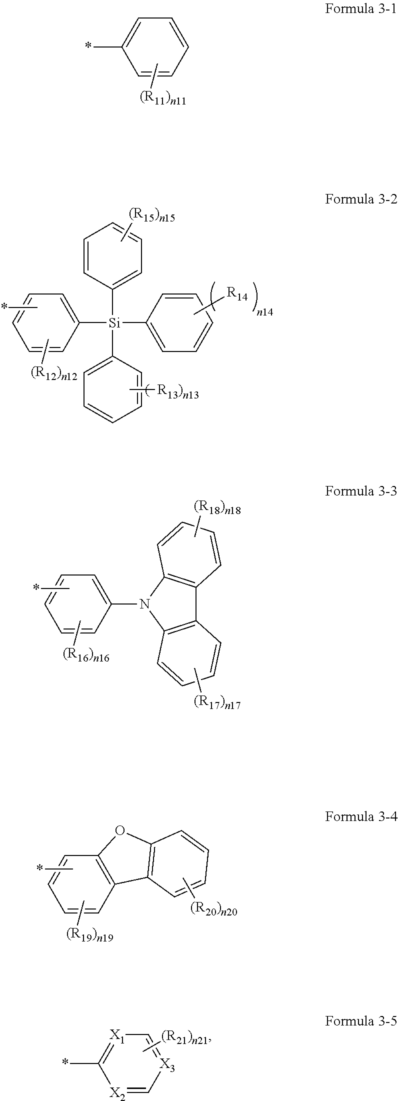
and
wherein in Formulas 3-1 to 3-5,
R₁₁ to R₂₁ are each independently hydrogen, deuterium, a halogen, a cyano group, a substituted or unsubstituted silyl group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring,
n11, and n13 to n15 are each independently an integer of 0 to 5,
n12, n16 to n18, and n20 are each independently an integer of 0 to 4,
n19 is an integer of 0 to 3, and
n21 is an integer of 0 to 2.

19. The hetero compound of claim 15, wherein L is represented by any one selected from among Formulas 2-5 to 2-8: 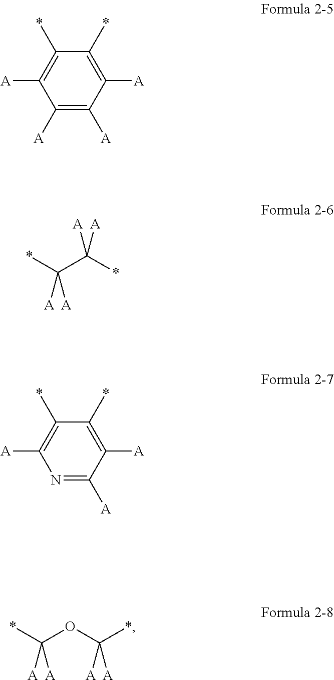
and
wherein in Formulas 2-5 to 2-8,
A is hydrogen or deuterium.

20. The hetero compound of claim 15, wherein the hetero compound comprises at least one selected from among compounds of Compound Group 1: 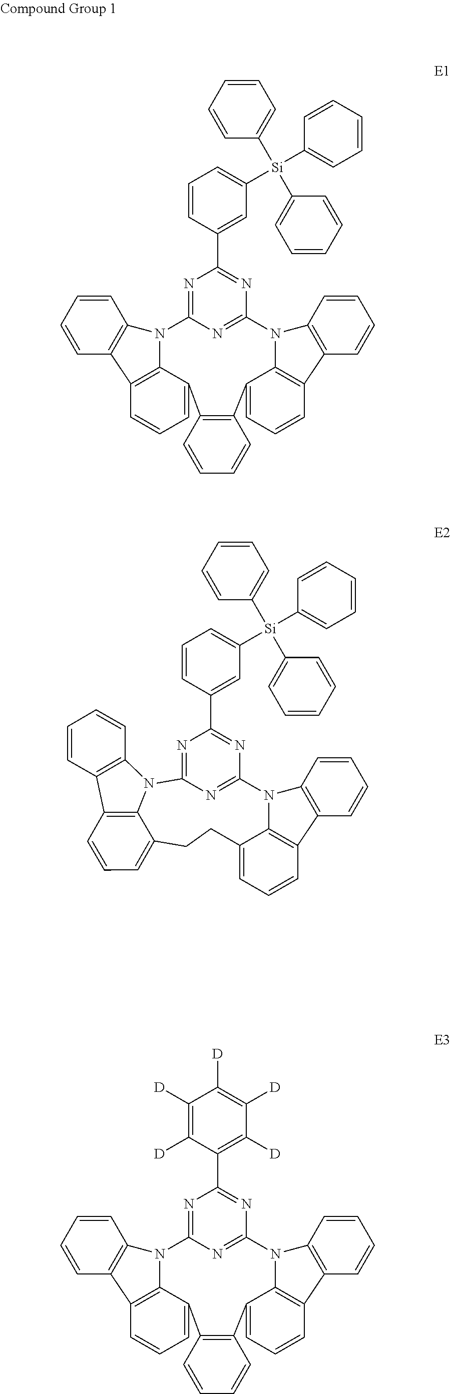 [SMILES: c1ccc([Si](c2ccccc2)(c2ccccc2)c2cccc(-c3nc4nc(n3)n3c5ccccc5c5cccc(c6ccccc6c6cccc7c8ccccc8n4c67)c53)c2)cc1 ; c1ccc([Si](c2ccccc2)(c2ccccc2)c2cccc(-c3nc4nc(n3)-n3c5ccccc5c5cccc(c53)CCc3cccc5c6ccccc6n-4c35)c2)cc1 ; [2H]c1c([2H])c([2H])c(-c2nc3nc(n2)n2c4ccccc4c4cccc(c5ccccc5c5cccc6c7ccccc7n3c56)c42)c([2H])c1[2H]] 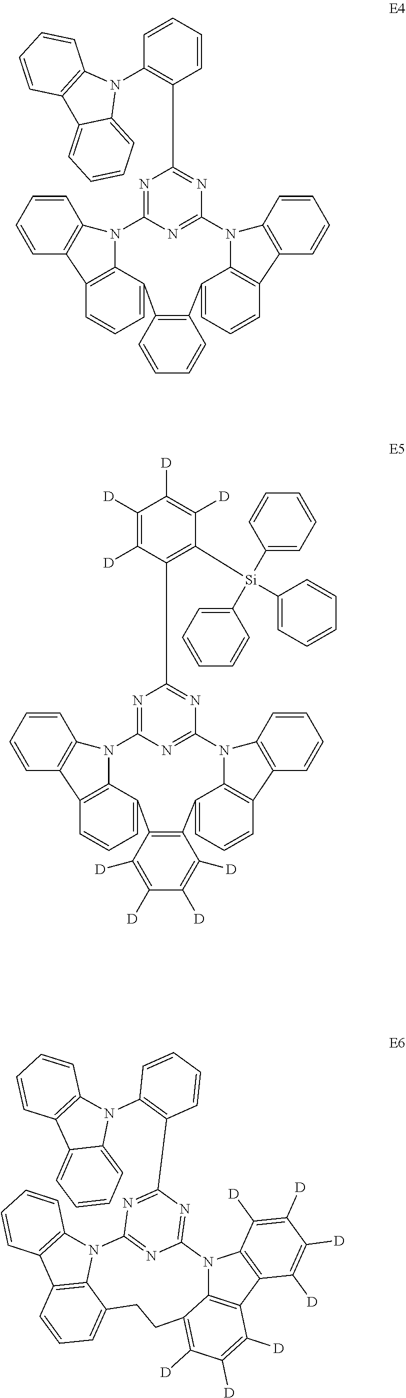 [SMILES: c1ccc(-n2c3ccccc3c3ccccc32)c(-c2nc3nc(n2)n2c4ccccc4c4cccc(c5ccccc5c5cccc6c7ccccc7n3c56)c42)c1 ; [2H]c1c([2H])c([2H])c([Si](c2ccccc2)(c2ccccc2)c2ccccc2)c(-c2nc3nc(n2)n2c4ccccc4c4cccc(c5c([2H])c([2H])c([2H])c([2H])c5c5cccc6c7ccccc7n3c65)c42)c1[2H] ; [2H]c1c([2H])c([2H])c2c(c1[2H])c1c([2H])c([2H])c([2H])c3c1n2-c1nc(-c2ccccc2-n2c4ccccc4c4ccccc42)nc(n1)-n1c2ccccc2c2cccc(c21)CC3] 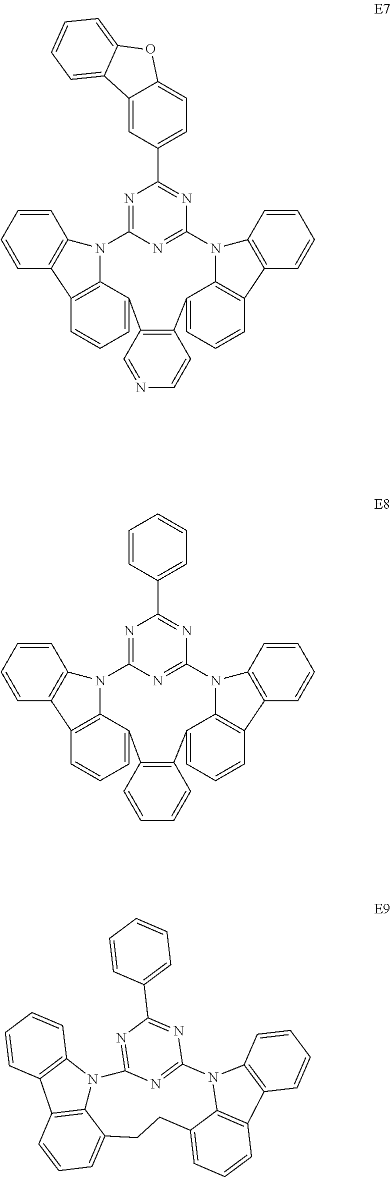 [SMILES: c1ccc2c(c1)oc1ccc(-c3nc4nc(n3)n3c5ccccc5c5cccc(c6cnccc6c6cccc7c8ccccc8n4c67)c53)cc12 ; c1ccc(-c2nc3nc(n2)n2c4ccccc4c4cccc(c5ccccc5c5cccc6c7ccccc7n3c56)c42)cc1 ; c1ccc(-c2nc3nc(n2)-n2c4ccccc4c4cccc(c42)CCc2cccc4c5ccccc5n-3c24)cc1] 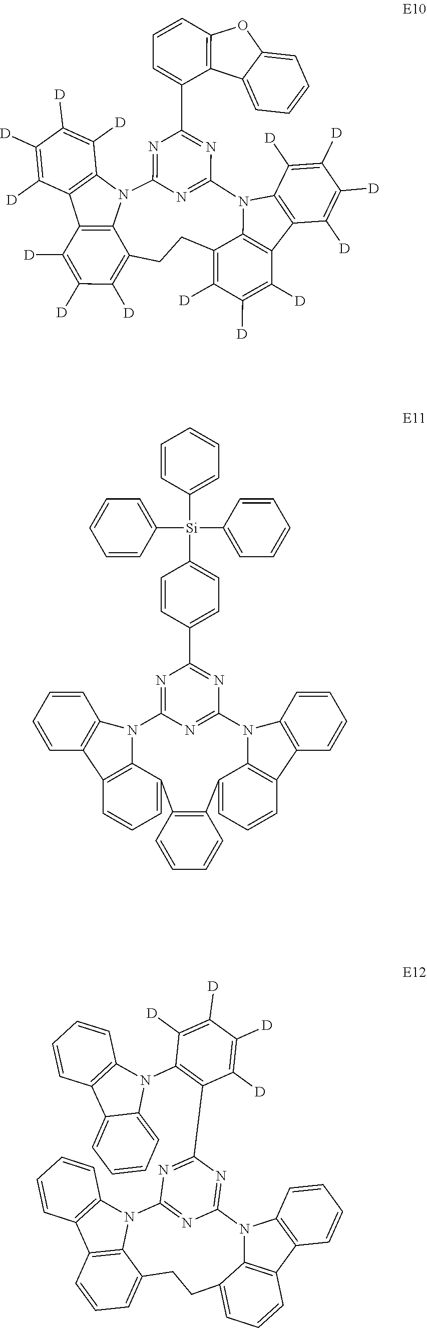 [SMILES: [2H]c1c([2H])c([2H])c2c(c1[2H])c1c([2H])c([2H])c([2H])c3c1n2-c1nc(-c2cccc4oc5ccccc5c24)nc(n1)-n1c2c([2H])c([2H])c([2H])c([2H])c2c2c([2H])c([2H])c([2H])c(c21)CC3 ; c1ccc([Si](c2ccccc2)(c2ccccc2)c2ccc(-c3nc4nc(n3)n3c5ccccc5c5cccc(c6ccccc6c6cccc7c8ccccc8n4c67)c53)cc2)cc1 ; [2H]c1c([2H])c([2H])c(-n2c3ccccc3c3ccccc32)c(-c2nc3nc(n2)-n2c4ccccc4c4cccc(c42)CCc2cccc4c5ccccc5n-3c24)c1[2H]] 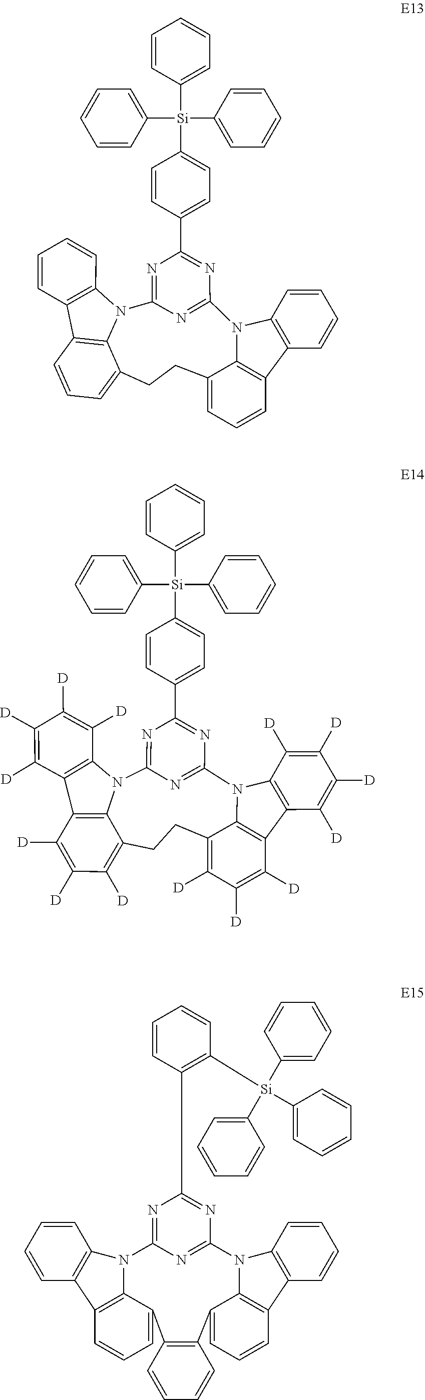 [SMILES: c1ccc([Si](c2ccccc2)(c2ccccc2)c2ccc(-c3nc4nc(n3)-n3c5ccccc5c5cccc(c53)CCc3cccc5c6ccccc6n-4c35)cc2)cc1 ; [2H]c1c([2H])c([2H])c2c(c1[2H])c1c([2H])c([2H])c([2H])c3c1n2-c1nc(-c2ccc([Si](c4ccccc4)(c4ccccc4)c4ccccc4)cc2)nc(n1)-n1c2c([2H])c([2H])c([2H])c([2H])c2c2c([2H])c([2H])c([2H])c(c21)CC3 ; c1ccc([Si](c2ccccc2)(c2ccccc2)c2ccccc2-c2nc3nc(n2)n2c4ccccc4c4cccc(c5ccccc5c5cccc6c7ccccc7n3c56)c42)cc1] 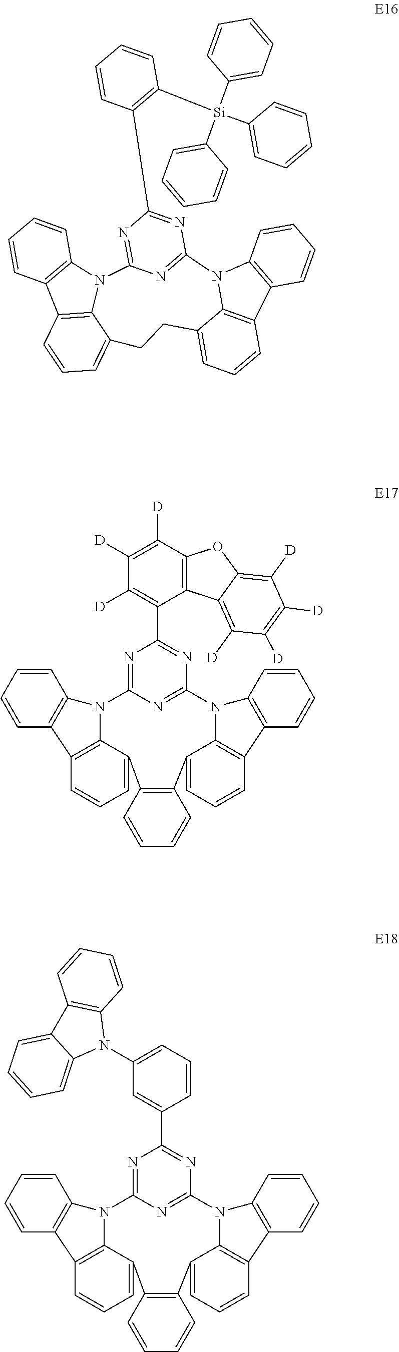 [SMILES: c1ccc([Si](c2ccccc2)(c2ccccc2)c2ccccc2-c2nc3nc(n2)-n2c4ccccc4c4cccc(c42)CCc2cccc4c5ccccc5n-3c24)cc1 ; [2H]c1c([2H])c([2H])c2c(oc3c([2H])c([2H])c([2H])c(-c4nc5nc(n4)n4c6ccccc6c6cccc(c7ccccc7c7cccc8c9ccccc9n5c78)c64)c32)c1[2H] ; c1cc(-c2nc3nc(n2)n2c4ccccc4c4cccc(c5ccccc5c5cccc6c7ccccc7n3c56)c42)cc(-n2c3ccccc3c3ccccc32)c1] 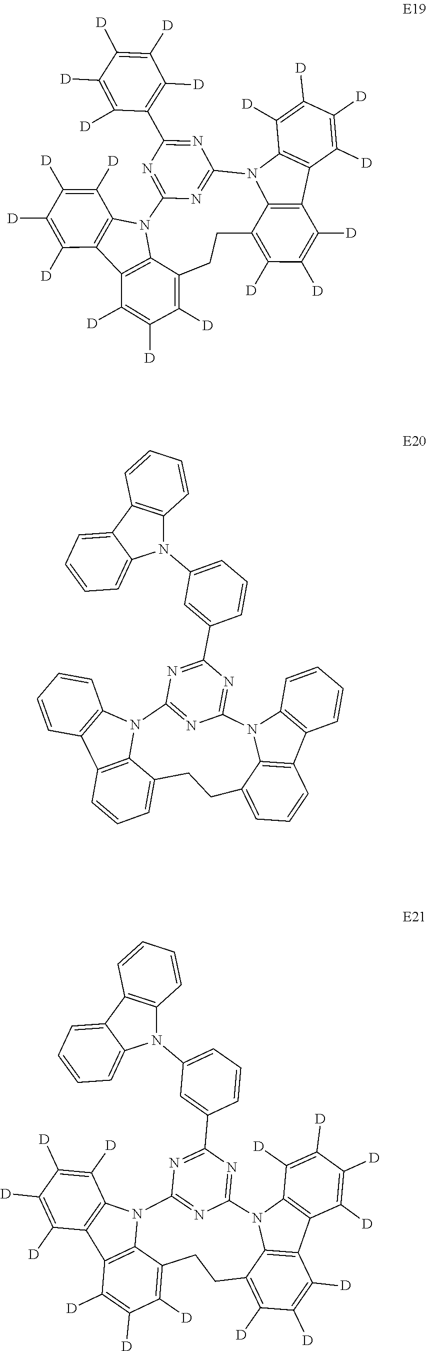 [SMILES: [2H]c1c([2H])c([2H])c(-c2nc3nc(n2)-n2c4c([2H])c([2H])c([2H])c([2H])c4c4c([2H])c([2H])c([2H])c(c42)CCc2c([2H])c([2H])c([2H])c4c5c([2H])c([2H])c([2H])c([2H])c5n-3c24)c([2H])c1[2H] ; c1cc(-c2nc3nc(n2)-n2c4ccccc4c4cccc(c42)CCc2cccc4c5ccccc5n-3c24)cc(-n2c3ccccc3c3ccccc32)c1 ; [2H]c1c([2H])c([2H])c2c(c1[2H])c1c([2H])c([2H])c([2H])c3c1n2-c1nc(-c2cccc(-n4c5ccccc5c5ccccc54)c2)nc(n1)-n1c2c([2H])c([2H])c([2H])c([2H])c2c2c([2H])c([2H])c([2H])c(c21)CC3] 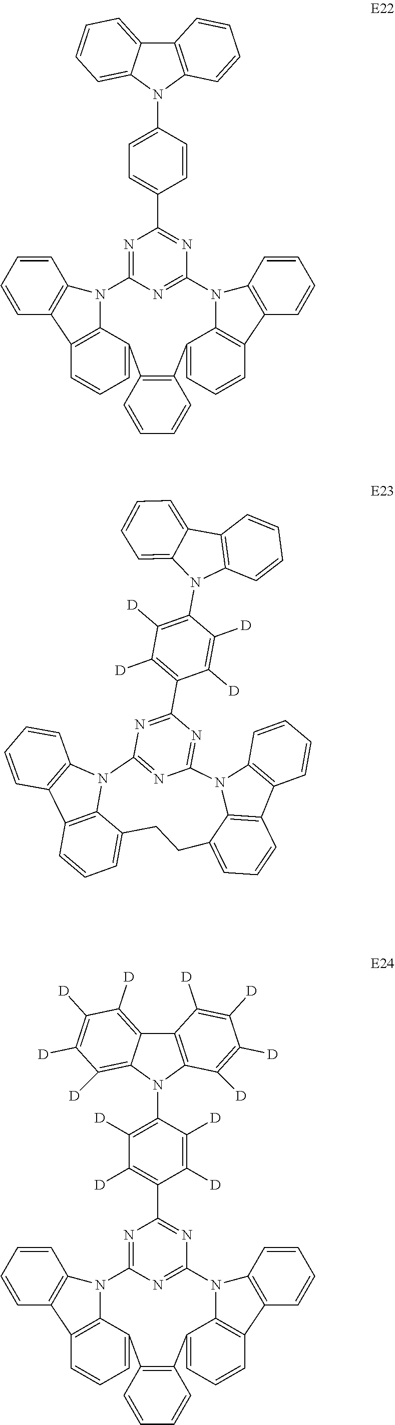 [SMILES: c1ccc2c(c1)c1cccc3c4ccccc4n(c4nc(-c5ccc(-n6c7ccccc7c7ccccc76)cc5)nc(n4)n4c5ccccc5c5cccc2c54)c13 ; [2H]c1c([2H])c(-n2c3ccccc3c3ccccc32)c([2H])c([2H])c1-c1nc2nc(n1)-n1c3ccccc3c3cccc(c31)CCc1cccc3c4ccccc4n-2c13 ; [2H]c1c([2H])c(-n2c3c([2H])c([2H])c([2H])c([2H])c3c3c([2H])c([2H])c([2H])c([2H])c32)c([2H])c([2H])c1-c1nc2nc(n1)n1c3ccccc3c3cccc(c4ccccc4c4cccc5c6ccccc6n2c45)c31] 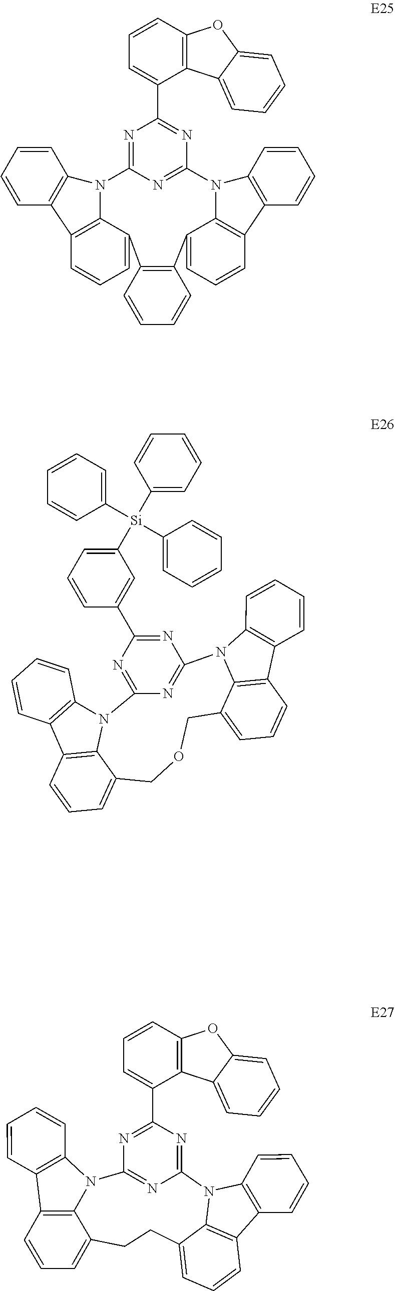 [SMILES: c1ccc2c(c1)oc1cccc(-c3nc4nc(n3)n3c5ccccc5c5cccc(c6ccccc6c6cccc7c8ccccc8n4c67)c53)c12 ; c1ccc([Si](c2ccccc2)(c2ccccc2)c2cccc(-c3nc4nc(n3)-n3c5ccccc5c5cccc(c53)COCc3cccc5c6ccccc6n-4c35)c2)cc1 ; c1ccc2c(c1)oc1cccc(-c3nc4nc(n3)-n3c5ccccc5c5cccc(c53)CCc3cccc5c6ccccc6n-4c35)c12] 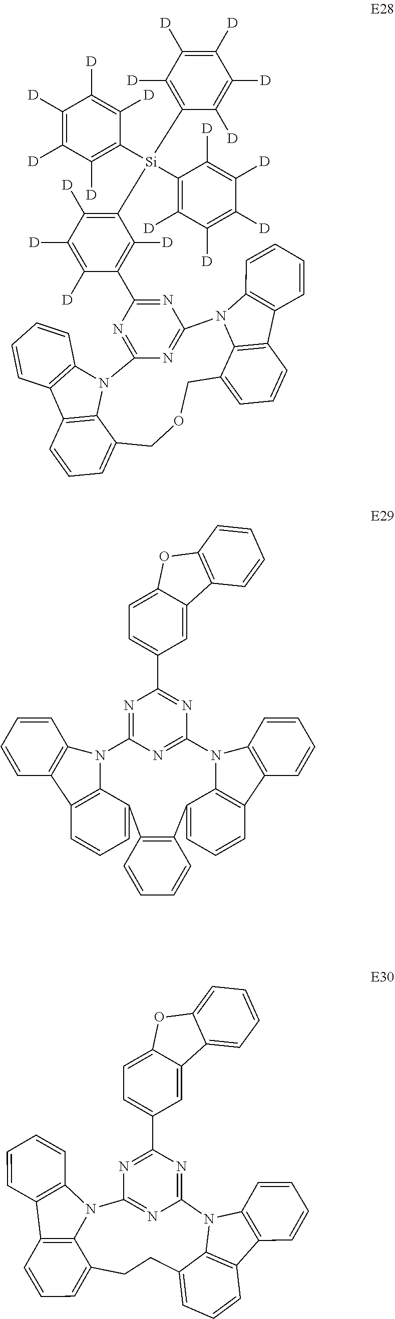 [SMILES: [2H]c1c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c(-c3nc4nc(n3)-n3c5ccccc5c5cccc(c53)COCc3cccc5c6ccccc6n-4c35)c2[2H])c([2H])c1[2H] ; c1ccc2c(c1)oc1ccc(-c3nc4nc(n3)n3c5ccccc5c5cccc(c6ccccc6c6cccc7c8ccccc8n4c67)c53)cc12 ; c1ccc2c(c1)oc1ccc(-c3nc4nc(n3)-n3c5ccccc5c5cccc(c53)CCc3cccc5c6ccccc6n-4c35)cc12] 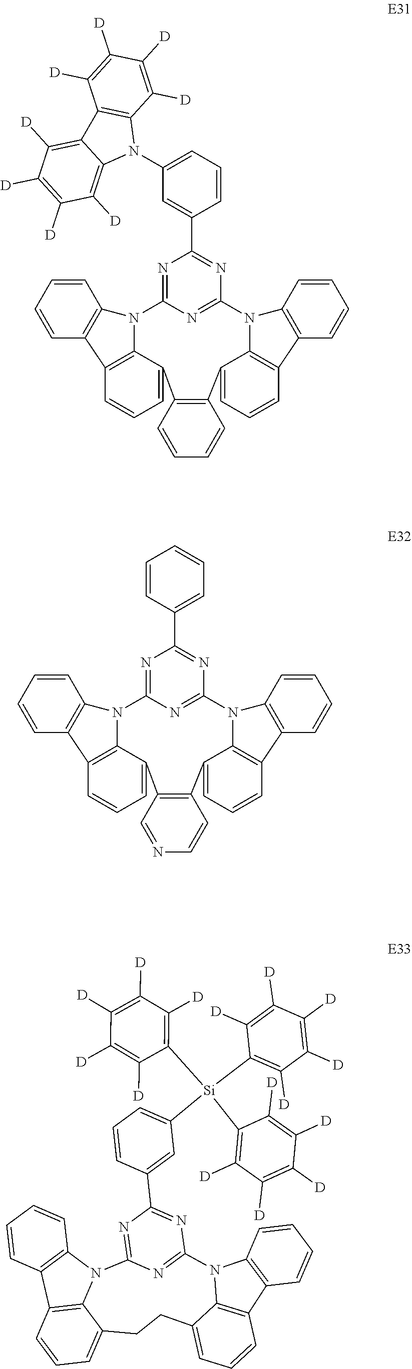 [SMILES: [2H]c1c([2H])c([2H])c2c(c1[2H])c1c([2H])c([2H])c([2H])c([2H])c1n2-c1cccc(-c2nc3nc(n2)n2c4ccccc4c4cccc(c5ccccc5c5cccc6c7ccccc7n3c56)c42)c1 ; c1ccc(-c2nc3nc(n2)n2c4ccccc4c4cccc(c5cnccc5c5cccc6c7ccccc7n3c56)c42)cc1 ; [2H]c1c([2H])c([2H])c([Si](c2cccc(-c3nc4nc(n3)-n3c5ccccc5c5cccc(c53)CCc3cccc5c6ccccc6n-4c35)c2)(c2c([2H])c([2H])c([2H])c([2H])c2[2H])c2c([2H])c([2H])c([2H])c([2H])c2[2H])c([2H])c1[2H]] 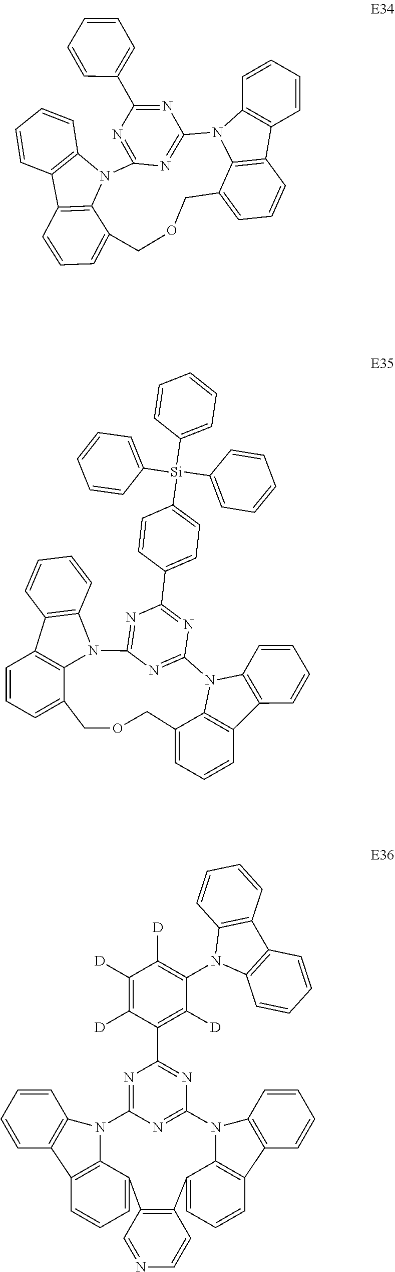 [SMILES: c1ccc(-c2nc3nc(n2)-n2c4ccccc4c4cccc(c42)COCc2cccc4c5ccccc5n-3c24)cc1 ; c1ccc([Si](c2ccccc2)(c2ccccc2)c2ccc(-c3nc4nc(n3)-n3c5ccccc5c5cccc(c53)COCc3cccc5c6ccccc6n-4c35)cc2)cc1 ; [2H]c1c([2H])c(-c2nc3nc(n2)n2c4ccccc4c4cccc(c5cnccc5c5cccc6c7ccccc7n3c56)c42)c([2H])c(-n2c3ccccc3c3ccccc32)c1[2H]] 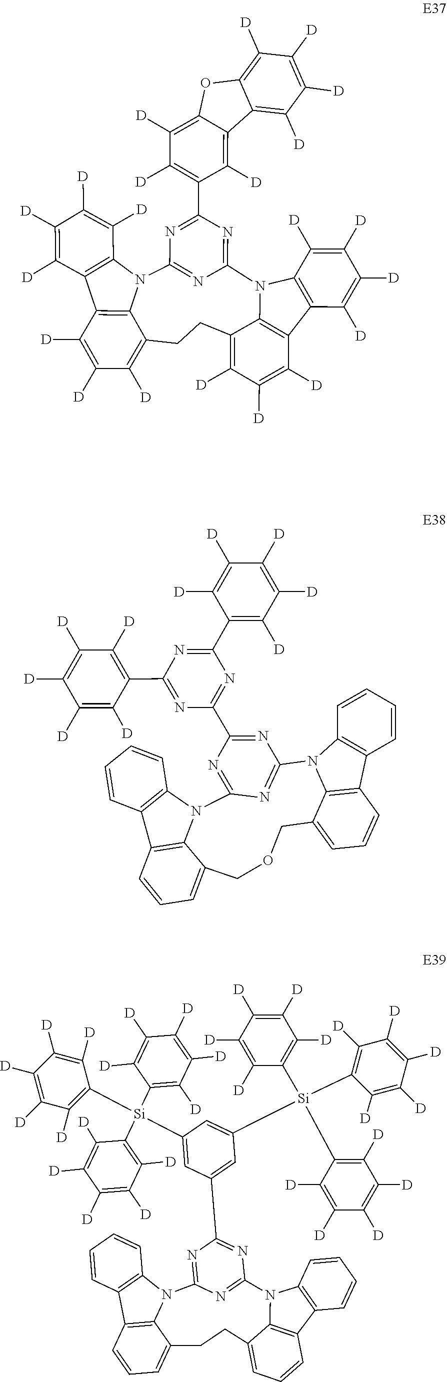 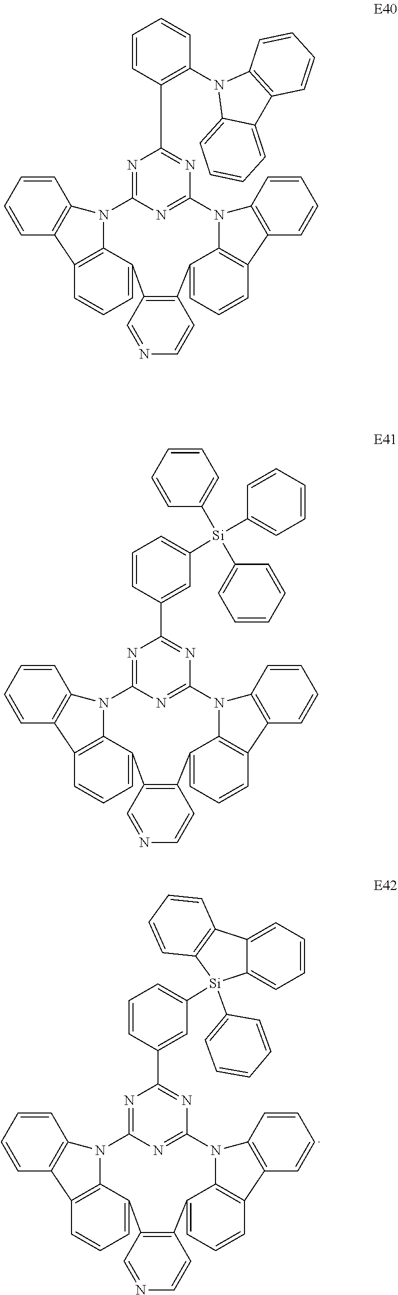 [SMILES: c1ccc(-n2c3ccccc3c3ccccc32)c(-c2nc3nc(n2)n2c4ccccc4c4cccc(c5cnccc5c5cccc6c7ccccc7n3c56)c42)c1 ; c1ccc([Si](c2ccccc2)(c2ccccc2)c2cccc(-c3nc4nc(n3)n3c5ccccc5c5cccc(c6cnccc6c6cccc7c8ccccc8n4c67)c53)c2)cc1 ; c1ccc([Si]2(c3cccc(-c4nc5nc(n4)n4c6ccccc6c6cccc(c7cnccc7c7cccc8c9ccccc9n5c78)c64)c3)c3ccccc3-c3ccccc32)cc1]
